(19) United States
(12) Patent Application Publication
(10) Pub. No.: US 2024/0101605 A1
(43) Pub. Date: Mar. 28, 2024

(54) NEW POLYPEPTIDE

(71) Applicant: AFFIBODY AB, Solna (SE)

(72) Inventors: John LÖFBLOM, Tullinge (SE); Stefan STÅHL, Stockholm (SE); Jonas PERSSON, Stockholm (SE); Olof ERIKSSON, Uppsala (SE); Olov KORSGREN, Uppsala (SE)

(21) Appl. No.: 18/255,215

(22) PCT Filed: Dec. 21, 2021
(86) PCT No.: PCT/EP2021/087036

§ 371 Date: May 31, 2023

(30) Foreign Application Priority Data
Dec. 21, 2020 (EP) 20216069.3
Jul. 30, 2021 (EP) 21188676.7

(51) Int. Cl. C07K 14/00

(52) U.S. Cl. CPC C07K 14/001; C07K 2319/20; C07K 2319/40

(57) ABSTRACT

The present disclosure relates to a class of engineered polypeptides having a binding affinity for CD69 and provides a CD69-binding polypeptide comprising the sequence EX₂X₃X₄AX₆X₇EIX₁₀ X₁₁LPNLX₁₆X₁₇X₁₈QK X₂₁AFKX₂₅X₂₆LKD. The present disclosure also relates to the use of such a CD69-binding polypeptide as a therapeutic, prognostic and/or diagnostic agent.

### FIELD OF THE INVENTION

The present disclosure relates to a class of engineered polypeptides having a binding affinity for CD69. The present disclosure also relates to the use of such a CD69-binding polypeptide as a therapeutic, prognostic and/or diagnostic agent.

### BACKGROUND

The understanding of autoimmune disease, transplant rejection and immunotherapy for malignancies is limited and mainly based on animal models, human tissue biopsies or post-mortem analysis. There are currently no non-invasive methods for the direct study of the immune response in humans, whether to obtain qualitative or quantitative measures of the response.

### Immunology & Type 1 Diabetes

The immune response to an allogeneic cell is well characterized. In contrast, for most other immune-mediated diseases, the underlying mechanisms are only vaguely understood. For example, islet-autoreactive CD8⁺ T cells in peripheral blood were recently found to be equally frequent in patients with recent-onset type 1 diabetes (T1D) and in non-diabetic volunteers (Skowera et al (2015), Diabetes 64(3):916-925).

Many immunologists are currently taking the view that it is of limited value to characterize T cell responses in peripheral blood in autoimmunity, infection and transplantation. Instead, focus is directed to tissue biopsies and, preferentially, on non-invasive, qualitative and quantitative PET imaging of the affected person.

### CD69

One of the earliest cell surface antigens expressed by all T cells following activation is CD69, which is detectable within 1 h after ligation of the T cell receptor/CD3 complex (Radulovic and Niess (2015), J Immunol Res 2015:497056; Kimura et al (2017), Immunol Rev 278:87-100; and Cibrian and Sanchez-Madrid (2017), Eur J Immunol 47:946-953). In addition to being present on mature T cells, CD69 expression is induced upon activation of B cells, natural killer (NK) cells, monocytes, neutrophils and eosinophils.

CD69 is constitutively expressed only by mature thymocytes and platelets, and CD69 is not found on resting circulating leukocytes in humans (Gavioli et al (1992), Cell Immunol 142:186-196).

Previous studies reported that CD69 expression is often detected on cells in samples from inflamed tissues in patients with several different diseases, including T1D, rheumatoid arthritis, psoriasis, asthma, eosinophilic pneumonia, chronic obstructive pulmonary disease (COPD), chronic bronchitis, eosinophilic chronic rhinosinusitis (ECRS), arthritis, sarcoidosis, atopic dermatitis, atherosclerosis, systemic sclerosis, multiple sclerosis, systemic lupus erythematous, granulomatosis with polyangiitis (Wegener's granulomatosis), neuromyelitis optica and autoimmune thyroiditis, as well in transplant biopsies of rejecting grafts and T and NK cells infiltrating tumors (Radulovic and Niess, supra; Kimura et al, supra; and Cibrian and Sanchez-Madrid, supra). These clinical findings indicate that CD69 expression is a valid activation marker for all leukocytes in tissues with ongoing inflammation (both type 1 or type 2 inflammation/active autoimmunity/transplant rejection).

While it is possible to study the autoimmune response using pancreatic biopsies, this invasive approach is associated with a high risk for life threatening complications. Thus, there is a need for agents that can be used for non-invasive studies, for example making use of the presence of CD69 as a marker. There is therefore a need for agents with an affinity for CD69. Also of interest are repeatable and non-invasive methods, in which CD69-binding agents can be used to study the immune response in human pancreas, for example in order to improve our understanding of disease etiology and progression, and to provide novel immune-modulating interventions.

### SUMMARY OF THE INVENTION

It is an object of the present disclosure to provide new CD69-binding agents, which could for example be used for therapeutic, prognostic and diagnostic applications.

It is an object of the present disclosure to provide a new multi-specific agent, such as a bispecific agent, which has affinity for CD69 and at least one additional binding target.

It is an object of the present disclosure to provide a molecule suitable for prognostic and diagnostic applications in relation to autoinflammatory diseases, for example prognostic and diagnostic applications in relation to acute autoimmune diseases, for example prognostic and diagnostic applications in relation to T1D.

In connection with the specific and non-limiting object of providing prognostic and diagnostic applications in relation to T1D, it is noted that the understanding of the autoimmune response in T1D is mainly based on animal models, human tissue biopsies or post-mortem analysis. It is therefore an object of the disclosure to enable repeatable and non-invasive methods for direct monitoring of the immune response in human pancreas, which would improve understanding of T1D etiology and disease progression.

A related object is to enable the efficacy assessment of immune-modulating interventions in autoinflammatory diseases, for example in acute autoimmune diseases, for example in T1D. Another related object is to enable quantitative assessment of inflammatory processes in such conditions.

It is an object of the present disclosure to provide a molecule allowing for therapy of various forms of autoinflammatory diseases, including acute autoimmune diseases, while alleviating the drawbacks of current therapies.

These and other objects, which are evident to the skilled person from the present disclosure, are met by the different aspects of the invention as claimed in the appended claims and as generally disclosed herein.

Thus, in the first aspect of the disclosure, there is provided a CD69-binding polypeptide, comprising a CD69-binding motif BM, which motif consists of an amino acid sequence selected from:

i)
(SEQ ID NO: 168)
EX₂X₃X₄AX₆X₇EIX₁₀ X₁₁LPNLX₁₆
X₁₇X₁₈QK X₂₁AFKX₂₅X₂₆LKD

wherein, independently of each other,
X₂ is selected from F, H, V and W;
X₃ is selected from A, D, E, H, N, Q, S, T, Y;
X₄ is selected from A, D, E, H, K, M, N, S, V, W and Y;
X₆ is selected from M, W and Y;
X₇ is selected from A, H, K, N, Q, R, W and Y;
X₁₀ is selected from L and R;
X₁₁ is selected from A, H, K, R, S and V;
X₁₆ is selected from N and T;
X₁₇ is selected from A, D, K, Q, S and V;
X₁₈ is selected from W and Y;
X₂₁ is selected from E and S;
X₂₅ is selected from H and T; and
X₂₆ is selected from K and S;
   and
   ii) an amino acid sequence which has at least 93% identity to the sequence defined in i).

The above definition of a class of sequence related, CD69-binding polypeptides is based on a statistical analysis of a number of random polypeptide variants of a parent scaffold, that were selected for their interaction with CD69 in selection experiments. The identified CD69-binding motif, or “BM”, corresponds to the target binding region of the parent scaffold, which region constitutes two alpha helices within a three-helical bundle protein domain. In the parent scaffold, the varied amino acid residues of the two BM helices constitute a binding surface for interaction with the constant Fc part of antibodies. In the present disclosure, the random variation of binding surface residues and subsequent selection of variants have replaced the Fc interaction capacity with a capacity for interaction with CD69.

As the skilled person will realize, the function of any polypeptide, such as the CD69-binding capacity of the polypeptide of the present disclosure, is dependent on the tertiary structure of the polypeptide. It is therefore possible to make minor changes to the sequence of amino acids in a polypeptide without affecting the function thereof. Thus, the disclosure encompasses modified variants of the CD69-binding polypeptide, which have retained CD69-binding characteristics.

In this way, encompassed by the present disclosure is a CD69-binding polypeptide comprising an amino acid sequence with 93% or greater identity to a polypeptide as defined in i). In some embodiments, said polypeptide may comprise a sequence which is at least 96% identical to a polypeptide as defined in i). For example, it is possible that an amino acid residue belonging to a certain functional grouping of amino acid residues (e.g. hydrophobic, hydrophilic, polar etc) could be exchanged for another amino acid residue from the same functional group.

In some embodiments, such changes may be made in any position of the sequence of the CD69-binding polypeptide as disclosed herein. In other embodiments, such changes may be made only in the non-variable positions, also denoted scaffold amino acid residues. In such cases, changes are not allowed in the variable positions. In other embodiments, such changes may be only in the variable positions.

According to one definition of such “variable positions”, these are positions denoted with an “X” in sequence i) as defined above.

According to another definition, such “variable positions” are those positions that are randomized in a selection library of Z variants prior to selection, and may thus for example be positions 2, 3, 4, 6, 7, 10, 11, 17, 18, 20, 21, 24, 25 and 28 in sequence i), as illustrated in Example 1 and FIG. 1. This definition of “variable positions” does not include positions 16 and 26, which are scaffold positions in this context, albeit allowed to be either one of two alternatives in each position. Reference is made to Nord et al. (1995), Prot Eng 8:601-608, and Löfblom et al. (2010), FEBS Letters, 584:2670-2680. Like the CD69-binding Z variants of the present disclosure, the polypeptides disclosed in Nord et al. and Löfblom et al. are also based on a scaffold from the Z derivative of domain B of protein A from Staphylococcus aureus, although directed to other targets. As shown in Nord et al. (see for example FIG. 4), the amino acids in positions 23 (corresponding to position 16 in the instant CD69-binding motif) and 33 (corresponding to position 26 in the instant CD69-binding motif) are N and S, respectively. As also shown in Löfblom et al., polypeptides with amino acid residues N and S in positions 23 and 33, respectively (corresponding to positions 16 and 26 in the instant CD69-binding motif; see FIG. 2 of Löfblom et al), and polypeptides with amino acid residues T and K in positions 23 and 33, respectively, all have a maintained basic structure and function. Thus, in the context of this definition of “variable positions”, the amino acid residues at positions 16 and 26 form part of the common scaffold, and it is contemplated to have either N or T in scaffold position 16 and either S or K in scaffold position 26. Neither the presence of N and S, nor that of T and K, in the respective position negatively effects the structure and function of the polypeptide.

The term “% identity”, as used throughout the specification, may for example be calculated as follows. The query sequence is aligned to the target sequence using the CLUSTAL W algorithm (Thompson et al, (1994) Nucleic Acids Research, 22: 4673-4680). A comparison is made over the window corresponding to the shortest of the aligned sequences. In one embodiment, the target sequence is the shortest of the aligned sequences. In an alternative embodiment, the query sequence is the shortest of the aligned sequences. The amino acid residues at each position are compared and the percentage of positions in the query sequence that have identical correspondences in the target sequence is reported as % identity.

In another embodiment, there is provided a CD69-binding polypeptide wherein, in sequence i),
X₂ is F;
X₃ is selected from Q, S and Y;
X₄ is selected from H, M, N and W;
X₆ is selected from M and W;
X₇ is selected from K, Q and W;
X₁₀ is selected from L and R;
X₁₁ is selected from A, H, K and V;
X₁₆ is selected from N and T;
X₁₇ is selected from A, K, Q and S;
X₁₈ is selected from W and Y;
X₂₁ is E;
X₂₅ is T; and
X₂₆ is selected from K and S.

As used herein, “Xₙ” and “Xₘ” are used to indicate amino acids in positions n and m in the sequence i) as defined above, wherein n and m are integers which indicate the position of an amino acid within said sequence as counted from the N-terminal end of said sequence. For example, X₂ and X₆ indicate the amino acid in position two and six, respectively, from the N-terminal end of sequence i).

In embodiments according to the first aspect, there are provided polypeptides wherein Xₙ in sequence i) is independently selected from a group of possible residues according to Table 1. The skilled person will appreciate that Xₙ may be selected from any one of the listed groups of possible residues and that this selection is independent from the selection of amino acids in Xₘ, wherein n≠m. Thus, any of the listed possible residues in position Xₙ in Table 1 may be independently combined with any of the listed possible residues any other variable position in Table 1.

The skilled person will appreciate that Table 1 is to be read as follows: In one embodiment according to the first aspect, there is provided a polypeptide wherein amino acid residue “Xₙ” in sequence i) is selected from “Possible residues”. Thus, Table 1 discloses several specific and individualized embodiments of the first aspect of the present disclosure. For example, in one embodiment according to the first aspect, there is provided a polypeptide wherein X₂ in sequence i) is selected from F, H, V and W, and in another embodiment according to the first aspect, there is provided a polypeptide wherein X₂ in sequence i) is selected from F and W. For avoidance of doubt, the listed embodiments may be freely combined in yet other embodiments. For example, one such combined embodiment is a polypeptide in which X₂ is selected from F and W, while X₄ is selected from H, M, N and W.

TABLE 1

| Xₙ | Possible residues |
|---|---|
| X₂ | F, H, V and W |
| X₂ | F, H and W |
| X₂ | F, V and W |
| X₂ | F and W |
| X₂ | F |
| X₃ | A, D, E, H, N, Q, S, T, Y |
| X₃ | A, D, E, H, Q, S, T, Y |
| X₃ | A, D, H, N, Q, S, T, Y |
| X₃ | A, E, H, N, Q, S, T, Y |
| X₃ | A, H, N, Q, S, T, Y |
| X₃ | A, H, Q, S, T, Y |
| X₃ | A, H, Q, S and Y |
| X₃ | Q, S and Y |
| X₃ | H and Y |
| X₃ | Y |
| X₄ | A, D, E, H, K, M, N, S, V, W and Y |
| X₄ | A, D, E, H, M, N, S, V, W and Y |
| X₄ | A, D, E, H, K, M, N, S, V and W |
| X₄ | A, D, E, H, M, N, S, V and W |
| X₄ | A, D, E, H, M, N, V and W |
| X₄ | D, E, H, M, N, S, V and W |
| X₄ | D, E, H, M, N, S and W |
| X₄ | A, D, H, M, N and W |
| X₄ | H, M, N and W |
| X₄ | A, D and H |
| X₄ | H |
| X₄ | M |
| X₄ | N |
| X₄ | W |
| X₆ | M, W and Y |
| X₆ | M and W |
| X₆ | M |
| X₆ | W |
| X₇ | A, H, K, N, Q, R, W and Y |
| X₇ | A, H, K, N, Q, R and W |
| X₇ | A, K, N, Q, R, W and Y |
| X₇ | A, K, N, Q, R and W |
| X₇ | A, K, Q, R and W |
| X₇ | A, K, Q and W |
| X₇ | K, N and Q |
| X₇ | K, Q and W |
| X₇ | K and R |
| X₇ | K and N |
| X₇ | K |
| X₇ | N |
| X₇ | Q |
| X₇ | W |
| X₁₀ | L and R |
| X₁₀ | L |
| X₁₀ | R |
| X₁₁ | A, H, K, R, S and V |
| X₁₁ | A, H, K, R and V |
| X₁₁ | A, H, K, S and V |
| X₁₁ | A, H, K and V |
| X₁₁ | H, K, R and V |
| X₁₁ | K and R |
| X₁₁ | K |
| X₁₁ | A |
| X₁₁ | H |
| X₁₁ | V |
| X₁₁ | R |
| X₁₆ | N and T |
| X₁₆ | N |
| X₁₆ | T |
| X₁₇ | A, D, K, Q, S and V |
| X₁₇ | A, K, Q, S and V |
| X₁₇ | A, D, K, Q and S |
| X₁₇ | A, K, Q and S |
| X₁₇ | A, K and Q |
| X₁₇ | K and Q |
| X₁₇ | K |
| X₁₇ | A |
| X₁₇ | Q |
| X₁₇ | S |
| X₁₈ | W and Y |
| X₁₈ | W |
| X₁₈ | Y |
| X₂₁ | E and S |
| X₂₁ | E |
| X₂₅ | H and T |
| X₂₅ | T |
| X₂₆ | K and S |
| X₂₆ | K |
| X₂₆ | S |

In one particular embodiment according to the first aspect, there is provided a polypeptide wherein sequence i) fulfills at least five of the ten conditions I-X:
I. X₂ is F;
II. X₃ is Y;
III. X₄ is H, Nor W;
IV. X₆ is M or W;
V. X₁₀ is L;
VI. X₁₁ is K;
VII X₁₇ is K or Q;
VIII. X₁₈ is Y;
IX. X₂₁ is E; and
X. X₂₅ is T.

In one embodiment, sequence i) fulfills at least six of the ten conditions I-X, such as at least seven of the ten conditions I-X, such as least eight of the ten conditions I-X, such as least nine of the ten conditions I-X. In one particular embodiment, sequence i) fulfills all of the ten conditions I-X.

In some embodiments of a CD69-binding polypeptide according to the first aspect, X₃ is Y, X₄ is H and X₁₁ is K. In some embodiments, X₃ is Y, X₄ is H and X₂₁ is E. In some embodiments, X₃ is Y, X₁₁ is K and X₁₈ is Y. In some embodiments, X₁₁ is K, X₁₈ is Y and X₂₁ is E.

As described in detail in the experimental section to follow, the selection of CD69-binding polypeptide variants led the inventors to identify a number of individual CD69-binding motif (BM) sequences. These amino acid sequences constitute individual embodiments of sequence i) according to this aspect. The sequences of individual CD69-binding motifs correspond to amino acid positions 8-36 in SEQ ID NO:1-144 presented in FIG. 1. With reference to FIG. 1, it is to be noted that the binding motif sequences are pair-wise identical in SEQ ID NO:1 and 73, SEQ ID NO:2 and 74, and so on to SEQ ID NO:72 and 144. In other words, any reference to the binding motif sequence from position 8 to position 36 in any one of SEQ ID NO:1-72 also encompasses the identical sequences in any one of SEQ ID NO:73-144, respectively. Hence, in one embodiment of the CD69-binding polypeptide according to this aspect, sequence i) corresponds to the sequence from position 8 to position 36 in a sequence selected from the group consisting of SEQ ID NO:1-72. In one embodiment of the CD69-binding polypeptide according to this aspect, sequence i) corresponds to the sequence from position 8 to position 36 in a sequence selected from the group consisting of SEQ ID NO:1-29. In a more specific embodiment of the CD69-binding polypeptide according to this aspect, sequence i) corresponds to the sequence from position 8 to position 36 in a sequence selected from the group consisting of SEQ ID NO:1-28. In a yet more specific embodiment of the CD69-binding polypeptide according to this aspect, sequence i) corresponds to the sequence from position 8 to position 36 in a sequence selected from the group consisting of SEQ ID NO:1-26. In another more specific embodiment of the CD69-binding polypeptide according to this aspect, sequence i) corresponds to the sequence from position 8 to position 36 in a sequence selected from the group consisting of SEQ ID NO:1-6, for example selected from the group consisting of SEQ ID NO:1-2 and 6, for example selected from the group consisting of SEQ ID NO:1-2, for example being SEQ ID NO:1.

In some embodiments of the present disclosure, the BM as defined above “forms part of” a three-helix bundle protein domain. This is understood to mean that the sequence of the BM is “inserted” into or “grafted” onto the sequence of the original three-helix bundle domain, such that the BM replaces a similar structural motif in the original domain. For example, without wishing to be bound by theory, the BM is thought to constitute two of the three helices of a three-helix bundle and can therefore replace such a two-helix motif within any three-helix bundle. As the skilled person will realize, the replacement of two helices of the three-helix bundle domain by the two BM helices has to be performed so as not to affect the basic structure of the polypeptide. That is, the overall folding of the Ca backbone of the polypeptide according to this embodiment of the invention is substantially the same as that of the three-helix bundle protein domain of which it forms a part, e.g. having the same elements of secondary structure in the same order etc. Thus, a BM according to the present disclosure “forms part” of a three-helix bundle domain if the polypeptide according to this embodiment has the same fold as the original domain, implying that the basic structural properties are shared, those properties e.g. resulting in similar CD spectra. The skilled person is aware of other parameters that are relevant.

In particular embodiments, the CD69-binding motif (BM) thus forms part of a three-helix bundle protein domain. For example, the BM may essentially constitute two alpha helices with an interconnecting loop, within said three-helix bundle protein domain. In particular embodiments, said three-helix bundle protein domain is selected from domains of bacterial receptor proteins. Non-limiting examples of such domains are the five different three-helical domains of Protein A from Staphylococcus aureus, such as domain B, and derivatives thereof. In some embodiments, the three-helical bundle protein domain is a variant of protein Z, which is derived from domain B of staphylococcal Protein A (Wahlberg E et al, 2003, PNAS 100(6):3185-3190).

In some embodiments where the CD69-binding polypeptide as disclosed herein forms part of a three-helix bundle protein domain, the CD69-binding polypeptide may comprise a binding module (BMod), the amino acid sequence of which is selected from:

iii) K-[BM]-DPSQSXₐX_{b}LLX_{c} EAKX_{d}LXₑX_{f}X_{g}Q;

wherein
[BM] is a CD69-binding motif as defined herein;
Xₐ is selected from A and S;
X_{b} is selected from E and N;
X_{c} is selected from A, S and C;
X_{d} is selected from K and Q;
Xₑ is selected from E, N and S;
X_{f} is selected from D, E and S; and
X_{g} is selected from A and S; and
   iv) an amino acid sequence which has at least 93% identity to a sequence defined in iii).

In some embodiments, said polypeptide may beneficially exhibit a high structural stability, such as resistance to chemical modifications, to changes in physical conditions and to proteolysis, during production and storage, as well as in vivo.

As discussed above, polypeptides comprising minor changes as compared to the above amino acid sequences, which do not largely affect the tertiary structure and the function of the polypeptide, are also within the scope of the present disclosure. Thus, in some embodiments, sequence iv) has at least 93%, such as at least 95%, such as at least 97% identity to a sequence defined by iii).

In one embodiment, Xₐ in sequence iii) is A.

In one embodiment, Xₐ in sequence iii) is S.

In one embodiment, X_{b} in sequence iii) is N.

In one embodiment, X_{b} in sequence iii) is E.

In one embodiment, X_{c} in sequence iii) is A.

In one embodiment, X_{c} in sequence iii) is S.

In one embodiment, X_{c} in sequence iii) is C.

In one embodiment, X_{d} in sequence iii) is K.

In one embodiment, X_{d} in sequence iii) is Q.

In one embodiment, Xₑ in sequence iii) is E.

In one embodiment, Xₑ in sequence iii) is N.

In one embodiment, Xₑ in sequence iii) is S.

In one embodiment, X_{f} in sequence iii) is D.

In one embodiment, X_{f} in sequence iii) is E.

In one embodiment, X_{f} in sequence iii) is S.

In one embodiment, XₑX_{f} in sequence iii) is selected from EE, ES, SD, SE and SS.

In one embodiment, XₑX_{f} in sequence iii) is ES.

In one embodiment, XₑX_{f} in sequence iii) is SE.

In one embodiment, XₑX_{f} in sequence iii) is SD.

In one embodiment, X_{g} in sequence iii) is A.

In one embodiment, X_{g} in sequence iii) is S.

In one embodiment, in sequence iii), Xₐ is A; X_{b} is N; X_{c} is A and X_{g} is A.

In one embodiment, in sequence iii), Xₐ is S; X_{b} is E; X_{c} is A and X_{g} is A.

In one embodiment, in sequence iii), Xₐ is A; X_{b} is N; X_{c} is C and X_{g} is A.

In one embodiment, in sequence iii), Xₐ is S; X_{b} is E; X_{c} is S and X_{g} is S.

In one embodiment, in sequence iii), Xₐ is S; X_{b} is E; X_{c} is C and X_{g} is S.

In one embodiment, in sequence iii), Xₐ is A; X_{b} is N; X_{c} is A; XₑX_{f} is ND and X_{g} is A.

In one embodiment, in sequence iii), Xₐ is S; X_{b} is E; X_{c} is A; XₑX_{f} is ND and X_{g} is A.

In one embodiment, in sequence iii), Xₐ is A; X_{b} is N; X_{c} is C; XₑX_{f} is ND and X_{g} is A.

In one embodiment, in sequence iii), Xₐ is S; X_{b} is E; X_{c} is S; XₑX_{f} is ND and X_{g} is S.

In one embodiment, in sequence iii), Xₐ is S; X_{b} is E; X_{c} is C; XₑX_{f} is ND and X_{g} is S.

In one embodiment, in sequence iii), Xₐ is A; X_{b} is N; X_{c} is A; XₑX_{f} is SE and X_{g} is A.

In one embodiment, in sequence iii), Xₐ is S; X_{b} is E; X_{c} is A; XₑX_{f} is SE and X_{g} is A.

In one embodiment, in sequence iii), Xₐ is A; X_{b} is N; X_{c} is C; XₑX_{f} is SE and X_{g} is A.

In one embodiment, in sequence iii), Xₐ is S; X_{b} is E; X_{c} is S; XₑX_{f} is SE and X_{g} is S.

In one embodiment, in sequence iii), Xₐ is S; X_{b} is E; X_{c} is C; XₑX_{f} is SE and X_{g} is S.

In one embodiment, in sequence iii), Xₐ is A; X_{b} is N; X_{c} is A; XₑX_{f} is SD and X_{g} is A.

In one embodiment, in sequence iii), Xₐ is S; X_{b} is E; X_{c} is A; XₑX_{f} is SD and X_{g} is A.

In one embodiment, in sequence iii), Xₐ is A; X_{b} is N; X_{c} is C; XₑX_{f} is SD and X_{g} is A.

In one embodiment, in sequence iii), Xₐ is S; X_{b} is E; X_{c} is S; XₑX_{f} is SD and X_{g} is S.

In one embodiment, in sequence iii), Xₐ is S; X_{b} is E; X_{c} is C; XₑX_{f} is SD and X_{g} is S.

In yet a further embodiment, sequence iii) corresponds to the sequence from position 7 to position 55 in a sequence selected from the group consisting of SEQ ID NO:1-72 presented in FIG. 1. Hence, in one embodiment of the CD69-binding polypeptide according to this aspect, sequence iii) corresponds to the sequence from position 7 to position 55 in a sequence selected from the group consisting of SEQ ID NO:1-72. In one embodiment of the CD69-binding polypeptide according to this aspect, sequence iii) corresponds to the sequence from position 7 to position 55 in a sequence selected from the group consisting of SEQ ID NO:1-29. In a more specific embodiment of the CD69-binding polypeptide according to this aspect, sequence iii) corresponds to the sequence from position 7 to position 55 in a sequence selected from the group consisting of SEQ ID NO:1-28. In a yet more specific embodiment of the CD69-binding polypeptide according to this aspect, sequence iii) corresponds to the sequence from position 7 to position 55 in a sequence selected from the group consisting of SEQ ID NO:1-26. In another more specific embodiment of the CD69-binding polypeptide according to this aspect, sequence iii) corresponds to the sequence from position 7 to position 55 in a sequence selected from the group consisting of SEQ ID NO:1-6, for example selected from the group consisting of SEQ ID NO:1-2 and 6, for example selected from the group consisting of SEQ ID NO:1-2, for example being SEQ ID NO:1.

In a further, alternative, embodiment, sequence iii) corresponds to the sequence from position 7 to position 55 in a sequence selected from the group consisting of SEQ ID NO:73-144 presented in FIG. 1. Hence, in one embodiment of the CD69 binding polypeptide according to this aspect, sequence iii) corresponds to the sequence from position 7 to position 55 in a sequence selected from the group consisting of SEQ ID NO:73-144. In one embodiment of the CD69-binding polypeptide, sequence iii) corresponds to the sequence from position 7 to position 55 in a sequence selected from the group consisting of SEQ ID NO:73-101. In a more specific embodiment of the CD69-binding polypeptide according to this aspect, sequence iii) corresponds to the sequence from position 7 to position 55 in a sequence selected from the group consisting of SEQ ID NO: 73-100. In a yet more specific embodiment of the CD69-binding polypeptide according to this aspect, sequence iii) corresponds to the sequence from position 7 to position 55 in a sequence selected from the group consisting of SEQ ID NO:73-98. In another more specific embodiment of the CD69-binding polypeptide according to this aspect, sequence iii) corresponds to the sequence from position 7 to position 55 in a sequence selected from the group consisting of SEQ ID NO:73-78, for example selected from the group consisting of SEQ ID NO:73-74 and 78, for example selected from the group consisting of SEQ ID NO:73-74, for example being SEQ ID NO:73.

Also, in a further embodiment, there is provided a CD69-binding polypeptide, which comprises an amino acid sequence selected from:

v)
FN-[BMod]-AP;

wherein [BMod] is a CD69-binding module as defined herein; and
vi) an amino acid sequence which has at least 90% identity to a sequence defined in v).

Alternatively, there is provided a CD69-binding polypeptide, which comprises an amino acid sequence selected from:

vii)
YA-[BMod]-AP;

wherein [BMod] is a CD69-binding module as defined herein; and
viii) an amino acid sequence which has at least 90% identity to a sequence defined in vii).

For example, in one embodiment there is provided a CD69-binding polypeptide selected from the group consisting of

ix)
FNK-[BM]-DPSQS ANLLA EAKX_{d} L NDAQA P;

wherein [BM] is a CD69-binding motif as defined above and X_{d} is selected from K and Q; and
x) an amino acid sequence which has at least 90% identity to a sequence defined in ix).

In another embodiment, there is provided a CD69-binding polypeptide selected from the group consisting of

xi)
FNK-[BM]-DPSQS SELLS EAKX_{d}L NDSQA P;

wherein [BM] is a CD69-binding motif as defined above and X_{d} is selected from K and Q; and
xii) an amino acid sequence which has at least 90% identity to a sequence defined in xi).

In another embodiment, there is provided a CD69-binding polypeptide selected from the group consisting of

xiii)
FAK-[BM]-DPSQS SELLX_{c} EAKKL SESQA P;

wherein [BM] is a CD69-binding motif as defined above and X_{c} is selected from A, S and C;
xiv) an amino acid sequence which has at least 90% identity to a sequence defined in xiii).

In yet another embodiment, there is provided a CD69-binding polypeptide selected from the group consisting of

xv)
YAK-[BM]-DPSQS SELLX_{c} EAKKL NDSQA P;

wherein [BM] is a CD69-binding motif as defined above and X_{c} is selected from A, S and C;
xvi) and an amino acid sequence which has at least 90% identity to a sequence defined in xv).

As discussed above, polypeptides comprising minor changes as compared to the above amino acid sequences, which do not largely affect the tertiary structure and the function of the polypeptide, also fall within the scope of the present disclosure. Thus, in some embodiments, sequence vi), viii), x), xii), xiv) or xvi) may for example be at least 90%, such as at least 92%, such as at least 94%, such as at least 96%, such as at least 98% identical to a sequence defined by v, vii), ix), xi), xiii) and xv), respectively.

In some embodiments, the CD69-binding motif may form part of a polypeptide comprising an amino acid sequence selected from

ADNNFNK-[BM]-DPSQSANLLSEAKKLNESQAPK;
ADNKFNK-[BM]-DPSQSANLLAEAKKLNDAQAPK;
ADNKFNK-[BM]-DPSVSKEILAEAKKLNDAQAPK;
ADAQQNNFNK-[BM]-DPSQSTNVLGEAKKLNESQAPK;
AQHDE-[BM]-DPSQSANVLGEAQKLNDSQAPK;
VDNKFNK-[BM]-DPSQSANLLAEAKKLNDAQAPK
VDNKFNK-[BM]-DPSQSSELLSEAKQLNDSQAPK;
VDNKFNK-[BM]-DPSQSSELLSEAKKLNDSQAPK
AEAKYAK-[BM]-DPSESSELLSEAKKLNKSQAPK;
VDAKYAK-[BM]-DPSQSSELLAEAKKLNDAQAPK;
VDAKYAK-[BM]-DPSQSSELLAEAKKLNDSQAPK;
AEAKYAK-[BM]-DPSQSSELLSEAKKLNDSQAPK;
AEAKYAK-[BM]-DPSQSSELLSEAKKLNDSQAP;
AEAKFAK-[BM]-DPSQSSELLSEAKKLNDSQAPK;
AEAKFAK-[BM]-DPSQSSELLSEAKKLNDSQAP;
AEAKYAK-[BM]-DPSQSSELLAEAKKLNDAQAPK;
AEAKYAK-[BM]-DPSQSSELLSEAKKLSESQAPK;
AEAKYAK-[BM]-DPSQSSELLSEAKKLSESQAP;
AEAKFAK-[BM]-DPSQSSELLSEAKKLSESQAPK;
AEAKFAK-[BM]-DPSQSSELLSEAKKLSESQAP;
AEAKYAK-[BM]-DPSQSSELLAEAKKLSEAQAPK;
AEAKYAK-[BM]-QPEQSSELLSEAKKLSESQAPK;
AEAKYAK-[BM]-DPSQSSELLSEAKKLESSQAPK;
AEAKYAK-[BM]-DPSQSSELLSEAKKLESSQAP;
AEAKYAK-[BM]-DPSQSSELLAEAKKLESAQAPK;
AEAKYAK-[BM]-QPEQSSELLSEAKKLESSQAPK;
AEAKYAK-[BM]-DPSQSSELLSEAKKLSDSQAPK;
AEAKYAK-[BM]-DPSQSSELLSEAKKLSDSQAP;
AEAKYAK-[BM]-DPSQSSELLAEAKKLSDSQAPK;
AEAKYAK-[BM]-DPSQSSELLAEAKKLSDAQAPK;
AEAKYAK-[BM]-QPEQSSELLSEAKKLSDSQAPK;
VDAKYAK-[BM]-DPSQSSELLSEAKKLNDSQAPK;
VDAKYAK-[BM]-DPSQSSELLAEAKKLNDAQAPK;
VDAKYAK-[BM]-DPSQSSELLSEAKKLSESQAPK;
VDAKYAK-[BM]-DPSQSSELLAEAKKLSEAQAPK;
VDAKYAK-[BM]-QPEQSSELLSEAKKLSESQAPK;
VDAKYAK-[BM]-DPSQSSELLSEAKKLESSQAPK;
VDAKYAK-[BM]-DPSQSSELLAEAKKLESAQAPK;
VDAKYAK-[BM]-QPEQSSELLSEAKKLESSQAPK;
VDAKYAK-[BM]-DPSQSSELLSEAKKLSDSQAPK;
VDAKYAK-[BM]-DPSQSSELLAEAKKLSDSQAPK;
VDAKYAK-[BM]-DPSQSSELLAEAKKLSDAQAPK;
VDAKYAK-[BM]-QPEQSSELLSEAKKLSDSQAPK;
VDAKYAK-[BM]-DPSQSSELLAEAKKLNKAQAPK;
AEAKYAK-[BM]-DPSQSSELLAEAKKLNKAQAPK;
and
ADAKYAK-[BM]-DPSQSSELLSEAKKLNDSQAPK;

wherein [BM] is a CD69-binding motif as defined herein.

In one embodiment, the CD69-binding polypeptide comprises an amino acid sequence selected from:

xvii)
VDNKFNK-[BM]-DPSQSSELLSEAKQLNDSQAPK;

wherein [BM] is a CD69-binding motif as defined herein; and
xviii) an amino acid sequence which has at least 89% identity to the sequence defined in xvii).

In one embodiment, sequence xvii) in such a polypeptide may for example be selected from the group consisting of SEQ ID NO:73-144 presented in FIG. 1. In one embodiment of the CD69-binding polypeptide, sequence xvii) is selected from the group consisting of SEQ ID NO:73-101. In a more specific embodiment of the CD69-binding polypeptide according to this aspect, sequence xvii) is selected from the group consisting of SEQ ID NO: 73-100. In a yet more specific embodiment of the CD69-binding polypeptide according to this aspect, sequence xvii) is selected from the group consisting of SEQ ID NO:73-98. In another more specific embodiment of the CD69-binding polypeptide according to this aspect, sequence xvii) is selected from the group consisting of SEQ ID NO:73-78, for example selected from the group consisting of SEQ ID NO:73-74 and 78, for example selected from the group consisting of SEQ ID NO:73-74, for example being SEQ ID NO:73.

In one embodiment, the CD69-binding polypeptide comprises an amino acid sequence selected from:

xix)
VDNKFNK-[BM]-DPSQSANLLAEAKKLNDAQAPK;

wherein [BM] is a CD69-binding motif as defined herein; and
xx) an amino acid sequence which has at least 89% identity to the sequence defined in xix).

In one embodiment, sequence xix) in such a polypeptide may for example be selected from the group consisting of SEQ ID NO:1-72 presented in FIG. 1. In one embodiment of the CD69-binding polypeptide, sequence xix) is selected from the group consisting of SEQ ID NO:1-29. In a more specific embodiment of the CD69-binding polypeptide according to this aspect, sequence xix) is selected from the group consisting of SEQ ID NO: 1-28. In a yet more specific embodiment of the CD69-binding polypeptide according to this aspect, sequence xix) is selected from the group consisting of SEQ ID NO:1-26. In another more specific embodiment of the CD69-binding polypeptide according to this aspect, sequence xix) is selected from the group consisting of SEQ ID NO:1-6, for example selected from the group consisting of SEQ ID NO:1-2 and 6, for example selected from the group consisting of SEQ ID NO:1-2, for example being SEQ ID NO:1.

In one embodiment, the CD69-binding polypeptide comprises an amino acid sequence selected from:

xxi)
VDNKFNK-[BM]-DPSQSSELLSEAKKLNDSQAPK;

wherein [BM] is a CD69-binding motif as defined herein; and
xxii) an amino acid sequence which has at least 89% identity to the sequence defined in xxi).

In one embodiment, the CD69-binding polypeptide comprises an amino acid sequence selected from:

xxiii)
AEAKYAK-[BM]-DPSQSSELLSEAKKLNDSQAPK;

wherein [BM] is a CD69-binding motif as defined herein; and
xxiv) an amino acid sequence which has at least 89% identity to the sequence defined in xxiii).

Again, polypeptides comprising minor changes as compared to the above amino acid sequences, which do not largely affect the tertiary structure and the function of the polypeptide, also fall within the scope of the present disclosure. Thus, in some embodiments, sequence xviii), xx), xxii) or xxiv) may for example be at least 89%, such as at least 91%, such as at least 93%, such as at least 94%, such as at least 96%, such as at least 98% identical to a sequence defined by xvii), xix), xxi) and xxiii), respectively.

The terms “CD69-binding” and “binding affinity for CD69” as used in this specification refer to a property of a polypeptide which may be tested for example by ELISA or by the use of surface plasmon resonance (SPR) technology.

For example, as described in the examples below, CD69-binding affinity may be tested in an experiment in which CD69, or a fragment thereof, is immobilized on a sensor chip of a surface plasmon resonance (SPR) instrument, and the sample containing the polypeptide to be tested is passed over the chip. Alternatively, the polypeptide to be tested is immobilized on a sensor chip of the instrument, and a sample containing CD69, or a fragment thereof, is passed over the chip. The skilled person may then interpret the results obtained by such experiments to establish at least a qualitative measure of the binding affinity of the polypeptide for CD69. If a quantitative measure is desired, for example to determine a K_{D} value for the interaction, surface plasmon resonance methods may also be used. Binding values may for example be defined using a Biacore T200 instrument (Cytiva) or ProteOn XPR 36 (Bio-Rad) instrument. CD69 is suitably immobilized on a sensor chip of the instrument, and samples of the polypeptide whose affinity is to be determined are prepared by serial dilution and injected in random order. K_{D} values may then be calculated from the results using for example the 1:1 Langmuir binding model of the BIAevaluation 4.1 software, or other suitable software, provided by the instrument manufacturer.

The terms “albumin binding” and “binding affinity for albumin” as used in this disclosure refer to a property of a polypeptide which may be tested for example by the use of SPR technology in a Biacore instrument or ProteOn XPR36 instrument, in an analogous way to the example described above for CD69.

In one embodiment, the CD69-binding polypeptide is capable of binding to CD69 such that the K_{D} value of the interaction with CD69 is at most 1×10⁻⁶ M, such as at most 5×10⁻⁷ M, such as at most 1×10⁻⁷ M, such as at most 5×10⁻⁸ M, such as at most 1×10⁻⁸ M, such as at most 5×10⁻⁸ M.

The skilled person will understand that various modifications and/or additions can be made to a CD69-binding polypeptide according to any aspect disclosed herein in order to tailor the polypeptide to a specific application without departing from the scope of the present disclosure.

For example, in one embodiment, there is provided a CD69-binding polypeptide as described herein, which polypeptide has been extended by and/or comprises additional amino acids at the C terminus and/or N terminus. Such a polypeptide should be understood as a polypeptide having one or more additional amino acid residues at the very first and/or the very last position in the polypeptide chain. Thus, a CD69-binding polypeptide may comprise any suitable number of additional amino acid residues, for example at least one additional amino acid residue. Each additional amino acid residue may individually or collectively be added in order to, for example, improve and/or simplify production, purification, stabilization in vivo or in vitro, coupling or detection of the polypeptide. Such additional amino acid residues may comprise one or more amino acid residues added for the purpose of chemical coupling. One example of this is the addition of a cysteine residue. Additional amino acid residues may also provide a “tag” for purification or detection of the polypeptide, such as a hexahistidyl tag (“His tag” or “H₆ tag”), a (HisGlu)₃ tag (“HEHEHE tag”), a GGGC tag, a c-myc (“myc”) tag or a “FLAG” tag. Such a tag may for example enable interaction with antibodies specific to the tag, coupling with labels such as radioactive metal atoms, or immobilized metal affinity chromatography (IMAC) as the case may be. The skilled person is aware of such options and may make use of them without

In one embodiment, there is provided a CD69-binding polypeptide as described herein which comprises additional amino acids at the C-terminal and/or N-terminal end. For example, in one embodiment of the CD69-binding polypeptide as disclosed herein, it consists of any one of the sequences disclosed herein, having from 0 to 15 additional C-terminal and/or N-terminal residues, such as from 0 to 7 additional C-terminal and/or N-terminal residues. In one embodiment, the CD69-binding polypeptide consists of any one of the sequences disclosed herein, having from 0 to 15, such as from 0 to 4, such as 3 additional C-terminal residues. In one particular embodiment, the CD69-binding polypeptide as described herein comprises the additional C-terminal residues APK.

The further amino acids as discussed above may be coupled to the CD69-binding polypeptide by means of chemical conjugation (using known organic chemistry methods) or by any other means, such as expression of the CD69-binding polypeptide as a fusion protein or joined in any other fashion, either directly or via a linker, for example an amino acid linker.

A further polypeptide domain may moreover provide another CD69-binding moiety. Thus, in a further embodiment, there is provided a CD69-binding polypeptide in a multimeric form. Said multimer is understood to comprise at least two CD69-binding polypeptides as disclosed herein as monomer units, the amino acid sequences of which may be the same or different. Multimeric forms of the polypeptides may comprise a suitable number of domains, each having a CD69-binding motif, and each forming a monomer within the multimer. These domains may have the same amino acid sequence, but alternatively, they may have different amino acid sequences. In other words, the CD69-binding polypeptide of the invention may form homo- or heteromultimers, for example homo- or heterodimers. In one embodiment, there is provided a CD69-binding polypeptide, wherein said monomer units are covalently coupled together. In another embodiment, said CD69-binding polypeptide monomer units are expressed as a fusion protein. In one embodiment, there is provided a CD69-binding polypeptide in dimeric form. In one particular embodiment, said dimeric form is a homodimeric form. In another embodiment, said dimeric form is a heterodimeric form. For the sake of clarity, throughout this disclosure, the term “CD69-binding polypeptide” is used to encompass CD69-binding polypeptides in all forms, i.e. monomeric and multimeric forms.

The further amino acids as discussed above may for example comprise one or more further polypeptide domain(s). A further polypeptide domain may provide the CD69-binding dimer with another function, such as for example another binding function, or an enzymatic function, or a toxic function or a fluorescent signaling function, or combinations thereof.

Furthermore, it may be beneficial that the CD69-binding polypeptide as defined herein is part of a fusion protein or a conjugate comprising a second or further moieties. Second and further moiety/moieties of the fusion polypeptide or conjugate in such a protein may suitably have a desired biological activity.

Thus, in a second aspect of the present disclosure, there is provided a fusion protein or conjugate, comprising a first moiety consisting of a CD69-binding polypeptide according to the first aspect, and a second moiety consisting of a polypeptide having a desired biological activity. In another embodiment, said fusion protein or conjugate may additionally comprise further moieties, comprising desired biological activities that can be either the same as or different from the biological activity of the second moiety.

Non-limiting examples of a desired biological activity comprise a therapeutic activity, a binding activity and an enzymatic activity. In one embodiment, the second moiety having a desired biological activity is a therapeutically active polypeptide. In one embodiment, said second moiety is an immune response modifying agent.

In one embodiment of either the first or second aspect of the present disclosure, there is provided a CD69-binding polypeptide, fusion protein or conjugate which comprises an additional immune response modifying agent. Non-limiting examples of additional immune response modifying agents include immunomodulating agents or other anti-inflammatory agents.

Non-limiting examples of therapeutically active polypeptides are biomolecules, such as molecules selected from the group consisting of human endogenous enzymes, hormones, growth factors, chemokines, cytokines and lymphokines.

Non-limiting examples of binding activities are binding activities which increase the in vivo half-life of the fusion protein or conjugate, and binding activities which act to block a biological activity. One example of such a binding activity is a binding activity, which increases the in vivo half-life of a fusion protein or conjugate. In one embodiment of said fusion protein or conjugate, the in vivo half-life of said fusion protein or conjugate is longer than the in vivo half-life of the polypeptide having the desired biological activity per se. In one embodiment, said in vivo half-life is increased at least 10 times, such as at least 25 times, such as at least 50 times, such as at least 75 times, such as at least 100 times compared the in vivo half-life of the fusion protein or conjugate per se.

In one particular embodiment, the target for such binding activity is albumin, for example human serum albumin. Binding to albumin increases the in vivo half-life of said fusion protein or conjugate. In one embodiment, said albumin binding activity is provided by an albumin binding domain (ABD) of streptococcal protein G or a derivative thereof. Thus, said fusion protein may for example comprise a CD69-binding polypeptide in monomeric or multimeric form (such as a homodimeric or heterodimeric form) as defined herein and an albumin binding domain of streptococcal protein G or a derivative thereof. Suitable derivatives of the albumin binding domain of streptococcal protein G are known to the skilled person, and non-limiting examples are disclosed in WO2009/016043, WO2012/004384, WO2014/048977 and WO2015/091957.

In another embodiment, there is provided a fusion protein or a conjugate wherein said second moiety having a desired binding activity is a protein based on protein Z, derived from the B domain of protein A from Staphylococcus aureus, which has a binding affinity for a target other than CD69.

For example, said fusion protein or conjugate, comprising at least one further moiety, may comprise [CD69-binding polypeptide]-[albumin binding moiety]-[moiety with affinity for selected target]. It is to be understood that the three moieties in this example may be arranged in any order from the N- to the C-terminal of the polypeptide.

The skilled person is aware that the construction of a fusion protein often involves the use of linkers between the functional moieties to be fused, and there are different kinds of linkers with different properties, such as flexible amino acid linkers, rigid amino acid linkers and cleavable amino acid linkers. Linkers have been used to for example increase stability or improve folding of fusion proteins, to increase expression, improve biological activity, enable targeting and alter pharmacokinetics of fusion proteins. Thus, in one embodiment, the polypeptide according to any aspect disclosed herein further comprises at least one linker, such as at least one linker selected from flexible amino acid linkers, rigid amino acid linkers and cleavable amino acid linkers. In one embodiment, said linker is arranged between said CD69-binding polypeptide and a further polypeptide domain, such as between a CD69-binding domain as disclosed herein and an antibody or antigen binding fragment thereof (as described in further detail below). Flexible linkers are often used in the art when the joined domains require a certain degree of movement or interaction and may be particularly useful in some embodiments of the complex. Such linkers are generally composed of small, non-polar (for example G) or polar (for example S or T) amino acids. Some flexible linkers primarily consist of stretches of G and S residues, for example (GGGGS)ₚ. Adjusting the copy number “p” allows for optimization of linker in order to achieve appropriate separation between the functional moieties or to maintain necessary inter-moiety interaction. Apart from G and S linkers, other flexible linkers are known in the art, such as G and S linkers containing additional amino acid residues, such as T and A, to maintain flexibility, as well as polar amino acid residues to improve solubility. Additional non-limiting examples of linkers include GGGGSLVPRGSGGGGS, (GS)₃, (GS)₄, (GS)₈, GGSGGHMGSGG, GGSGGSGGSGG, GGSGG, GGSGGGGG, GGGSEGGGSEGGGSEGGG, AAGAATAA, GGGGG, GGSSG, GSGGGTGGGSG, GSGGGTGGGSG, GSGSGSGSGGSG, GSGGSGGSGGSGGS and GSGGSGSGGSGGSG, corresponding to SEQ ID NO:169-185, respectively, and GT. The skilled person is aware of other suitable linkers.

In one embodiment, said linker is a flexible linker comprising glycine (G), serine (S) and/or threonine (T) residues. In one embodiment, said linker has a general formula selected from (GₙSₘ)ₚ and (SₙGₘ)ₚ, wherein, independently, n=1-7, m=0-7, n+m 8 and p=1-7. In one embodiment, n=1-5. In one embodiment, m=0-5. In one embodiment, p=1-5. In a more specific embodiment, n=4, m=1 and p=1-4. In one embodiment, said linker is selected from the group consisting of S₄G, (S₄G)₃ and (S₄G)₄, corresponding to SEQ ID NO:186-188, respectively. In one embodiment, said linker is selected from the group consisting of G₄S and (G₄S)₃, corresponding to SEQ ID NO:189-190, respectively. In one particular embodiment, said linker is G₄S and in another embodiment said linker is (G₄S)₃.

With regard to the description above of fusion proteins or conjugates incorporating a CD69-binding polypeptide according to the disclosure, it is to be noted that the designation of first, second and further moieties is made for clarity reasons to distinguish between CD69-binding polypeptide or polypeptides according to the invention on the one hand, and moieties exhibiting other functions on the other hand. These designations are not intended to refer to the actual order of the different domains in the polypeptide chain of the fusion protein or conjugate. Similarly, the designations first and second monomer units are made for clarity reasons to distinguish between said units. Thus, for example, said first moiety (or monomer unit) may without restriction appear at the N-terminal end, in the middle, or at the C-terminal end of the fusion protein or conjugate.

The above aspects furthermore encompass polypeptides in which the CD69-binding polypeptide according to the first aspect or as comprised in a fusion protein or conjugate according to the second aspect further comprises a label, such as a label selected from the group consisting of fluorescent dyes and metals, chromophoric dyes, chemiluminescent compounds, bioluminescent proteins, enzymes, radionuclides, radioactive particles and pretargeting recognition tags. Such labels may for example be used for detection of the polypeptide and its target(s), e.g. CD69. For example, in some embodiments, such labeled polypeptide may for example be used for labeling, targeting and/or detecting cells and tissues which have a high expression of CD69, such as pancreatic tissues with a high infiltration of activated immune cells.

Indirect labeling of a Z variant polypeptide was recently shown using pretargeting recognition tags (Westerlund et al, 2015, Bioconjugate Chem 26:1724-1736). Similarly, the disclosure provides a CD69-binding polypeptide as described herein labeled with a pretargeting moiety, which may then be used for indirect labeling with a moiety complementary to the pretargeting moiety. When comprising a pretargeting moiety, a CD69-binding agent of the present disclosure is able to associate with a complementary pretargeting moiety, and such complementary pretargeting moiety may then comprise or be attached to a suitable radionuclide. The skilled person is aware of suitable radionuclides for therapeutic, diagnostic and/or prognostic purposes. Such a radionuclide may be chelated to said complementary pretargeting moiety via a chelating environment as generally described for the CD69-binding agent below.

In embodiments in which the polypeptide, fusion protein or conjugate is labeled, directly or indirectly (e.g. via pretargeting as described above), with an imaging agent (e.g. radioactive agent), measuring the amount of labeled polypeptide present in a tissue, such as pancreas, may be done using imaging equipment, such as through acquiring radioactivity counts or images of radiation density, or derivatives thereof such as radiation concentration. Non-limiting examples of radionuclides, suitable either for direct labeling of the CD69-binding agent according to any aspect disclosed herein or for indirect labeling by labeling of a complementary pretargeting moiety, include ⁶⁸Ga, ^{110m}In, ¹⁸F, ⁴⁵Ti, ⁴⁴Sc, ⁶¹Cu, ⁶⁶Ga, ⁶⁴Cu, ⁵⁵Co, ⁷²As, ⁸⁶Y, ⁸⁹Zr, ¹²⁴I, ⁷⁶Br, ¹¹¹In, ^{99m}Tc, ¹²³I, ¹³¹I and ⁶⁷Ga.

In one embodiment, the imaging equipment used in such measurements is positron emission tomography (PET) equipment, in which case the radionuclide is selected such that it is suitable for PET. The skilled person is aware of radionuclides suitable for use with PET. For example, a PET radionuclide is selected from the group consisting of ⁶⁸Ga, ^{110m}In, ¹⁸F, ⁴⁵Ti, ⁴⁴Sc, ⁶¹Cu, ⁶⁶Ga, ⁶⁴Cu, ⁵⁵Co, ⁷²As, ⁸⁶Y, ⁸⁹Zr, ¹²⁴I and ⁷⁶Br.

In another embodiment, the imaging equipment used is single-photon emission computed tomography (SPECT) equipment, in which case the radionuclide is selected such that it is suitable for SPECT. The skilled person is aware of radionuclides suitable for use with SPECT. For example, a SPECT radionuclide is selected from the group consisting of ¹¹¹In, ^{99m}Tc, ¹²³I, ¹³¹I and ⁶⁷Ga.

Thus, in one embodiment there is provided a CD69-binding polypeptide, fusion protein or complex as described herein, which comprises a direct or indirect radionuclide label, such as a radionuclide selected from the group consisting of ⁶⁸Ga, ^{110m}In, ¹⁸F, ⁴⁵Ti, ⁴⁴Sc, ⁶¹Cu, ⁶⁶Ga, ⁶⁴Cu, ⁵⁵Co, ⁷²As, ⁸⁶Y, ⁸⁹Zr, ¹²⁴I, ⁷⁶Br, ¹¹¹In, ^{99m}Tc, ¹²³I, ¹³¹I, ⁶⁷Ga such as the group consisting of ⁶⁸Ga, ^{110m}In, ¹⁸F, ⁴⁵Ti, ⁴⁴Sc, ⁶¹Cu, ⁶⁶Ga, ⁶⁴Cu, ⁵⁵Cu, ⁷²As, ⁸⁶Y, ⁸⁹Zr, ¹²⁴I and ⁷⁶Br, such as ¹⁸F.

In some embodiments, the labeled CD69-binding polypeptide is present as a moiety in a fusion protein or conjugate also comprising a second moiety having a desired biological activity. The label may in some instances be coupled only to the CD69-binding polypeptide, and in some instances both to the CD69-binding polypeptide and to the second moiety of the fusion protein or conjugate. Furthermore, it is also possible that the label may be coupled to a second moiety only and not to the CD69-binding moiety. Hence, in yet another embodiment, there is provided a CD69-binding polypeptide comprising a second moiety, wherein said label is coupled to the second moiety only.

When reference is made to a labeled polypeptide, this should be understood as a reference to all aspects of polypeptides as described herein, including CD69-binding polypeptides, fusion proteins and conjugates comprising a CD69-binding polypeptide. Thus, a labeled polypeptide may contain only the CD69-binding polypeptide and e.g. a radionuclide, which may be chelated or covalently coupled to the CD69-binding polypeptide, or contain the CD69-binding polypeptide, a radionuclide and a second moiety such as a small molecule having a desired biological activity, for example a therapeutic efficacy. A labeled polypeptide may contain a CD69-binding polypeptide in heterodimeric form and e.g. a radionuclide, which may be chelated or covalently coupled to the CD69-binding polypeptide, or contain the CD69-binding polypeptide in heterodimeric form, a therapeutic radionuclide and a second moiety such as a small molecule having a desired biological activity, for example a therapeutic efficacy. The skilled person is aware of other possible variants.

In embodiments where the CD69-binding polypeptide, fusion protein or conjugate is radiolabeled, such a radiolabeled polypeptide may comprise a radionuclide, such as selected from ⁶⁸Ga and ¹⁸F suitable for imaging.

A majority of radionuclides have a metallic nature and metals are typically incapable of forming stable covalent bonds with elements presented in proteins and peptides. For this reason, labeling of proteins and peptides with radioactive metals is performed with the use of chelators, i.e. multidentate ligands, which form non-covalent compounds, called chelates, with the metal ions. In an embodiment of the CD69-binding polypeptide, fusion protein or conjugate, the incorporation of a radionuclide is enabled through the provision of a chelating environment, through which the radionuclide may be coordinated, chelated or complexed to the polypeptide. One example of a chelator is the polyaminopolycarboxylate type of chelator. Two classes of such polyaminopolycarboxylate chelators can be distinguished: macrocyclic and acyclic chelators.

In one embodiment, the CD69-binding polypeptide, fusion protein or conjugate comprises a chelating environment provided by a polyaminopolycarboxylate chelator conjugated to the CD69-binding polypeptide via a thiol group of a cysteine residue or an epsilon amine group of a lysine residue.

The most commonly used macrocyclic chelators for radioisotopes of indium, gallium, yttrium, bismuth, radioactinides and radiolanthanides are different derivatives of DOTA (1,4,7,10-tetraazacyclododecane-1,4,7,10-tetraacetic acid). In one embodiment, a chelating environment of the CD69-binding polypeptide, CD69-binding polypeptide in heterodimeric form, fusion protein or conjugate is provided by DOTA or a derivative thereof. More specifically, in one embodiment, a chelating polypeptide encompassed by the present disclosure is obtained by reacting the DOTA derivative 1,4,7,10-tetraazacyclododecane-1,4,7-tris-acetic acid-10-maleimidoethylacetamide (maleimidomonoamide-DOTA) with said polypeptide. In one embodiment, a chelating polypeptide encompassed by the present disclosure is obtained by reacting the DOTA derivative DOTAGA (2,2′,2″-(10-(2,6-dioxotetrahydro-2H-pyran-3-yl)-1,4,7,10-tetraazacyclododecane-1,4,7-triyl)triacetic acid) with said polypeptide. Additionally, 1,4,7-triazacyclononane-1,4,7-triacetic acid (NOTA) and derivatives thereof may be used as chelators. Hence, in one embodiment, a chelating environment of the CD69-binding polypeptide, CD69-binding polypeptide in heterodimeric form, fusion protein or conjugate is provided by NOTA or a derivative thereof. In one embodiment, a chelating polypeptide encompassed by the present disclosure is obtained by reacting the NOTA derivative NODAGA (2,2′-(7-(1-carboxy-4-((2,5-dioxopyrrolidin-1-yl)oxy)-4-oxobutyl)-1,4,7-triazonane-1,4-diyl)diacetic acid) with said polypeptide. The most commonly used acyclic polyaminopolycarboxylate chelators are different derivatives of DTPA (diethylenetriamine-pentaacetic acid). Hence, polypeptides having a chelating environment provided by diethylenetriaminepentaacetic acid or derivatives thereof are also encompassed by the present disclosure.

In further aspects of the present disclosure, there is provided a polynucleotide encoding a CD69-binding polypeptide or fusion protein as described herein; an expression vector comprising said polynucleotide; and a host cell comprising said expression vector.

Also encompassed by this disclosure is a method of producing a CD69-binding polypeptide or fusion protein as described above, comprising culturing said host cell under conditions permissive of expression of said polypeptide from its expression vector, and isolating the polypeptide.

The CD69-binding polypeptide or fusion protein of the present disclosure may alternatively be produced by non-biological peptide synthesis using amino acids and/or amino acid derivatives having protected reactive side-chains, the non-biological peptide synthesis comprising
step-wise coupling of the amino acids and/or the amino acid derivatives to form a polypeptide or fusion protein as described herein having protected reactive side-chains,
removal of the protecting groups from the reactive side-chains, and
folding of the polypeptide or fusion protein in aqueous solution.

It should be understood that the CD69-binding polypeptide according to the present disclosure may be useful as a therapeutic, diagnostic and/or prognostic agent in its own right or as a means for targeting other therapeutic, diagnostic and/or prognostic agents to CD69-expressing cells and tissues.

Thus, in another aspect, there is provided a composition comprising a CD69-binding polypeptide, fusion protein or conjugate as described herein and at least one pharmaceutically acceptable excipient or carrier. In one embodiment, said composition further comprises at least one additional active agent, such as at least two additional active agents, such as at least three additional active agents. Non-limiting examples of additional active agents that may prove useful in such combination are immune response modifying agents as described herein.

The small size and robustness of the CD69-binding polypeptides of the present disclosure confer several advantages over larger molecules, such as conventional monoclonal antibody-based therapies. Such advantages include advantages in formulation, modes of administration, such as alternative routes of administration, administration at higher doses than antibodies and absence of Fc-mediated side effects.

In comparison to using other immune cell markers than CD69 for similar purposes, it is an advantage of the present disclosure that CD69 is expressed on most activated immune cells, especially early in the activation process. This is advantageous, because the number of T-cells alone infiltrating tissue (e.g. pancreas) is low and thus difficult to detect by imaging. A general marker for activated immune cells as CD69 is thus more sensitive for detecting early sub-clinical immune responses. Furthermore, in contrast to approaches that target either CD4 or CD8 for imaging, background binding to resting immune cells is negligible, as demonstrated by the in vivo studies presented in the appended Examples.

The agents of the present disclosure are contemplated for oral, topical, intravenous, intraperitoneal, subcutaneous, pulmonary, transdermal, intramuscular, intranasal, buccal, sublingual or suppository administration. In particular for diagnostic imaging applications, administration via the intravenous or subcutaneous route is preferred.

Also, many diseases and disorders, such as autoimmune-related disorders, for example type 1 diabetes (T1D), are associated with more than one factor. Thus, a polypeptide as defined herein confers the advantageous option of targeting an additional antigen together with CD69.

In another aspect of the present disclosure, there is provided a CD69-binding polypeptide, fusion protein, conjugate or composition as described herein for use as a medicament, a prognostic agent and/or a diagnostic agent. In one embodiment, there is provided a CD69-binding polypeptide, fusion protein, conjugate or composition for use in the treatment, diagnosis or prognosis of a CD69-related disorder.

In one embodiment, said CD69-binding polypeptide, fusion protein, conjugate or composition is provided for use as a medicament and/or as a diagnostic agent in vivo and/or as a prognostic agent in vivo.

In one embodiment, there is provided a CD69-binding polypeptide, fusion protein, conjugate or composition for use in the treatment of a CD69-related disorder.

In one embodiment, there is provided a CD69-binding polypeptide, fusion protein, conjugate or composition for use in the in vivo diagnosis of a CD69-related disorder.

In one embodiment, there is provided a CD69-binding polypeptide, fusion protein, conjugate or composition for use in the in vivo prognosis of a CD69-related disorder.

As used herein, the term “CD69-related disorder” refers to any disorder, disease or condition in which CD69 signaling and/or expression plays a role. Examples of such CD69-related disorder include inflammatory diseases, including autoinflammatory diseases, for example type 1 diabetes (T1D).

It is to be understood that said CD69-binding polypeptide, fusion protein, conjugate or composition may be used as the sole therapeutic, diagnostic or prognostic agent, or as a companion therapeutic, diagnostic and/or prognostic agent in a combination treatment, combination diagnostic method or combination prognostic method.

As such, in one embodiment of a therapeutic use, it is beneficial to administer a therapeutically effective amount of a CD69-binding polypeptide, fusion protein, conjugate or composition as described herein together with at least one second drug substance, such as an immune response modifying agent.

In a related aspect, there is provided a method of treatment of a CD69-related disorder, comprising administering to a subject in need thereof an effective amount of a CD69-binding polypeptide, fusion protein, conjugate or composition as described herein. The skilled person will appreciate that any description in relation to the CD69-binding polypeptide, fusion protein, conjugate or composition as described herein for use in treatment of a disease or disorder is equally relevant for the related therapeutic method. For the sake of brevity, such description will not be repeated here.

In another aspect of the present disclosure, there is provided a method, such as an in vitro method, of detecting CD69, comprising providing a sample suspected to contain CD69, contacting said sample with a CD69-binding polypeptide, fusion protein, conjugate or composition as described herein, and detecting the binding of the CD69-binding polypeptide, fusion protein, conjugate or composition to indicate the presence of CD69 in the sample.

In one embodiment, said method further comprises an intermediate washing step for removing non-bound polypeptide, fusion protein, conjugate or composition, after contacting the sample.

In another embodiment, said method is a diagnostic or prognostic method for determining the presence of CD69 in a subject, the method comprising the steps:
a) contacting the subject, or a sample isolated from the subject, with a CD69-binding polypeptide, fusion protein, conjugate or composition as described herein, and
b) obtaining a value corresponding to the amount of the CD69-binding polypeptide, fusion protein, conjugate or composition that has bound in said subject or to said sample.

In one embodiment, said method further comprises an intermediate washing step for removing non-bound polypeptide, fusion protein, conjugate or composition, after contacting the subject or sample and before obtaining a value.

In one embodiment of this diagnostic or prognostic method, said CD69-binding polypeptide, fusion protein or conjugate comprises a pretargeting moiety as described herein, and the contacting step a) of the method further comprises contacting the subject with a complementary pretargeting moiety labeled with a detectable label, such as a radionuclide label.

In one embodiment, said method further comprises a step of comparing said value to a reference. Said reference may be by a numerical value, a threshold or a visual indicator, for example based on a color reaction. The skilled person will appreciate that different ways of comparison to a reference are known in the art and may be suitable for use.

In one embodiment of such a method, said subject is a mammalian subject, such as a human subject. In one embodiment, said method is performed in vivo. In another embodiment, said method is performed in vitro.

In one embodiment, the diagnostic or prognostic method is a method for medical imaging in vivo. Such a method comprises the systemic administration of a CD69-binding entity as disclosed herein (i.e. the polypeptide per se, or the fusion protein, conjugate or composition containing it) to a mammalian subject. The CD69-binding entity is directly or indirectly labelled, with a label comprising a radionuclide suitable for medical imaging (see above for a list of contemplated radionuclides). Furthermore, the method for medical imaging comprises obtaining one or more images of at least a part of the subject's body using a medical imaging instrument, said image(s) indicating the presence of the radionuclide inside the body. In one particular embodiment, said part of the subject's body is the pancreas.

The skilled person will appreciate that any description in relation to the CD69-binding polypeptide, fusion protein, conjugate or composition as described herein for use in diagnosis and/or prognosis of a disease or disorder is equally relevant for the related diagnostic or prognostic method in vivo. For the sake of brevity, such description will not be repeated here.

While the invention has been described with reference to various exemplary aspects and embodiments, it will be understood by those skilled in the art that various changes may be made, and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment contemplated, but that the invention will include all embodiments falling within the scope of the appended claims.

### BRIEF DESCRIPTION OF THE FIGURES

FIG. 1 is a listing of the amino acid sequences of CD69-binding polypeptides selected in Example 2 (SEQ ID NO:78) and Example 5 (SEQ ID NO:73-77 and 79-144); site-directed scaffold mutants of these CD69-binding polypeptides as characterized and studied in Examples 3-4 (SEQ ID NO:6) and Example 6 (SEQ ID NO:1-72); various constructs incorporating said CD69-binding polypeptides and produced as described in Examples 3, 4 and 6 (SEQ ID NO:145-162); control polypeptide with affinity for an irrelevant target (ZTAQ, SEQ ID NO:163); the albumin binding polypeptide ABD (SEQ ID NO:164); extracellular domain of human CD69 (SEQ ID NO:165); extracellular domain of murine CD69 (SEQ ID NO:166) and human serum albumin (SEQ ID NO:167). The deduced CD69-binding motifs (BM) extend from residue 8 to residue 36 in sequences with SEQ ID NO:1-144. The amino acid sequences of the 49 amino acid residues long polypeptides (BMod) predicted to constitute the complete three-helix bundle within each of these Z variants extend from residue 7 to residue 55.

FIGS. 2A-C are dot plots showing selection from an E. coli-displayed library as described in Example 2 after one round of MACS, followed by one (A), two (B) and three (C) rounds of FACS with the target hCD69 and human serum albumin (HSA) labelled with two different fluorophores. The X axis shows fluorescence intensity corresponding to surface expression level as measured by incubation with fluorescently labelled HSA. The Y axis shows fluorescence intensity corresponding to binding of labeled target. Gates used for FACS are shown in the dot plots, and the percentage of the population within respective gate is indicated.

FIG. 3 is a photograph of SDS-PAGE analysis of IMAC-purified H₆-ZC69006-ABD fusion (lane 1), H₆-ZC69006 (lane 2), and H₆-ZC69006-Cys (lane 3).

FIG. 4 is a series of SPR sensorgrams showing the binding of H₆-ZC69006-ABD to (A) human CD69, (B) murine CD69 and (C) human serum albumin at different concentrations as indicated.

FIG. 5A shows a circular dichroism spectrum of H₆-ZC69006 at 20° C. before and after heat-induced denaturation. FIG. 5B shows the result of thermal stability analysis of H₆-ZC69006 using circular dichroism spectroscopy.

FIG. 6 is a diagram showing the magnitude of binding of ¹¹¹In-DOTA-ZC69006 correlated to the percentage of CD69 positive cells in each batch. Human peripheral blood monocytes and mouse splenic cells were either activated by incubation with anti-CD3 antibody (“Activated”) or resting (“Non-activated”).

FIG. 7 shows images from representative coronal SPECT and CT of ¹¹¹In-DOTA-ZC69006 in rat, demonstrating targeting of lymph nodes (Ln), renal excretion and kidney cortex trapping of radionuclide (Ki) as well as low background binding in e.g. liver (Li) (A), and of negative control ¹¹¹In-DOTA-ZTAQ, demonstrating that it did not exhibit any targeting of lymph nodes in any rat at any time point while otherwise demonstrating a similar biodistribution (B). The figure also shows a bar diagram of the dynamic biodistribution of ¹¹¹In-DOTA-ZC69006 in rats (n=3) quantified as SUV (C) as well as an image showing accumulation of ¹¹¹In-DOTA-ZC69006 at the site of an islet allograft (Ig) in mouse (D). The inferior part of the kidneys (Ki) and the bladder (BI) are also indicated.

FIG. 8 shows dot plots of the E. coli-displayed affinity maturation library during each of four consecutive cycles of FACS (A)-(D). X-axes: fluorescence intensity corresponding to surface expression level as measured by incubation with fluorescently labeled HSA. Y-axes: fluorescence intensity corresponding to labeled CD69 binding. Gates used for FACS are indicated.

FIG. 9 is a representative series of SPR sensorgrams showing the binding of H₆-ZC69002-ABD to (A) human CD69, (B) murine CD69 and (C) human serum albumin at different concentrations as indicated.

FIG. 10A shows a representative circular dichroism spectrum of H₆-ZC69001 at 20° C. before and after heat-induced denaturation. FIG. 10B shows the result of thermal stability analysis of H₆-ZC69001 using circular dichroism spectroscopy.

FIG. 11 shows representative UV (top) and radiodetector (bottom) HPLC chromatograms following radiolabeling of Z variant ZC69001.

FIG. 12 are bar diagrams showing uptake in kidney (left), liver (middle) and muscle (right) of five different indium-111 radiolabeled Z variants, measured from SPECT/CT images (n=3 rats each). Black bars: ¹¹¹In-DOTA-ZC69006. Bars with vertical lines: ¹¹¹In-DOTA-ZC69001. Grey bars: ¹¹¹In-DOTA-ZC69002. Bars with square pattern: ¹¹¹In-DOTA-ZC69003. White bars: ¹¹¹In-DOTA-ZC69005.

FIG. 13 shows the dynamic uptake over time in kidney, liver, heart left ventricle, lungs and muscle tissue (average of n=3 rats each) for ¹¹¹In-DOTA-ZC69006 (A), ¹¹¹In-DOTA-ZC69001 (B), ¹¹¹In-DOTA-ZC69002 (C), ¹¹¹In-DOTA-ZC69003 (D) and ¹¹¹In-DOTA-ZC69005 (E).

FIG. 14 shows representative images of accumulation of ¹¹¹In-DOTA-ZC69002 in lymph nodes as assessed by SPECT/CT. Ex vivo autoradiograms (A) of SPECT positive lymph nodes (Ln), as well as surrounding adipose tissue (Ad) was in agreement with the in vivo images (B).

FIG. 15 is a graph showing the binding of ¹⁸F-TZ-ZC69001 in CD69 transfected CHO-K1 cells at 3 nM (white bars) and 30 nM (black bars). Binding was assessed with ¹⁸F-TZ-ZC69001 alone (“Total”) or following preincubation with excess ZC69001-Cys (“Blocked”).

FIG. 16 is a graph showing the binding of ⁶⁸Ga-DOTA-ZC69001 in the hind leg joints in an induced model of progressive rheumatoid arthritis. The PET tracer uptake is shown as black filled circles and is given on the left y-axis. The “RA score”, indicating grade of swelling and inflammation by clinical examination, is shown as open circles and is given on the right y-axis.

FIG. 17 is a graph showing binding of ⁶⁸Ga-DOTA-ZC69001 in the lung of pig with induced lung injury and inflammation (A), ⁶⁸Ga-DOTA-ZC69001 lung binding in a control pig (B) as well as binding of non-CD69-binding control peptide ⁶⁸Ga-DOTA-ZAM106 in a pig with lung inflammation (C).

### EXAMPLES

### Summary

The following Examples disclose the development of novel Z variant molecules targeted to CD69 based on autotransporter-mediated E. coli display and affinity maturation. The Examples further describe the characterization of CD69-binding polypeptides and demonstrate in vitro functionality of said polypeptides.

### Example 1

### Generation of a Z Variant Library for Display on E. coli Cells

A 121 nucleotide long, randomized oligonucleotide encoding helix 1 and 2 of the Z variant library was designed and synthesized (Ella Biotech GmbH, Martinsried, Germany). Randomized positions were designed to have the codon distribution shown in Table 2. Note in particular that only five amino acids were allowed in position 31 of the full length sequence.

TABLE 2
Design of Z variant library for primary selection

| Position in the Z | | |
|---|---|---|
| variant sequence | Permitted amino acid residues | Proportions |
| 9 | A, D, E, F, G, H, I, K, L, M, N, Q, R, S, T, V, W, Y | 5.6% |
| 10 | A, D, E, F, G, H, I, K, L, M, N, Q, R, S, T, V, W, Y | 5.6% |
| 11 | A, D, E, F, G, H, I, K, L, M, N, Q, R, S, T, V, W, Y | 5.6% |
| 13 | A, D, E, F, G, H, I, K, L, M, N, Q, R, S, T, V, W, Y | 5.6% |
| 14 | A, D, E, F, G, H, I, K, L, M, N, Q, R, S, T, V, W, Y | 5.6% |
| 17 | A, D, E, F, G, H, I, K, L, M, N, Q, R, S, T, V, W, Y | 5.6% |
| 18 | A, D, E, F, G, H, I, K, L, M, N, Q, R, S, T, V, W, Y | 5.6% |
| 24 | A, D, E, F, G, H, I, K, L, M, N, Q, R, S, T, V, W, Y | 5.6% |
| 25 | A, D, E, F, G, H, I, K, L, M, N, Q, R, S, T, V, W, Y | 5.6% |
| 27 | A, D, E, F, G, H, I, K, L, M, N, Q, R, S, T, V, W, Y | 5.6% |
| 28 | A, D, E, F, G, H, I, K, L, M, N, Q, R, S, T, V, W, Y | 5.6% |
| 31 | D, H, I, K, Y | I: 60%; D: 10%; H: 10%; |
| | | K: 10%; Y: 10% |
| 32 | A, D, E, F, G, H, I, K, L, M, N, Q, R, S, T, V, W, Y | 5.6% |
| 35 | A, D, E, F, G, H, I, K, L, M, N, Q, R, S, T, V, W, Y | 5.6% |

The oligo was amplified in eight rounds of PCR. Ligation of the insert into the vector was done by adding a threefold molar excess of insert over vector and T4 DNA ligase.

The ligated vector was electroporated using 40 separate reactions into E. coli BL-21 cells. Samples of the total library were diluted and spread on agar plates, and the library size was estimated to be around 2.4×10⁹ variants. To validate the library, 192 separate clones picked at random were sequenced, finding only unique clones and no observed errors. The validated E. coli library was stored at −80° C.

### Example 2

### Selection of a CD69-Binding Z Variant Using E. coli Display

### Aim

From the naïve Z variant library prepared as described in Example 1, Z variants with affinity for human CD69 were selected using a combination of magnetic-assisted cell sorting (MACS) and fluorescence-activated cell sorting (FACS) as previously described (Andersson et al (2018), supra). The MACS step is used to reduce the library size to make FACS selection feasible.

### Methods

Biotinylation of hCD69: Biotinylation of recombinant extracellular domain of human CD69 (SEQ ID NO:165; #8468-CD-025, R&D Systems), here denoted hCD69, was performed using EZ-Link NBS-Biotin (N-hydroxysuccinimidobiotin) (#20217, Thermo Scientific) according to the supplier's recommendations. The protein buffer was changed to PBS (10 mM phosphate, 137 mM NaCl, 2.68 mM KCl, pH 7.4) by dialysis twice, first against 2 l PBS and then against 3 l PBS.

MACS selection: 500 μl of streptavidin-coated Dynabeads (DYNABEADS® M-280 Streptavidin, Thermo Scientific) were washed twice in 1×PBSP (phosphate-buffered saline with 0.1% Pluronic F108 NF Prill Poloxamer 338 surfactant). The beads were resuspended in 100 nM of biotinylated hCD69 and incubated for 1 h at room temperature (RT) in a rotamixer. 4×10¹⁰ E. coli cells were pelleted and washed in 1×PBSP and negative selection was carried out by adding 250 μl of Dynabeads without biotinylated hCD69, followed by incubation for 20 min at RT in a rotamixer and subsequent removal of magnetic beads. The target-coated beads were added to the cell suspension and incubated for 60 min at RT in a rotamixer. The magnetic beads were washed three times using 30 ml of ice-cold PBSP and thereafter inoculated to 50 ml of LB medium containing chloramphenicol. The culture was incubated overnight at 37° C. and an aliquot was spread on agar plates containing chloramphenicol for estimation of enrichment.

FACS selection: Induced recombinant E. coli cells were washed with 1×PBSP. Cells were resuspended in PBSP containing biotinylated hCD69. The mix was incubated on a rotamixer at RT for 1 h, washed with ice-cold PBSP, and resuspended in 150 nM human serum albumin (HSA)-Alexa 647 conjugate and 0.5 μg/ml streptavidin conjugated with R-phycoerythrin (SAPE; Invitrogen) or neutravidin conjugated with Oregon Green 488 (NAOG; Life Technologies), followed by incubation on ice for 30 min. The cells were subsequently washed with ice-cold PBSP and resuspended in ice-cold PBSP for sorting in a MoFlo Astrios EQ flow cytometer (Beckman Coulter) or analysis in a Gallios flow cytometer (Beckman Coulter). The E. coli library was sorted in a MoFlo Astrios EQ cell sorter (Beckman Coulter). Bacteria were sorted into a 1.5 ml tube containing LB medium and chloramphenicol. The sorted cells were incubated for 1 h on rotamixer at 37° C. and thereafter inoculated to 50 ml LB medium with chloramphenicol for overnight cultivation.

### Results

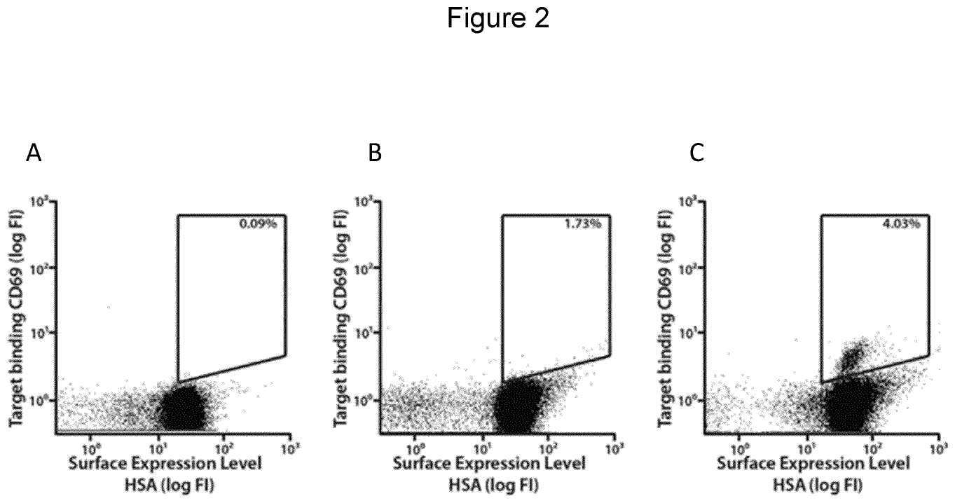

The MACS step reduced the library size approximately thousand-fold, from 2.4×10⁹ members to approximately 2.4×10⁶ members. The reduced library was then enriched in three consecutive rounds of FACS (FIG. 2A-C). A successful enrichment of potential CD69-binding Z variants displayed on the E. coli cells was observed as a “cloud” within the sorting gate in FIG. 2C. DNA sequencing of the Z variants displayed on E. coli was performed on 100 random bacterial clones. The same sequence, SEQ ID NO:78, was identified in all clones. This indicates a significant selection convergence towards this particular Z variant, which was denoted ZC69078.

### Example 3

### Biochemical Characterization of CD69-Binding Z Variant

When the CD69-binding Z variant identified as described in Example 2 was expressed recombinantly, the yield was lower than that which has been generally observed for other Z variants. Using homology alignment against previously reported Z variant domains, five amino acid replacements in the scaffold regions of helix three were identified and mutated. These substitution mutations in ZC69078 were S42A, E43N, S46A, Q50K and S54A, and are all outside of the posited binding surface of the three-helix domain polypeptide. The resulting mutated Z variant, having the amino acid sequence SEQ ID NO:6, is denoted ZC69006 herein. The expression yield for ZC69006 was at least 50-fold higher than that of ZC69078. Note that no mutations have been made to the CD69 binding motif of ZC69078, so ZC69078 and ZC69006 share the same binding motif sequence.

### Materials and Methods

Protein production: The gene encoding ZC69006 was subcloned into three different E. coli expression vectors based on pET22b (GenScript Biotech Corp) under control of a T7 promoter. All constructs had an N-terminal hexahistidine tag incorporated in the sequence MGSS-H₆-YYLE, and contained the gene encoding ZC69006 followed by the dipeptide -VD, introduced for cloning purposes. One construct had an additional C-terminal cysteine, and in another, ZC69006 was followed by a (G₄S)₃ linker and then an albumin binding domain (ABD) derived from streptococcal Protein G and having the amino acid sequence SEQ ID NO:164. The three respective expression products were denoted H₆-ZC69006 (SEQ ID NO:160), H₆-ZC69006-Cys (SEQ ID NO:161) and H₆-ZC69006-ABD (SEQ ID NO:162). The ligated vector was transformed into E. coli BL21(DE3) cells (Merck) for expression using standard protocols. The recombinant proteins were purified using HisPur Cobalt Resin (#89966, Thermo Scientific) according to the manufacturer's instructions.

SPR analysis: Human serum albumin (SEQ ID NO:167, HSA; #A3782, Sigma), extracellular domain from human CD69 (SEQ ID NO:165, hCD69; #8468-CD-025, R&D Systems) and extracellular domain from murine CD69 (SEQ ID NO:166, mCD69; #8469-CD-025, R&D Systems), were each diluted in 10 mM NaAc, pH 4.5 and immobilized on CM5 chip surfaces using EDC/NHS coupling chemistry for use as immobilized targets in a Biacore T200 instrument (GE Healthcare). The surfaces were inactivated using ethanolamine prior to binding studies. A first screening was performed by injecting 100 nM of H₆-ZC69006-ABD over the respective immobilized targets for 120 s, followed by running buffer for 300 s before regeneration of the surfaces. ZC69006 in fusion with ABD was first injected for non-covalent and directed capture on immobilized HSA, followed by injection of respective target molecule. Either 10 mM HCl or 10 mM glycine-HCl pH 2.5 were used for regeneration in the experiments.

Circular dichroism spectroscopy: Thermal stability and refolding after heat-induced denaturation was measured for H₆-ZC69006 using circular dichroism spectroscopy. All measurements were performed on a Chirascan™ instrument (Applied Photophysics Ltd, Surrey, UK). The thermal stability was determined by following the ellipticity at 221 nm during variable temperature measurements (5° C./min from 20° C. to 100° C.). After the heat-induced denaturation, the samples were cooled to RT and left for 15 min before measuring ellipticity at 20° C. from 195 nm to 260 nm in five replicates.

DOTA conjugation of H₆-ZC69006-Cys: After freeze-drying, H₆-ZC69006-Cys was resuspended in PBS supplemented with an equimolar amount of tris-(2-carboxyethyl)phosphine (TCEP) to 1 mg/ml (118 mM) and incubated for 20 min at 37° C. Following the incubation with TCEP, a 10-fold molar excess of 1,4,7,10-tetraazacyclododecane-1,4,7,10-tetraacetic acid (DOTA) maleimide was added and the sample was incubated at 37° C. for 3 h. The progression of the conjugation was monitored by MALDI.

HPLC purification: The DOTA-conjugated H₆-ZC69006-Cys was diluted using acetonitrile (ACN) with 0.1% trifluoroacetic acid (TFA) to a final concentration of 20% ACN, and then sterile filtered through a 0.2 μm filter prior to injecting into HPLC for purification on a Semi-Prep C18 column. A gradient from 20% to 56% ACN at 2.5 ml/min over 30 min was used. 500 μl fractions were collected for the peaks, and subsequently analyzed by MALDI.

Mass spectrometry (MS): MALDI was used to verify DOTA conjugation during the conjugation as well as after HPLC purification. All samples were analyzed on a 4800 MALDI (Applied Biosystems). 1 μl sample was mixed with 1 μl α-cyano-4-hydroxy-cinnamic acid matrix (Bruker Daltonics), added to the MALDI plate and substituted with another μl of matrix before loading the plate in the MS.

### Results

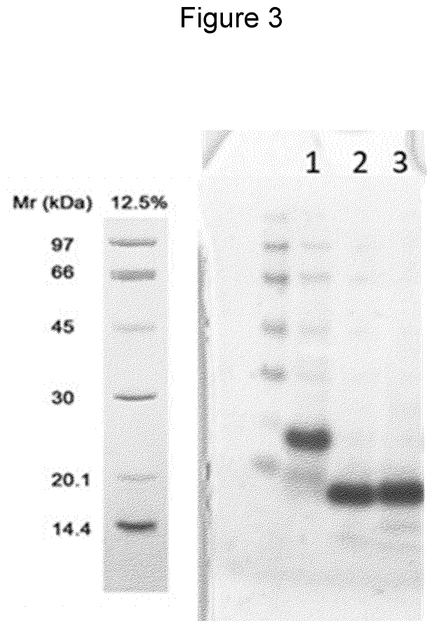

Protein production: The Z variant molecules H₆-ZC69006, H₆-ZC69006-Cys and H₆-ZC69006-ABD were purified after expression to high purity using IMAC, as evidenced by SDS-PAGE (FIG. 3).

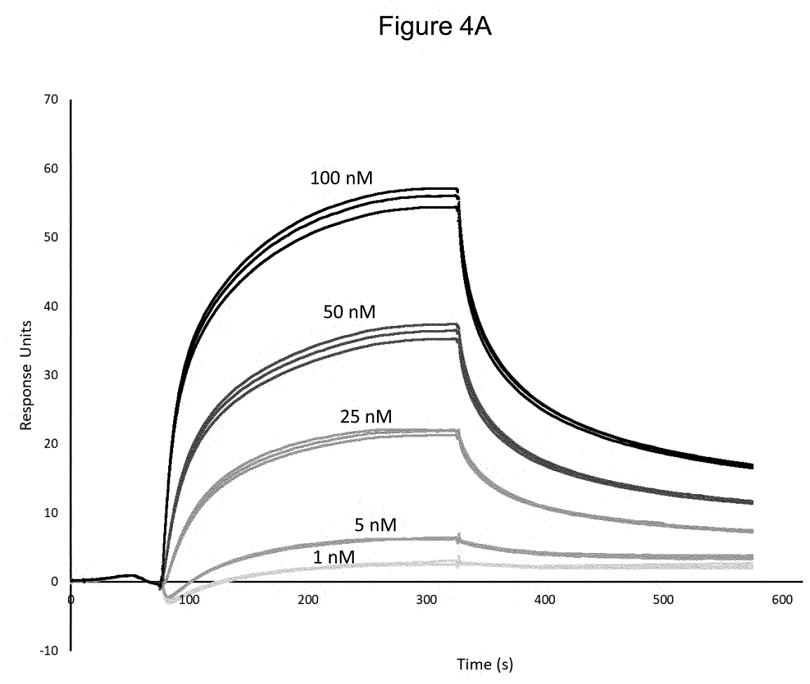

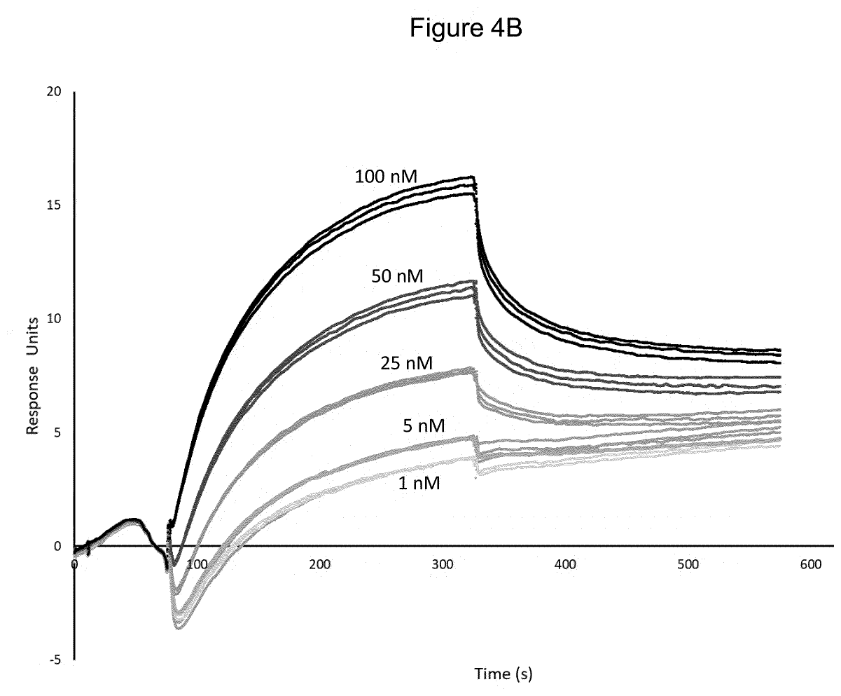

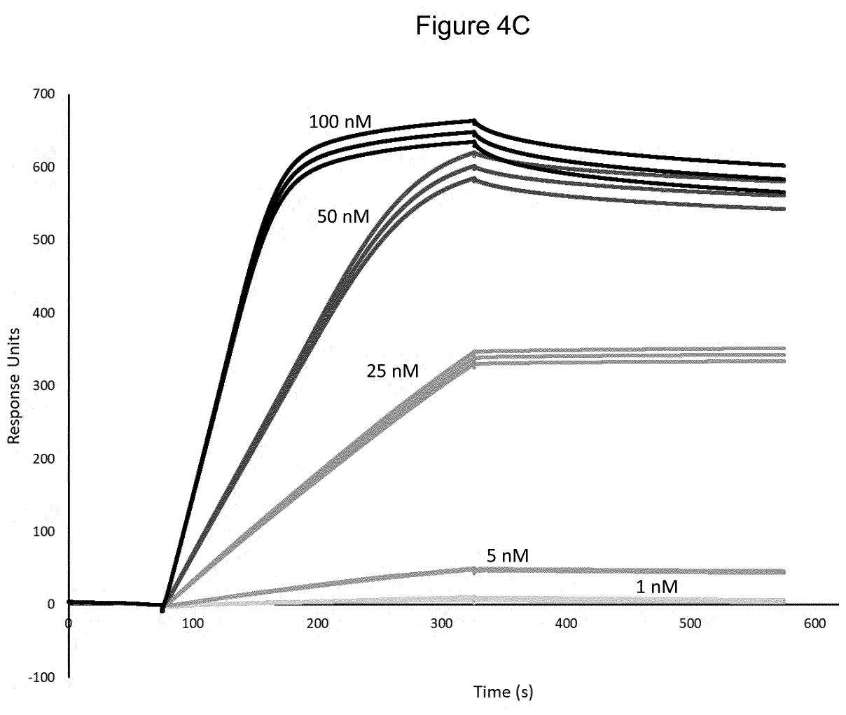

SPR analysis: H₆-ZC69006-ABD was shown to bind to HSA, hCD69 and mCD69 (FIG. 4). From the SPR experiments, the K_{D} values for the respective interactions were estimated to be 52 nM for hCD69, 67 nM for mCD69 and 0.27 nM for HSA.

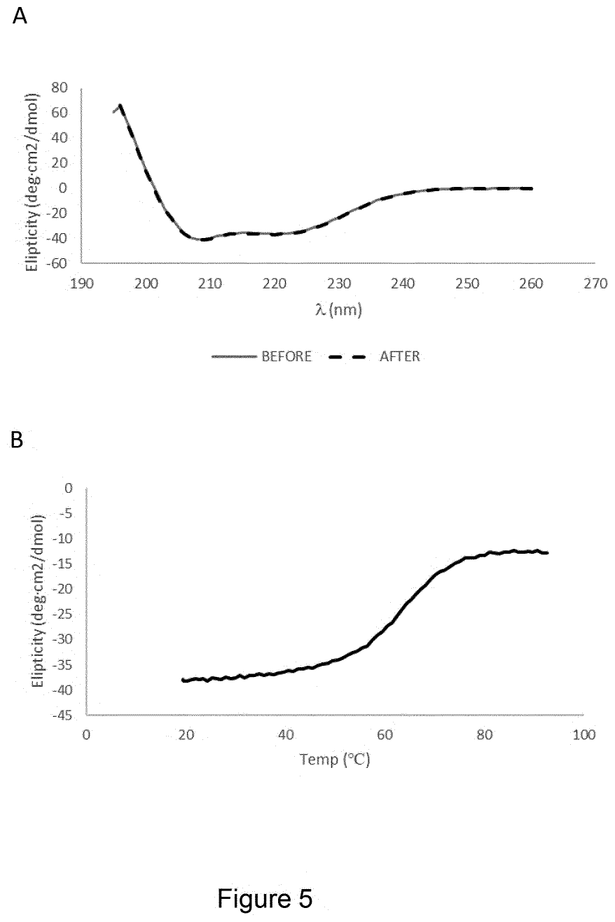

Circular dichroism spectroscopy: The melting temperature for H₆-ZC69006 was determined by circular dichroism spectroscopy and variable temperature measurements to be 70° C. (FIG. 5B). Measuring the CD spectrum before and after heat-induced denaturation demonstrated complete refolding for H₆-ZC69006 (FIG. 5A).

DOTA conjugation: H₆-ZC69006-Cys was successfully conjugated with DOTA to a high degree as determined by MALDI, and subsequently purified using HPLC.

### Example 4

### Characterization of ZC69006 in Cell Assays and In Vivo

### Aims

This Example describes the radiolabeling and evaluation of the DOTA conjugated CD69-binding Z variant ZC69006 prepared as described in Example 3. The radionuclide indium-111 was selected for labeling, because it chelates stably with the DOTA chelator and has a radioactive half-life that is suitable for preclinical evaluation, i.e. one labeled batch can be used for several experiments. Furthermore, indium-111 is a photon emitter suitable for SPECT imaging.

### Methods

Z variant radiolabeling: H₆-ZC69006-Cys was expressed and conjugated to DOTA via the cysteine at the C terminus as described in Example 3. The Z variant ZTAQ, raised against bacterial Taq polymerase and not binding to CD69, was similarly conjugated with DOTA for use as negative control. The polypeptides are denoted DOTA-ZC69006 and DOTA-ZTAQ in the following for brevity. DOTA-ZC69006 and DOTA-ZTAQ were labelled with indium-111 in a buffered solution (pH=5.0-5.5) at elevated temperature. The crude products were purified using solid phase extraction cartridges. Product was eluted with 50% ethanol and formulated in phosphate buffered saline. The radiochemical purity was controlled by HPLC with UV and radio detectors coupled in series. ¹¹¹In-DOTA-ZC69006 and ¹¹¹In-DOTA-ZTAQ were obtained with a radiochemical purity of >95%.

In vitro cell binding: ¹¹¹In-DOTA-ZC69006 was incubated with 0.3-2 million human peripheral blood mononuclear cells (PBMC) or mouse splenic cells, either non-activated or activated by anti-CD3 antibody (1 μg/ml; ab8090, Abcam) in fetal calf serum (FCS). The fraction of CD69+ cells in each preparation was assessed by flow cytometry. Briefly, radioactive ¹¹¹In-DOTA-ZC69006 (target amount 500 kBq, corresponding to 10 nM peptide, incubation volume 1 ml) was added to each cell suspension and incubated for 1 h at 37° C. The cells were then washed and the supernatant collected after centrifugation. The samples and relevant controls (background, references) were measured in a gamma counter (Wizard). All sample measurements were performed in triplicates. Afterwards, the background activity and the remaining activity in the empty Eppendorf vials were measured separately. Cell binding was expressed as % of total incubated amount of ¹¹¹In-DOTA-ZC69006 bound per million cells.

Animal handling: All procedures involving animals were approved by the Animal Ethics Committee of the Swedish Animal Welfare Agency and carried out in accordance with the relevant national and institutional guidelines (“Uppsala university guidelines on animal experimentation”, UFV 2007/724). During each imaging session, the animals (rats and mice) were sedated by gas anesthesia (sevoflurane) through a facemask. Temperature was maintained by warm air supply integrated in the scanner bed. All SPECT/CT examinations were performed in a fully quantified nanoSPECT/CT scanner (Mediso, Hungary). SPECT were acquired using the energy windows suitable for indium-111 emission (energy map: primary peak 245.35 kEv, secondary peak 171.30 kEv), and reconstructed using an iterative algorithm (iterations/subsets 48/3). CT acquisition was performed before all SPECT examinations for anatomical co-registration (semicircular multi-field-of-view; duration 7:46 min; 3 rotations; scan length 231.66 mm; 480 projections; binning 1:4; 50 kV; 600 μA; voxel size 0.25×0.25×0.25 mm).

In vivo imaging in rats: ¹¹¹In-DOTA-ZC69006 distribution in healthy rats was assessed by SPECT/CT imaging and compared to the distribution of ¹¹¹In-DOTA-ZTAQ. Sprague-Dawley rats (n=3, male) were injected in the lateral tail-vein with 2.1-5.3 MBq of ¹¹¹In-DOTA-ZC69006 and examined by SPECT/CT (nanoSPECT, Mediso, Hungary), just after injection. The animal was positioned by a whole-body CT acquisition. Next, a 20 min whole body static SPECT examination was performed. The SPECT/CT examinations were repeated 3 h, 20 h and 48 h post injection. A second group of rats (n=3) was examined similarly, following administration of the negative control molecule ¹¹¹In-DOTA-ZTAQ.

In vivo imaging in mice with islet allograft: NMRI mice (n=5) were transplanted subcutaneously on the left flank with an islet allograft isolated from Balb/c mice. Five or 6 days later, when rejection of the graft was underway, the animals were administered 0.6-0.7 MBq ¹¹¹In-DOTA-ZC69006 in the tail-vein. One mouse was imaged repeatedly over the first 2 h post injection (4 static whole-body scans, 30 min duration each), to determine the optimal imaging time-point. The remaining mice were imaged with a single 30 min static whole-body examination 1 h post injection.

SPECT data analysis and predicted dosimetry in human: SPECT image analysis for all four time-points (0, 3, 20 and 48 h post injection) was performed using the PMOD 3.8 software (PMOD Technologies, Zurich, Switzerland). The aorta, kidney, muscle, liver, bladder and hind leg lymph nodes were segmented using co-registered CT projections as support. The uptake values were converted to standardized uptake values (SUV) by correcting for animal weight and administered amount of ¹¹¹In-DOTA-ZC69006. The biodistribution data over 48 h in healthy rats was used to extrapolate the predicted human dosimetry. Residence times were estimated as described previously and the absorbed doses calculated by the OLINDA/EXM 1.1 software (Vanderbilt, Nashville, USA).

Statistical analysis: Values are given as averages±standard deviations. Statistical analysis and data processing were performed using GraphPad Prism 8.02 for Macintosh/Windows (GraphPad Software Inc) and Microsoft Office Excel 2016 for Macintosh/Windows (Microsoft).

### Results

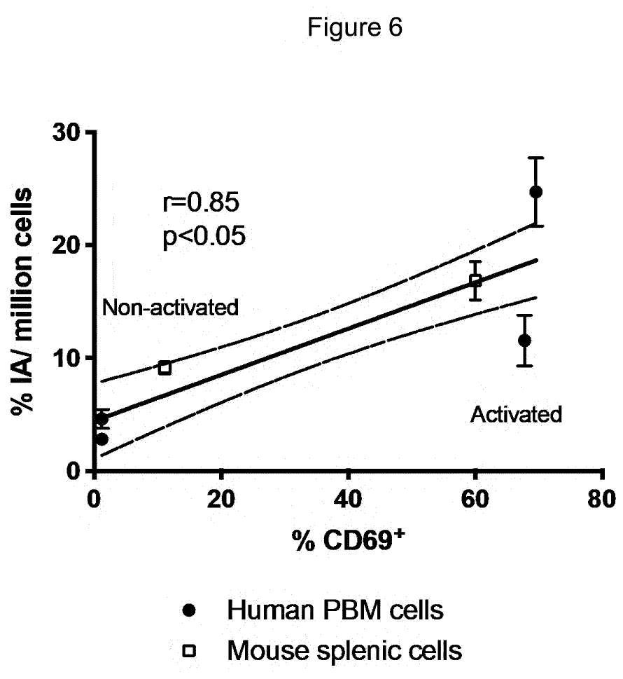

In vitro cell binding: ¹¹¹In-DOTA-ZC69006 binding was higher in CD3 activated human PBMCs compared to resting PBMCs (FIG. 6). A similar increase in binding was seen also in CD3 activated mouse splenic cells (FIG. 6). ¹¹¹In-DOTA-ZC69006 binding correlated well with the fraction of CD69+ cells in all cell preparations (R 2=0.70, p<0.0001).

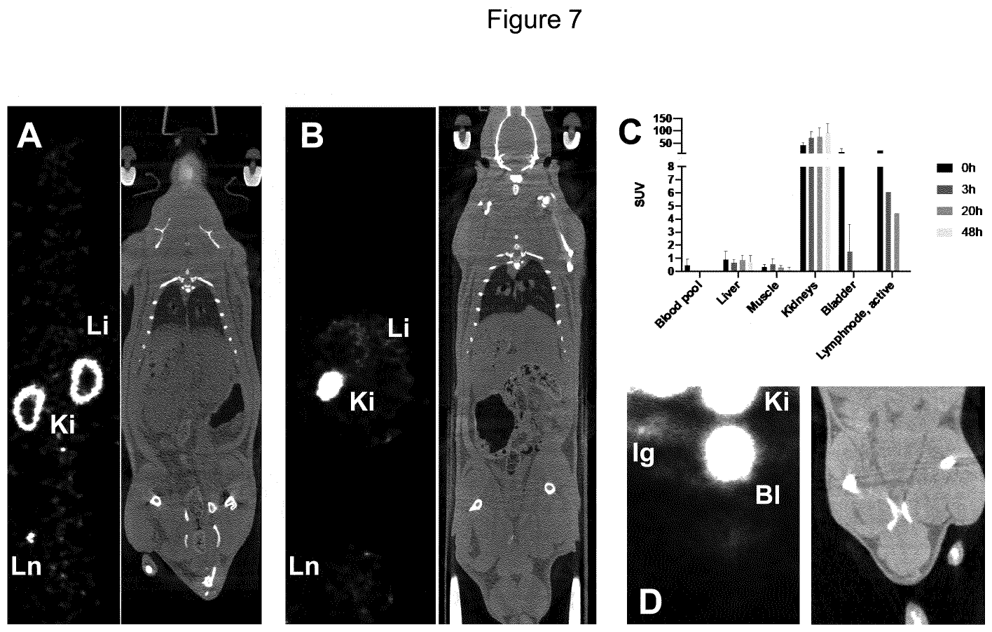

In vivo imaging in rats: ¹¹¹In-DOTA-ZC69006 distributed rapidly throughout the body, with fast clearance from the blood pool combined with renal excretion in all three rats (FIG. 7A). The strong signal in renal cortex was most likely due to re-absorption of ¹¹¹In-DOTA-ZC69006 by the renal tubules, which is a common re-uptake mechanism seen for Z variant peptides when they are used in in vivo imaging. After uptake in the renal tubules, indium-111 is intracellularly trapped, which explains the high retention also seen here up to 48 h post-injection (FIG. 7A).

The uptake of ¹¹¹In-DOTA-ZC69006 in the blood pool was low, as seen in the major arteries and veins, as well as in the left ventricle. This can also be inferred from the very low background binding in the blood rich liver throughout the 48 h imaging time period (FIG. 7A). Spleen and pancreas also exhibited negligible background binding. The pattern for uptake of ¹¹¹In-DOTA-ZC69006 was confirmed by SUV quantification of the SPECT images (FIG. 7C).

Except kidney and bladder, the only tissues that displayed a strong, sustained uptake of ¹¹¹In-DOTA-ZC69006 were small focal uptake sites consistent with the localization of lymph nodes. In a representative rat, shown in FIG. 7A, uptake was seen in the left hind leg lymph node for up to 20 h. Two additional lymph nodes on both contralateral sides of the same animal exhibited uptake throughout the scanning period, but with a lower intensity (not shown). Other lymph nodes did not display any appreciable uptake. Similar patterns, but not in the same lymph nodes, were also seen in the other two rats.

Importantly, the negative control molecule ¹¹¹In-DOTA-ZTAQ did not exhibit any uptake in lymph-nodes in any of the animals (FIG. 7B). Otherwise, ¹¹¹In-DOTA-ZTAQ demonstrated a similar biodistribution as ¹¹¹In-DOTA-ZC69006, including renal excretion.

The predicted absorbed dose of ¹¹¹In-DOTA-ZC69006 in human was the highest in the kidneys (4.65 mGy/MBq) and the whole-body effective dose was 0.16 mSv/MBq.

In vivo imaging in mice with islet allograft: ¹¹¹In-DOTA-ZC69006 displayed rapid renal excretion and low background in all mice, similarly to rats. In a representative mouse examined at day 5 post-transplantation, there was elevated uptake at the site of the allogenic islet graft (ig) (FIG. 7D), but not at the contralateral subcutaneous site lacking a graft. The uptake around the islet graft was clearly visible in all 4 scans over 2 h post-injection in this mouse. Previous experience with this allograft model indicates day 4-5 post transplantation as the time when a strong local immune response should occur in the process of graft rejection.

Two of the mice examined with ¹¹¹In-DOTA-ZC69006 in the morning on day 6 post transplantation also displayed binding at the site of implantation, but of a lower magnitude. This indicated a lower amount of activated immune cells as the rejection process was subsiding. Two additional mice, also transplanted 6 days earlier and scanned at the end of day 6, displayed the lowest binding at the site of the islet allograft, potentially indicating the absence of active immune cells at the site following graft rejection.

### Conclusions

In this Example, DOTA-ZC69006 was radiolabeled with indium-111 to enable optimal preclinical evaluation with regard to long time follow-up of biodistribution. The long half-life allowed repeated in vivo imaging sessions in several animals over the course of a week, in order to follow tracer kinetics. However, indium-111 is a SPECT radionuclide with an undesirable dosimetry profile due to its 3-day radioactive half-life. This was illustrated here by the relatively high radiation dose seen especially in the kidney. The positron-emitting radionuclide gallium-68 in combination with PET would improve sensitivity, resolution, quantification accuracy and reduce radiation dose. Consequently, the further development towards clinical use is contemplated to be focused on ⁶⁸Ga-labelled analogues.

Strong selective and sustained binding was demonstrated in a subcutaneous islet allograft undergoing rejection (FIG. 7D). Importantly, the dynamics of binding of ¹¹¹In-DOTA-ZC69006 captured the expected aspects of graft rejection as mediated by the immune system. Progressively lower uptake at the allograft location was observed over 36 h, from the morning of day 5 to the evening of day 6, potentially indicating the complete rejection of the allograft. These observations are in accordance with previous experience with the same transplantation rejection model.

All three rats examined by SPECT/CT exhibited variable but sustained and clearly detectable uptake of ¹¹¹In-DOTA-ZC69006 in different lymph nodes across the body. This is consistent with the immune response against local, subclinical infection. The specificity of ¹¹¹In-DOTA-ZC69006 for lymph nodes was indirectly demonstrated by performing the same set of experiments using the negative control ¹¹¹In-DOTA-ZTAQ, which has an amino acid sequence that does not confer binding to CD69. No detectable uptake was observed in lymph nodes when using ¹¹¹In-DOTA-ZTAQ. Furthermore, several other Z variants, targeting e.g. HER2 and HER3, have previously been radiolabeled and thoroughly examined in vivo in animals and humans, and focal uptake in lymph nodes has not been observed in such previous studies either. Thus, the lymph node binding of ¹¹¹In-DOTA-ZC69006 is likely to be an active and specific process. These encouraging results warrant the further development of gallium-68 and fluorine-18 labelled analogues of DOTA-ZC69006 for imaging of activated immune cells by PET.

In conclusion, ¹¹¹In-DOTA-ZC69006 is a novel CD69-binding Z variant useful for non-invasive imaging of activated immune cells.

### Example 5

### Affinity Maturation of the First Generation CD69-Binding Z Variant

ZC69006 has an affinity of just over 50 nM to human and murine CD69. For PET tracers, the affinity (K_{D}) is generally inversely correlated to the ability to detect lower number of receptors (Bₘₐₓ). Thus, high affinity is crucial for detecting small changes in CD69-presenting immune cells. In order to achieve a higher affinity, affinity maturation was carried out on ZC69078 identified in Examples 1-2, i.e. the original version of Z variant ZC69006 studied extensively in Examples 3-4.

### Methods

Design of an affinity maturation library: An affinity maturation library was designed with the distribution of varied amino acid positions shown in Table 3 below.

TABLE 3
Design of Z variant library for affinity maturation selection

| Position in the Z | | |
|---|---|---|
| variant sequence | Permitted amino acid residues | Proportions |
| 9 | A, D, E, F, G, I, K, L, M, N, Q, R, S, T, V, W, Y | F: 70%; Rest: 1.9% each |
| 10 | A, D, E, F, G, I, K, L, M, N, Q, R, S, T, V, W, Y | Y: 70%; Rest: 1.9% each |
| 11 | A, D, E, F, G, I, K, L, M, N, Q, R, S, T, V, W, Y | H: 70%; Rest: 1.9% each |
| 13 | A, D, E, F, G, I, K, L, M, N, Q, R, S, T, V, W, Y | M: 70%; Rest: 1.9% each |
| 14 | A, D, E, F, G, I, K, L, M, N, Q, R, S, T, V, W, Y | K: 70%; Rest: 1.9% each |
| 17 | A, D, E, F, G, I, K, L, M, N, Q, R, S, T, V, W, Y | L: 70%; Rest: 1.9% each |
| 18 | A, D, E, F, G, I, K, L, M, N, Q, R, S, T, V, W, Y | K: 70%; Rest: 1.9% each |
| 24 | A, D, E, F, G, I, K, L, M, N, Q, R, S, T, V, W, Y | K: 70%; Rest: 1.9% each |
| 25 | A, D, E, F, G, I, K, L, M, N, Q, R, S, T, V, W, Y | Y: 70%; Rest: 1.9% each |
| 27 | A, D, E, F, G, I, K, L, M, N, Q, R, S, T, V, W, Y | K: 70%; Rest: 1.9% each |
| 28 | A, D, E, F, G, I, K, L, M, N, Q, R, S, T, V, W, Y | E: 70%; Rest: 1.9% each |
| 31 | D, H, I, K, Y | K: 60%; D: 10%; H: 10%; |
| | | I: 10%; Y: 10% |
| 32 | A, D, E, F, G, I, K, L, M, N, Q, R, S, T, V, W, Y | T: 70%; Rest: 1.9% each |
| 35 | A, D, E, F, G, I, K, L, M, N, Q, R, S, T, V, W, Y | K: 70%; Rest: 1.9% each |

DNA encoding the library was obtained from Twist Bioscience. Transformation of the DNA into E. coli BL21(DE3) cells (Merck) gave 6×10⁸ transformants. 96 clones were picked at random and sequenced. The sequencing showed that the library was highly functional and no sequence occurred more than once, except for the original variant ZC69078 (SEQ ID NO:78), which occurred 6 times.

FACS selection: Induced recombinant E. coli cells were washed with 1×PBSP. Cells were resuspended in PBSP containing biotinylated hCD69. The mix was incubated on a rotamixer at RT for 1 h, followed by extended washes with ice-cold PBSP, and resuspended in 150 nM human serum albumin (HSA)-Alexa 647 conjugate and 0.5 μg/ml streptavidin conjugated with R-phycoerythrin (SAPE) (Invitrogen) or neutravidin conjugated with Oregon Green 488 (NAOG) (Life Technologies), followed by incubation on ice for 30 min. The cells were subsequently washed with ice-cold PBSP and resuspended in ice-cold PBSP for sorting in a MoFlo Astrios EQ flow cytometer (Beckman Coulter) or analysis in a Gallios flow cytometer (Beckman Coulter). The E. coli library cells were sorted in a MoFlo Astrios EQ cell sorter (Beckman Coulter).

The sort gate was set to sort out the top fraction of cells displaying Z variants (typically 0.1%) showing the highest R-phycoerythrin or Oregon Green 488 to Alexa Fluor® 647 fluorescence intensity ratio.

Bacteria were sorted into a 1.5 ml tube containing LB medium and chloramphenicol. The sorted cells were incubated for 1 h on rotamixer at 37° C. and thereafter inoculated to 50 ml LB medium with chloramphenicol for overnight cultivation.

### Results and Conclusions

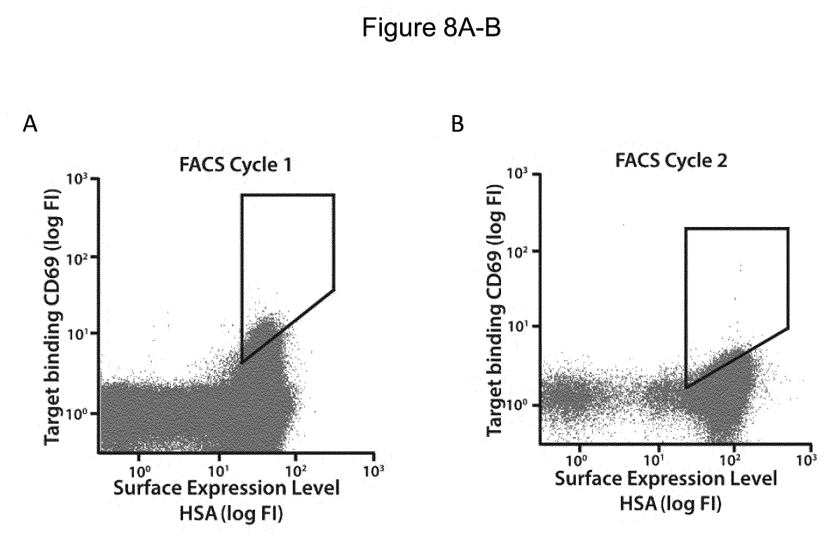

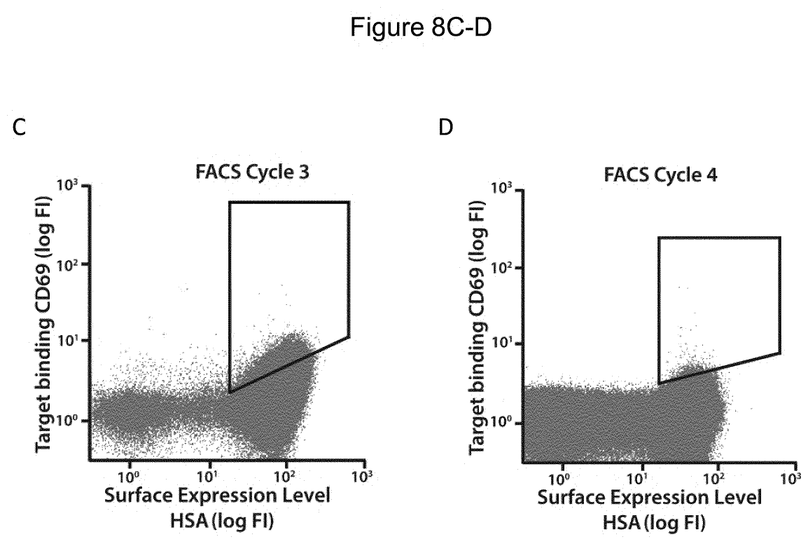

Flow-cytometric sorting for isolation of Z variants with an improved affinity: For isolation of Z variants with an improved affinity for human CD69, the E. coli display library was subjected to four rounds of fluorescence-activated cell sorting (FACS) with alternating rounds of amplification by cell growth. Briefly, cells were incubated with biotinylated hCD69 followed by extensive washes and then incubated with fluorescently labeled streptavidin for subsequent fluorescence-mediated detection of cell-bound hCD69 as well as fluorescently labeled HSA for monitoring of surface expression levels. The incubation of secondary reagents and HSA was performed on ice in order to reduce the dissociation rate of bound hCD69. After an additional washing, the labeled cell library was screened and sorted in a flow cytometer. Selection stringency in terms of target concentration, sorting parameters and sorting gates was increased with each sorting round and typically, the top 0.1% of the library demonstrating the highest ratio of target binding to surface expression, was gated and isolated for amplification and subsequent rounds of sorting. One advantage with cell-based selection systems is the straightforward monitoring of the obtained enrichment throughout the selection process. The visualization of the target-binding properties of the library in the flow cytometer revealed an enrichment of hCD69-positive clones in each sorting round (FIG. 8). After up to four rounds of FACS, isolated cells were spread on semi-solid medium for sequencing and characterization of individual candidates.

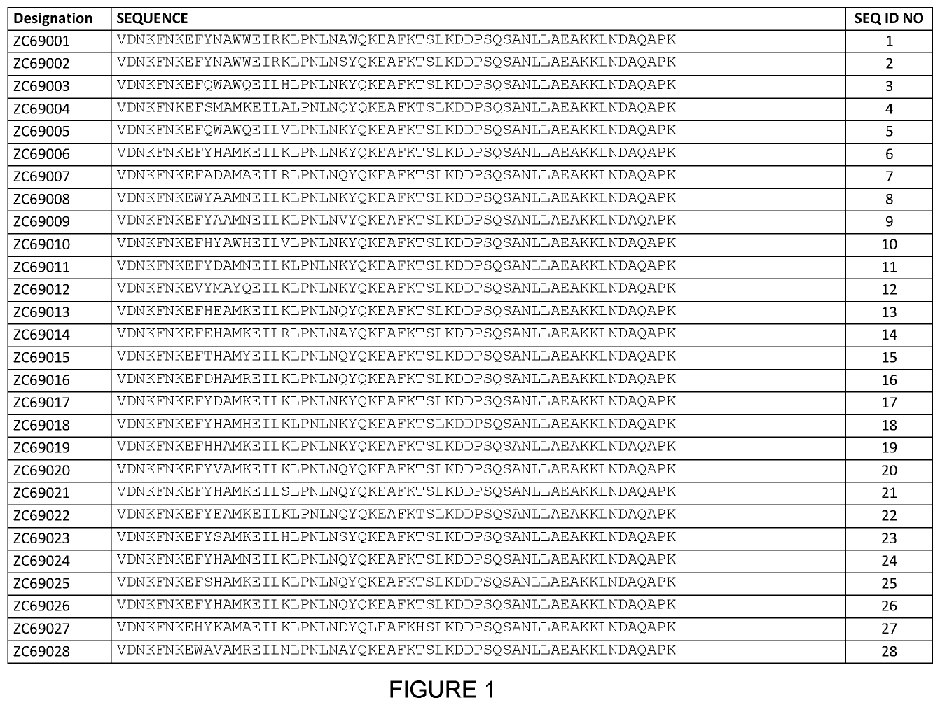

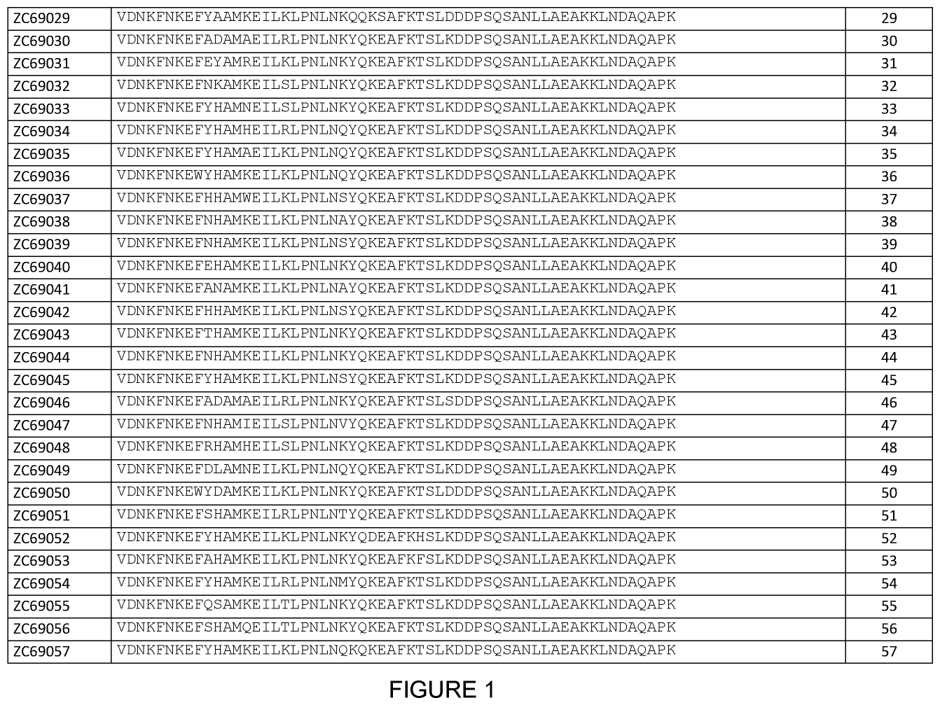

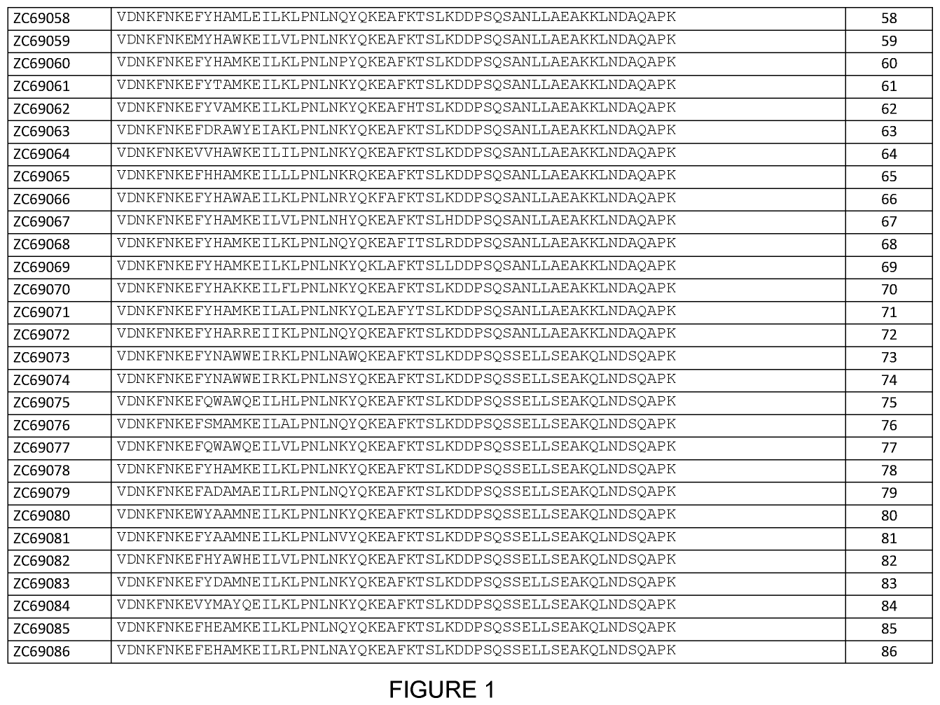

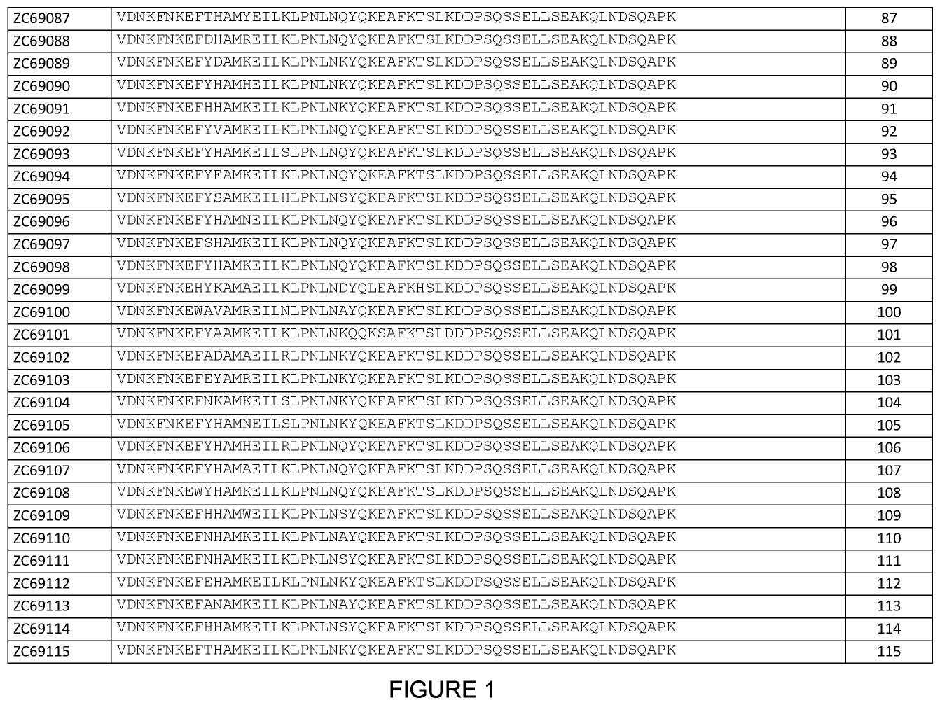

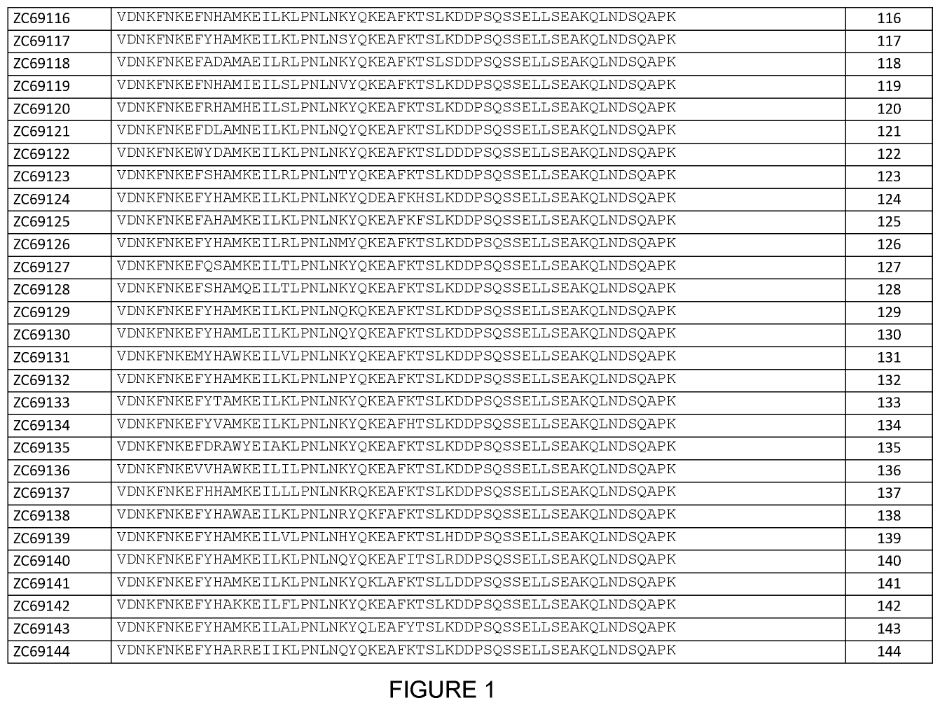

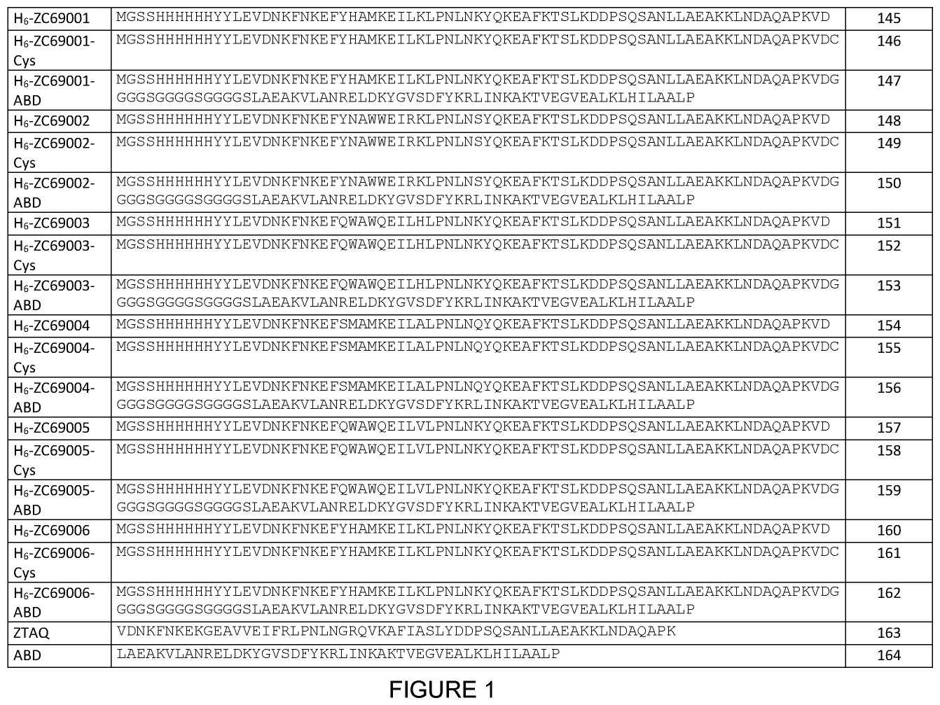

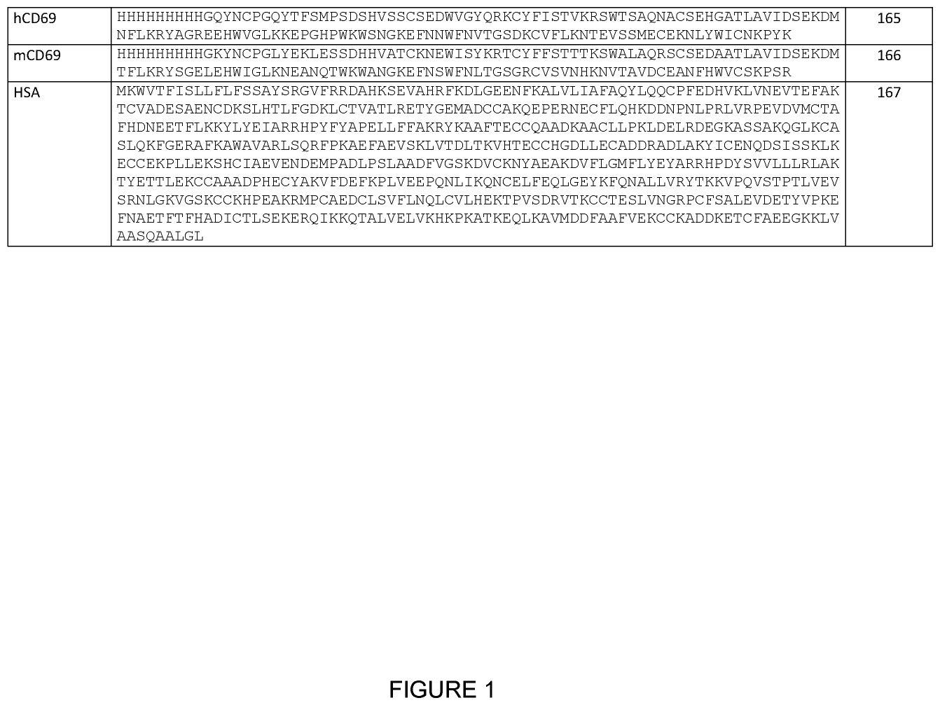

Identification of affinity-matured candidates using FACS: The affinity maturation library was sorted based on surface expression and hCD69-binding. Among the clones isolated from the flow-cytometric sorting, randomly picked variants were sequenced. The amino acid sequences of the 58 amino acid residues long Z variants are listed in FIG. 1 and in the sequence listing as SEQ ID NO:73-77 and 79-144. The deduced CD69-binding motifs extend from residue 8 to residue 36 in each sequence. The 49 amino acid residues long sequences predicted to constitute the complete three-helix bundle within the Z variants extend from residue 7 to residue 55 in each sequence.

Clones appearing more than once among the sequences were selected for further characterization. In FIG. 1 and in the sequence listing, these are denoted SEQ ID NO:73-77.

### Example 6

### Biochemical Characterization of Affinity Matured, CD69-Binding Z Variants

### Methods

Protein production: Z variants from Example 5 that were selected for further characterization were subjected to site-directed mutagenesis in analogy to the creation of ZC69006 from ZC69078 in Example 3, i.e. introduction of the scaffold position mutations S42A, E43N, S46A, Q50K and S54A, creating mutated Z variants named ZC69001 (SEQ ID NO:1), ZC69002 (SEQ ID NO:2), ZC69003 (SEQ ID NO:3), ZC69004 (SEQ ID NO:4) and ZC69005 (SEQ ID NO:5). Genes encoding these five Z variants were each subcloned into tree different E. coli expression vectors based on pET22b (GenScript Biotech Corp) under control of a T7 promoter. Again in analogy to Example 3, all constructs had an N-terminal hexahistidine tag incorporated in the sequence MGSS-H₆-YYLE, and the corresponding gene encoding the Z variant sequence in question followed by the dipeptide -VD. For each Z variant, one of the three constructs had a C-terminal cysteine, and one of the three constructs encoded the H₆-tagged Z variant in fusion with a linker and ABD. The three expression products for each Z variant were denoted H₆-ZC6900 #, H₆-ZC6900 #-Cys and H₆-ZC6900 #-ABD, wherein ZCD6900# corresponds to one of the five Z variants listed above (FIG. 1; SEQ ID NO:145-159). The ligated vector was transformed into E. coli BL21(DE3) cells (Merck) for expression using standard protocols. The recombinant proteins were purified using HisPur Cobalt Resin (#89966, Thermo Scientific) according to the manufacturer's instructions.

SPR evaluation of affinity matured variants: In analogy with Example 3, the target proteins HSA, hCD69 and mCD69 were immobilized on individual CM5 chip surfaces using EDC/NHS coupling chemistry on a Biacore T200 instrument (GE Healthcare), essentially as described in Example 3 for the primary Z variant. The surfaces were inactivated using ethanolamine prior to binding studies. One surface was activated/inactivated for blank subtraction. The five Z variants selected in the affinity maturation procedure described in Example 5 and expressed in the format H₆-ZC6900 #-ABD as described above were injected in the concentration range 1 nM, 5 nM, 25 nM, 50 nM and 100 nM, in triplicates over all four surfaces.

Circular dichroism spectroscopy: Thermal stability and refolding after heat-induced denaturation was measured for each H₆-Z variant using circular dichroism spectroscopy. All measurements were performed on a Chirascan™ instrument (Applied Photophysics Ltd, Surrey, UK). The thermal stability was determined by following the ellipticity at 221 nm during variable temperature measurements (5° C./min from 20° C. to 100° C.). After the heat-induced denaturation, the samples were cooled to RT and left for 15 min before measuring ellipticity at 20° C. from 195 nm to 260 nm in five technical replicates.

### Results

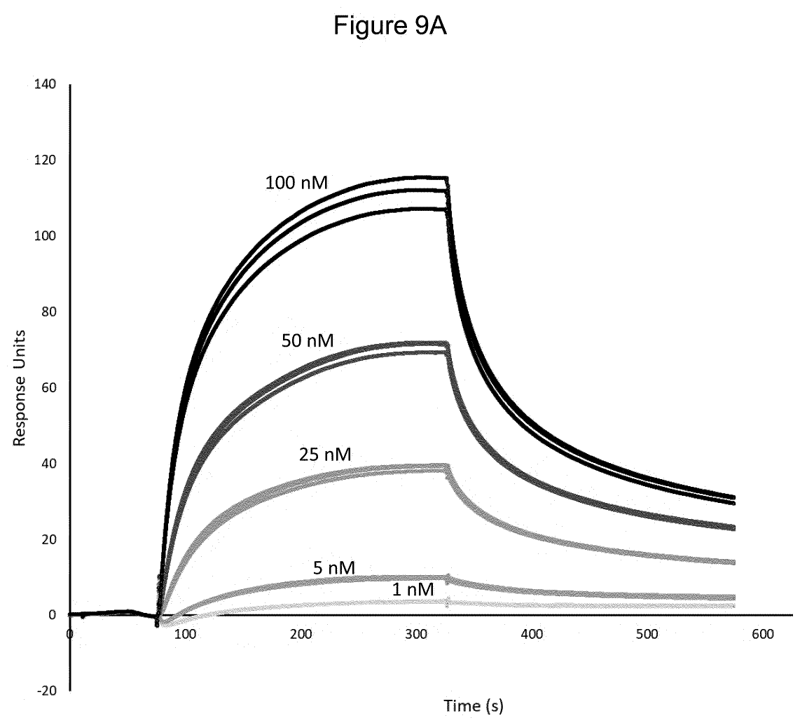

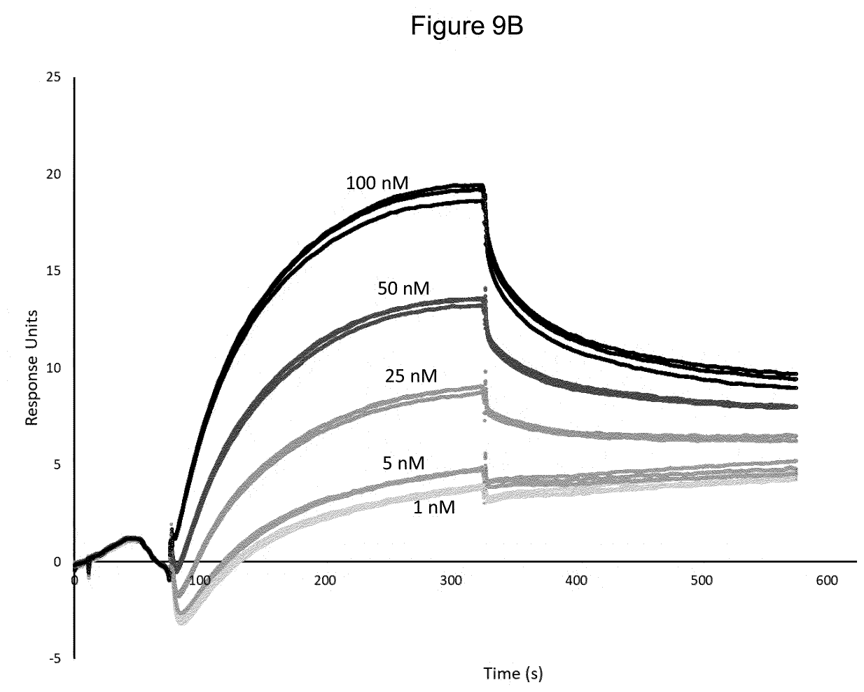

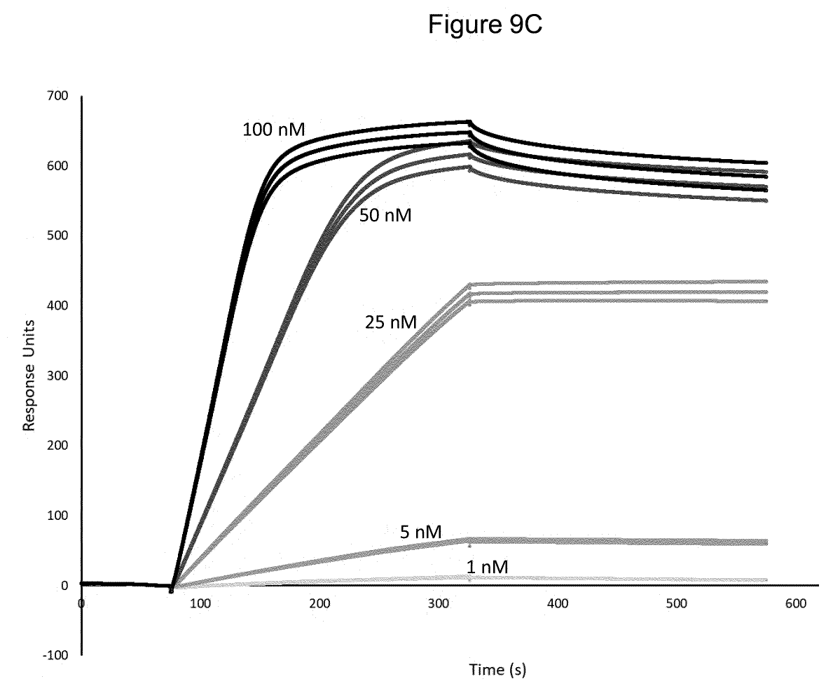

SPR evaluation of affinity matured variants: The affinity towards hCD69 of the five studied Z variants was tested. A representative sensorgram from the SPR analysis is given for Z variant ZC69002 in FIG. 9, and the calculated K_{D} values for interaction human and murine CD69 as well as with with human serum albumin are given in Table 4.

TABLE 4
Affinity constants for tested Z variants from SPR

| Tested Z variant | K_{D} (nM) | | |
|---|---|---|---|
| in the format H₆-Z-(G₄S)₃-ABD | vs. hCD69 | vs. mCD69 | vs. HSA |
| ZC69006 | 52 | 67 | 0.27 |
| ZC69001 | 34 | 34 | 0.17 |
| ZC69002 | 51 | 46 | 0.09 |
| ZC69003 | 49 | ND* | 0.49 |
| ZC69004 | 29 | ND | 0.78 |
| ZC69005 | 30 | ND | 0.52 |

*ND: Not determined

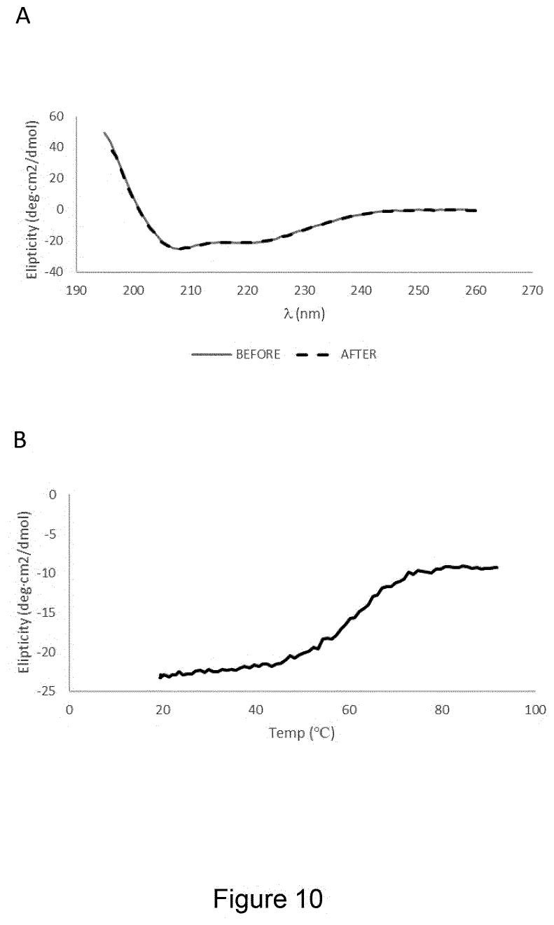

Circular dichroism spectroscopy: The melting temperature for four of the five new Z variants was determined by circular dichroism spectroscopy and variable temperature measurements. As shown in Table 5, they all had similar melting temperatures as ZC69006. Measuring the CD spectrum before and after heat-induced denaturation demonstrated complete refolding for all four variants, as shown for ZC69001 in representative FIG. 10.

TABLE 5
Melting temperatures for tested Z variants from CD

| Tested Z variant | |
|---|---|
| in the format H₆-Z | Tm (° C.) |
| ZC69006 | 62 |
| ZC69001 | 59 |
| ZC69002 | 59 |
| ZC69003 | 62 |
| ZC69005 | 60 |

### Example 7

### Characterization of Affinity Matured CD69-Binding Z Variants in an In Vivo Biodistribution Study

### Aims

The primary Z variant ZC69006 (Examples 1-4) and the five affinity matured Z variants ZC69001, ZC69002, ZC69003, ZC69004 and ZC69005 (Examples 5-6) were studied further.

Outsourced production of large quantities of TCO-conjugated precursor material is expensive. To provide more data for the rational selection of an optimal CD69-binding Z variant, it was decided to produce smaller batches of DOTA-conjugated variants to enable indium-111 radiolabeling and evaluation of critical parameters in vitro and in vivo. This Example describes carrying out this evaluation in order to select an optimal candidate for medical imaging applications.

### Methods

DOTA conjugation: The six tested Z variants were expressed in the format H₆-ZC6900#-Cys and conjugated with DOTA as described for ZC69006 in Example 3. They are referred to as DOTA-ZC6900# for brevity below.

Indium-111 radiolabeling: All reagents were purchased from Sigma-Aldrich (analytical grade or higher) and used without further purification, unless stated otherwise. Indium-111 chloride (370 MBq/ml) was purchased from Curium. Ammonium acetate buffer (0.2 M, pH 5.5) was prepared in plastic flask (200 ml, HDPE low metal resin from Nalgene) by dissolving 0.771 g ammonium acetate in 200 ml water. pH was adjusted by adding a few drops of glacial acetic acid (>99%, TraceSELECT), while measuring with a pH meter (Mettler Toledo). Chelex 100 sodium was added to the buffer, which was allowed to stand in a refrigerator (4° C.) overnight. A NAP-5 column (illustra; GE Healthcare) was pre-treated with 3 ml 1% bovine serum albumin (BSA) and rinsed with excess 6 ml phosphate buffered saline (PBS). Protein LoBinding Eppendorf tubes (1.5 ml) were used for the reaction and elution collection.

Analytical HPLC was performed with a VWR Hitachi Chromaster 5110 pump, a Knauer UV detector 40D, a Bioscan Flow count equipped with an Eckert & Ziegler extended range module 106, a Bioscan B-FC-3300 radioactivity probe, a VWR Hitachi Chromaster A/D Interface box and a Vydac 214MS, 5 μm C4, 50×4.6 mm column. The eluents were: A=0.1% TFA in water, B=0.1% TFA in acetonitrile, with an elution gradient from 5 to 70% B over 15 min ata flow rate of 1.0 ml/min.

For radiolabeling, a hydrochloric acid solution (0.02 M) of ¹¹¹InCl₃ (Curium) was buffered with sodium acetate or HEPES, and pH was adjusted to 5.0-5.5. Thereafter, the respective DOTA-conjugated Z variant (3-14 nmol) dissolved in phosphate buffer was added, with the exception that DOTA conjugation of ZC69004 repeatedly exhibited low yields, so this variant was excluded from the radiolabeling experiment. The reaction mixture (in total 400-500 μl) was incubated at 80° C. for 30-60 min. The crude product was purified on solid phase extraction cartridge (HLB, OASIS; eluted with 1 ml of 50% ethanol) or NAP-5 column (elution with 200 μl PBS×5). See Table 6 for details of the conditions for each variant. The difference in details are due to progressive optimization of the procedure for this class of Z variants. All radiochemical yields (RCYs) were isolated yields and purity was measured by HPLC.

TABLE 6
Radiolabeling conditions

| Z variant | μl ¹¹¹In | MBq | Nmol Z | Reaction | | | | |
|---|---|---|---|---|---|---|---|---|
| as H₆-Z-DOTA | solution | ¹¹¹In | variant | time | | Buffer | pH | Purification |
| ZC69006 | 280 | 14 | 3 | 30 | min | sodium | 5.5 | SPE |
| | | | | | | acetate | | |
| ZC69001 | 150 | 156 | 13.9 | 1 | h | ammonium | 5.2 | NAP-5 |
| | | | | | | acetate | | |
| ZC69002 | 300 | 103 | 10 | 30 | min | HEPES | 5.0 | SPE |
| | | | | | | (0.1M) | | |
| ZC69003 | 120 | 120 | 13.4 | 1 | h | ammonium | 5.5 | NAP-5 |
| | | | | | | acetate | | |
| ZC69005 | 100 | 102 | 4.7 | 30 | min | ammonium | 5.5 | NAP-5 |
| | | | | | | acetate | | |

Assessment of biodistribution by SPECT/CT imaging in rats: All procedures involving animals were approved by the Animal Ethics Committee of the Swedish Animal Welfare Agency and carried out in accordance with the relevant national and institutional guidelines (“Uppsala university guidelines on animal experimentation”, UFV 2007/724).

¹¹¹In-DOTA-Z variant biodistribution in healthy rats was assessed by SPECT/computed tomography (CT) imaging. Sprague-Dawley rats (n=12 in total, n=3 per Z variant, male, weight 310±40 g) were injected in the lateral tail-vein with approximately 8 MBq of indium-111 labeled Z variant (¹¹¹In-DOTA-ZC69006: 3.9±1.6 MBq; ¹¹¹In-DOTA-ZC69001: 12.6±4.6 MBq; ¹¹¹In-DOTA-ZC69002: 5.5±1.3 MBq; ¹¹¹In-DOTA-ZC69003: 8.0±2.6 MBq; ¹¹¹In-DOTA-ZC69005: 9.9±2.6 MBq).

Each animal was examined by SPECT/CT (nanoSPECT, Mediso, Hungary) immediately post injection as well as 3 h, 20 h, 48 h and 72 h post injection. For each scan, the animal was anesthetized and positioned by a whole-body CT acquisition. Next, a 20-minute whole body static SPECT examination was performed. During each imaging session, the rat was sedated by gas anesthesia (sevoflurane) through a facemask. Temperature was maintained by warm air supply integrated in the scanner bed.

All SPECT/CT examinations were performed in a fully quantified nanoSPECT/CT scanner (Mediso, Hungary). SPECT were acquired using the energy windows suitable for indium-111 emission (energy map: primary peak 245.35 kEv, secondary peak 171.30 kEv), and reconstructed using an iterative algorithm (iterations/subsets 48/3). CT acquisition was performed before all SPECT examinations for anatomical co-registration (semicircular multi-field-of-view; duration per bed 7:46 min; 3 rotations; scan length 231.66 mm; 480 projections; binning 1:4; 50 kV; 600 ρA; voxel size 0.25×0.25×0.25 mm).

SPECT image analysis for all time-points was performed using the Nucline software (Mediso, Hungary). The kidneys, liver, heart, lung and muscle were segmented directly on SPECT images using co-registered CT projections as support. Also, distinct uptake in lymph nodes along the main vessels as well as in the hind leg was identified and segmented. The uptake values were decay corrected to the time of administration and converted to standardized uptake values (SUV) by correcting for animal weight and administered amount of ¹¹¹In-DOTA-Z variant.

In animals where distinct uptake in one or more lymph nodes was identified, a post mortem examination was performed. After euthanasia, the lymph node (as well as surrounding tissues, e.g. adipose tissue) was localized and excised. The biopsies were measured for radioactivity in an automated gamma counter (2480 Wizard2™, PerkinElmer). Then, the biopsies were snap frozen, embedded in OCT media, processed into 10 μm sections, placed on SuperFrost object glasses and exposed to a phosphorimager plate with a known reference for quantification (i.e. ex vivo autoradiography).

Statistical analysis: Values are given as averages±standard deviations. Statistical analysis and data processing were performed using GraphPad Prism 8.02 for Macintosh/Windows (GraphPad Software Inc) and Microsoft Office Excel 2016 for Macintosh/Windows (Microsoft).

### Results and Conclusions

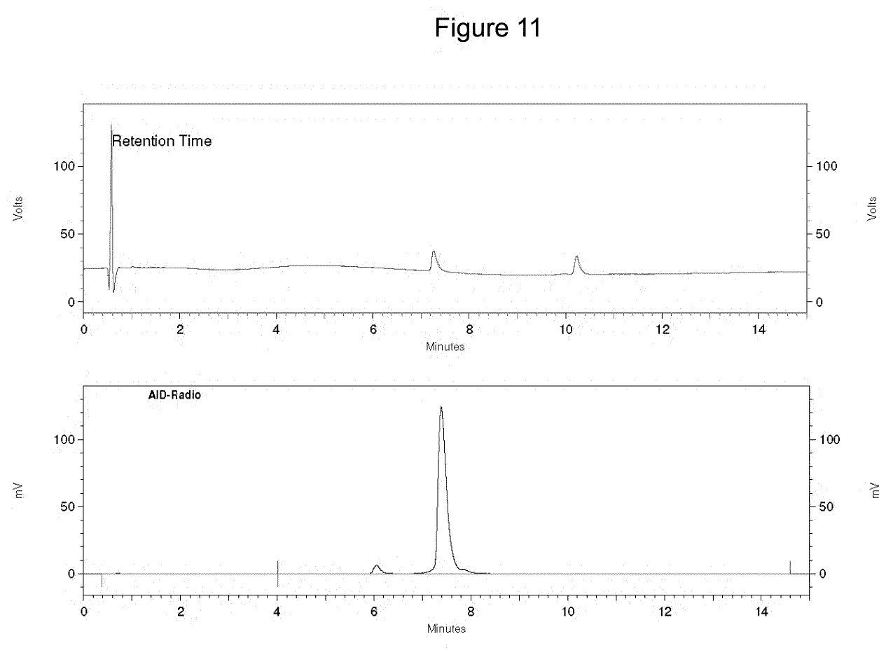

Indium-111 radiolabeling: All Z variants were radiolabeled, except for ZC69004 for which DOTA conjugation repeatedly exhibited low yields. Indium-111 chelation was successful for all other tested variants, i.e. for DOTA-ZC69006, DOTA-ZC69001, DOTA-ZC69002, DOTA-ZC69003 and DOTA-ZC69005, demonstrating acceptable yields and radiochemical purity (Table 7, FIG. 11). The radiolabeled constructs were >80% stable in formulation for up to 4 days.

TABLE 7
Results of radiolabeling tested Z variants with indium-111

| Z variant | | | | | |
|---|---|---|---|---|---|
| as H₆-Z- | | | Aₘ (MBq/ | | |
| DOTA | A (MBq) | Purity | nmol) | RCY | Stability |
| ZC69006 | 14 | 99% | 5 | 5% | Not performed |
| ZC69001 | 85.0 | 99% | 6 | 71% | 91% (4 days) |
| ZC69002 | 92 | 98% | 9 | 90% | 98% (1 day) |
| ZC69003 | 76.5 | 100% | 6 | 84% | 88% (4 days) |
| ZC69005 | 83.2 | 99% | 18 | 93% | 67% (4 days) |

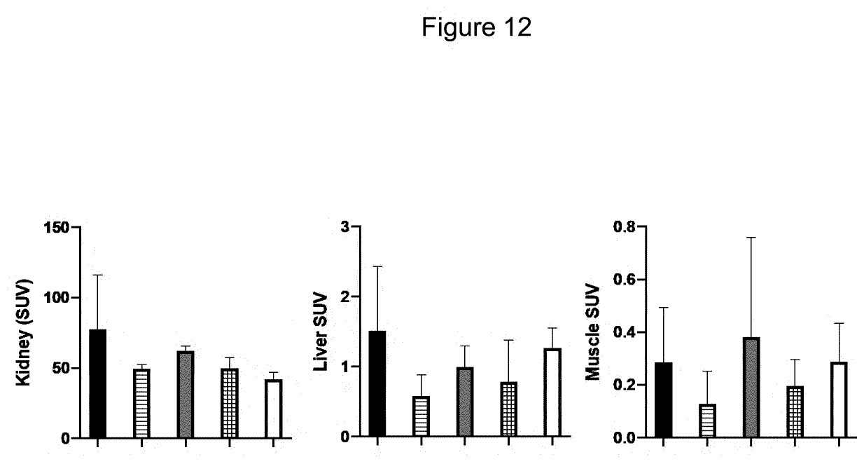

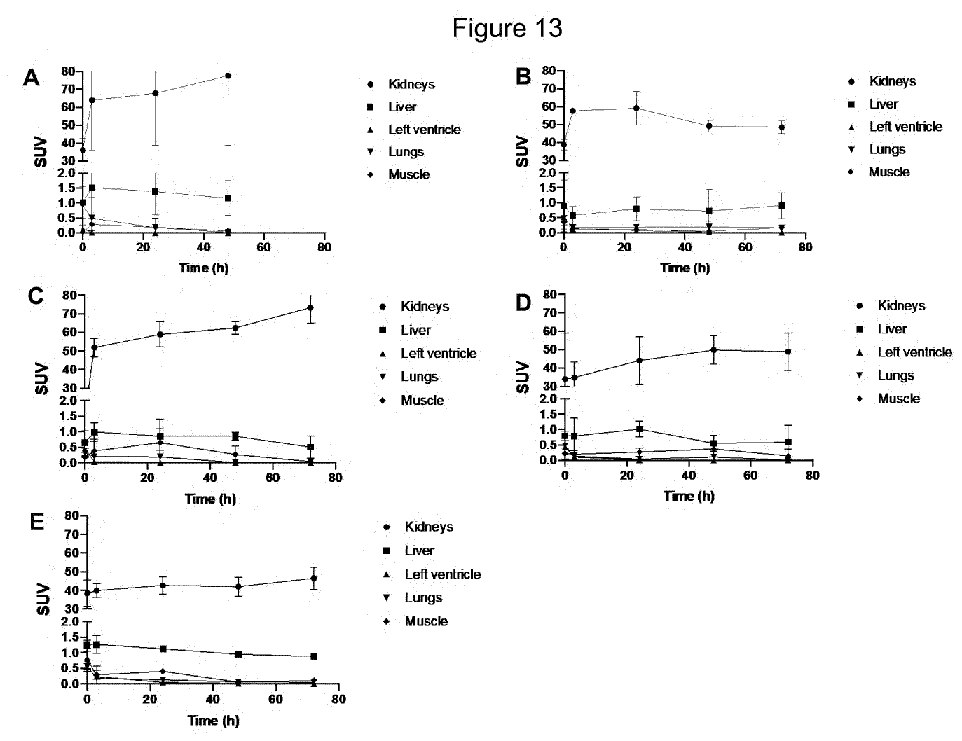

Biodistribution: All indium-111 labeled Z variants exhibited rapid excretion though the kidneys (FIGS. 12 and 13) and washout from most tissues. The uptake and retention in the kidney cortex differed between the Z variants, with ¹¹¹In-DOTA-ZC69006 demonstrating the highest kidney uptake, followed by ¹¹¹In-DOTA-ZC69002. ¹¹¹In-DOTA-ZC69001 and ¹¹¹In-DOTA-ZC69003 had intermediate kidney uptake, while ¹¹¹In-DOTA-ZC69005 exhibited the lowest uptake at the 48 h time point (FIGS. 12 and 13).

For the background binding in liver and muscle tissue, ¹¹¹In-DOTA-ZC69001 demonstrated the lowest binding, followed by ¹¹¹In-DOTA-ZC69003. ¹¹¹In-DOTA-ZC69006, ¹¹¹In-DOTA-ZC69002 and ¹¹¹In-DOTA-ZC69005 all had higher background binding in liver and muscle (FIGS. 12 and 13).

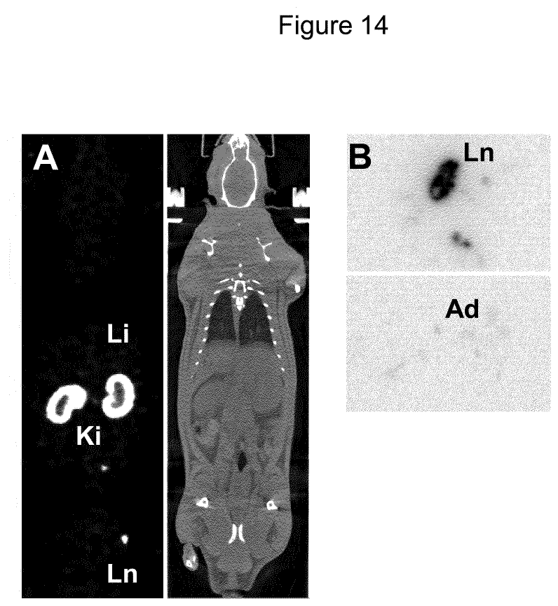

Lymph node targeting: Like the primary Z variant, the affinity matured variants also exhibited strong and sustained uptake in different lymph nodes in the trunk of the body and in the hind body, indicative of an immune response against local, subclinical infection, in some of the animals (FIG. 14A).

The binding as measured by SPECT was confirmed by ex vivo autoradiography, demonstrating a strong signal for the lymph nodes that were positive on the SPECT images, but a negligible signal from the surrounding adipose tissue (FIG. 14B).

### Conclusions and Selection of Candidate

For use in medical imaging in a human, the renal uptake and retention dose should be minimized to reduce the predicted extrapolated absorbed radiation dose to the kidney. Furthermore, the background binding of the radioligand should be minimized for optimal image contrast in lesions or tissues with activated CD69 expressing immune cells. All tested Z variants demonstrated an acceptable kidney dose and background binding. However, based on biodistribution, Z variant ZC69001 seems optimal for further development.

### Example 8

### PET Radiolabeling and Characterization of ZC69001 in an In Vitro Cell Binding Assay

### Aims

This Example describes the radiolabeling and in vitro evaluation of the CD69-binding Z variant ZC69001 in a cell binding assay. The radionuclides gallium-68 and fluorine-18 was selected for labeling in this setting, because both are suitable radionuclides for quantitative PET imaging, further evaluated in Example 9.

### Methods

Gallium-68 radiolabeling of ZC69001: ZC69001-Cys (SEQ ID NO:1 directly followed by a cysteine residue) was produced by chemical peptide synthesis and conjugated with DOTA on the C-terminal cysteine (Almac). DOTA-ZC69001 was labelled with gallium-68 (produced by a generator) in a buffered solution (pH=4.0-5.5) at elevated temperature. The crude products were purified using solid phase extraction cartridges. Product was eluted with 50% ethanol and formulated in phosphate buffered saline. The radiochemical purity was controlled by HPLC with UV and radio detectors coupled in series.

Fluorine-18 radiolabeling of ZC69001: ZC69001-Cys (SEQ ID NO:1 directly followed by a cysteine residue) was obtained functionalized with a trans-cyclooctene (TCO) on the C-terminal cysteine via a PEG3 linker (Almac). This Z variant was produced by chemical synthesis rather than by recombinant expression. ZC69001-TCO was labelled with fluorine-18 by the following three-step procedure: labelling of an activated ester on solid support, conversion to a tetrazine-amide derivative, and conjugation of this tetrazine-amide derivative to ZC69001-TCO. The reaction between the labelled tetrazine and the TCO-modified biomolecule was efficient and rapid, and performed in an aqueous phosphate buffer (pH 7.4) at RT. Purity was assessed by HPLC.

Generation of a CD69 overexpressing cell line: A CHO-K1 cell line was purchased from ATCC and cultured in Ham's F-12 (Biowest), 10% FBS (Sigma) and 1% penicillin/streptomycin (Merck Millipore). The human CD69 cDNA sequence was constructed from the NM_001781.2 NCBI Reference Sequence Database (RefSeq) and purchased from Genscript Biotech Corporation as a pcDNA3.1+/C-(K)DYK vector. The CHO-K1 cells were cultured to 80% confluence before transfection. The CD69 cDNA clone sequence was mixed with Lipofectamine 3000 reagents (Invitrogen) and prepared according to the manufacturer's guideline. Transfected CHO-K1 clones were selected using 1 mg/ml of Geneticin (ThermoFisher) before moving to a pressure concentration of 0.4 mg/ml. Surface expression of CD69 was analyzed by fluorescence activated cell sorting (FACS) using APC conjugated anti-human CD69 antibody (FN50, Biolegend).

Binding studies: Functional binding of ¹⁸F-TZ-ZC69001 was evaluated using the CD69 overexpressing CHO-K1 cell line. Background binding was assessed by blocking the CD69 receptor by pre-incubation with excess of unlabeled ZC69001-Cys. ¹⁸F-TZ-ZC69001 binding was evaluated using viable detached cells incubated with different concentrations of tracers using a gamma counter (Wallac).

### Results and Conclusions

Radiolabeling: Both ⁶⁸Ga-DOTA-ZC69001 and ¹⁸F-TZ-ZC69001 were reproducibly obtained with a radiochemical purity of >95%.

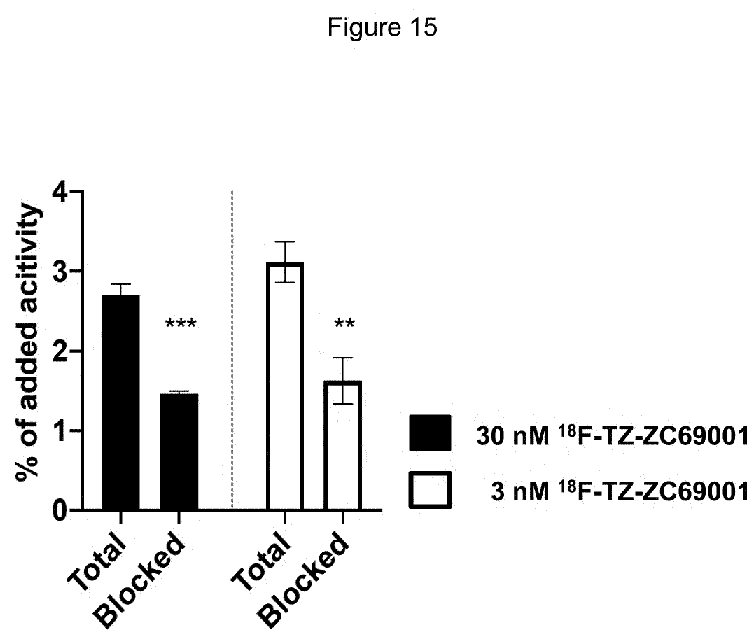

Cell binding studies: ¹⁸F-TZ-ZC69001 bound to CD69 transfected CHO-K1 cells in a manner that could be inhibited by addition of excess ZC69001-Cys (FIG. 15).

### Example 9

### In Vivo Imaging of ZC69001 in Disease Models

### Aims

This Example describes the use of the radiolablelled CD69-binding Z variant ZC69001 for in vivo PET imaging of inflammation and of immunotherapy treated animals. ZC69001 radiolabled with either ⁶⁸Ga or ¹⁸F as described in Example 8 is used.

### Methods

In vivo imaging in induced model of rheumatoid arthritis (RA) in mice: A mouse strain (T-cells knockout, on mixed C57/BL6/NOD background) were administered splenic cells from a transgenic mouse stain (KRN T-cells, C57/BL6 background). After administration, the recipient mice usually develop a model of RA after 2-7 days. The RA is progressive until around 2-3 weeks and associated with increased swelling of the paws as well as infiltration of immune cells. Here, recipient mice (n=8) were given splenic cells from donor mice (n=3). Five of the mice were used for longitudinal PET imaging, while three were used for histological morphometry at different stages of RA progression. The group of five mice were examined by ⁶⁸Ga-DOTA-ZC69001 PET/CT four times—before induction of RA (baseline), and 3, 7 and 12 days after induction of RA. All animals were evaluated for clinical symptoms by weight loss and the “RA score” (sum of grade 0-3 swelling for each of the four paws, total grade from 0 (no symptoms) to 12 (severe RA)).

For PET/CT, a target dose of 2 MBq ⁶⁸Ga-DOTA-ZC69001 was administered in the tail vein. After 1 h, each mouse was anaesthetized and examined by a 30 min whole body PET scan (nanoPET/MRI system, Mediso) and a CT scan for anatomical correlation (nanoSPECT/CT system, Mediso) using a detachable bed.

Binding of ⁶⁸Ga-DOTA-ZC69001 in hind leg joints was evaluated in image analysis software PMOD (PMOD technologies) and expressed as Standardized Uptake Values (SUV) normalized for injected amount of radioactivity and mouse weight to enable inter- and intra-individual comparison.

In vivo imaging of lung inflammation in pigs: Pigs (n=3) were anaesthetized, placed on ventilator and prepared for PET imaging of the lung. Two of the pigs were induced with lung inflammation while one was not. For this study, an established ventilator induced model of lung inflammation (VILI) was used, consisting of repeated lung lavages followed by injurious ventilation. Briefly, repeated lavages were performed with 35 mL/kg warm NaCl 0.9%. The lung function was assessed after each set of three lavages by monitoring ventilator readout and arterial blood gases (p/f ratio, Vd/Vt ratio, oxygen saturation). Lavages were performed until the p/f ratio was recorded as below 100 (severe ARDS) or the maximum of 8 lavages was reached. Then, VILI was started (PEEP=0; Target Ppeak=35; Respiratory Rate (RR)=30; Tidal volume 300 mL, FiO2=100) and continued for appr 1 h.

Each pig was examined by PET over the lungs using a Discovery MI PET/CT (GE healthcare). Two of the pigs, one with induced lung inflammation and one untreated control, were administered 1 MBq/kg ⁶⁸Ga-DOTA-ZC69001 intravenously and examined by dynamic PET/CT for 90 min and a whole body static scan. The second pig with induced lung inflammation was administered 1 MBq/kg ⁶⁸Ga-DOTA-ZAM106, a non-CD69-binding control peptide of the same molecular size and scaffold.

Chimeric antigen receptor T cells (CAR-T) immunotherapy mouse model: ¹⁸F-TZ-ZC69001, prepared and tested as described in Example 8, is evaluated in a mouse model of CAR-T cell treatment of lymphoma. Murine B-cell lymphoma cells are injected subcutaneously. When tumors form, the mice are treated with murine CD20-directed CAR-T cells secreting neutrophil-activating protein (NAP) upon target cell recognition. Five to seven days later, activated T cells in the tumor area are imaged with ¹⁸F-TZ-ZC69001. Mice are treated with conventional CD20-directed CAR-T cells or mock-transduced T cells as control. 10 MBq ¹⁸F-TZ-ZC69001 is administered in the tail vein under sevoflurane anesthesia. Animals are examined by dynamic PET for up to 2 h using a nanoPET/MRI scanner (Mediso). After the PET examination, the animals are euthanized, organs excised, weighed and measured in a gamma counter. Part of the tumor and reference organs are snap frozen, embedded in OCT media, sectioned and exposed to a phosphorimager plate to generate ex vivo autoradiography images of ¹⁸F-TZ-ZC69001 distribution in the tumor microenvironment. Part of the tumor is fixed in PFA and stained with e.g. H&E, and immunostained for CD69 and other relevant immune and tumor markers.

### Results

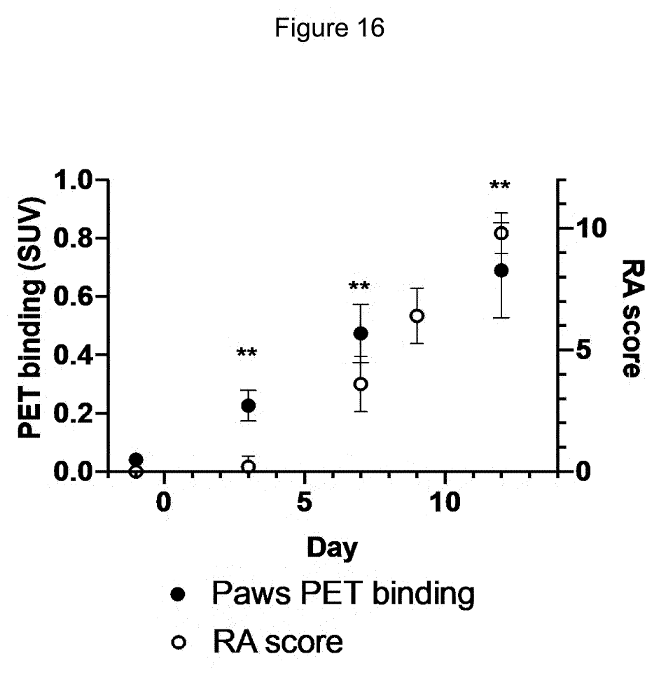

In vivo imaging in induced model of rheumatoid arthritis (RA) in mice: ⁶⁸Ga-DOTA-ZC69001 binding was low or negligible in all mice at baseline, before induction of RA. PET binding was elevated already on day 3, increasing further on day 7 and finally on day 12 (FIG. 16). Importantly, the elevated PET binding preceded the increase in RA score and clinical symptoms with several days, indicating that ⁶⁸Ga-DOTA-ZC69001 binding in joints is an early biomarker for sub-clinical RA by binding to infiltrating CD69 positive immune cells.

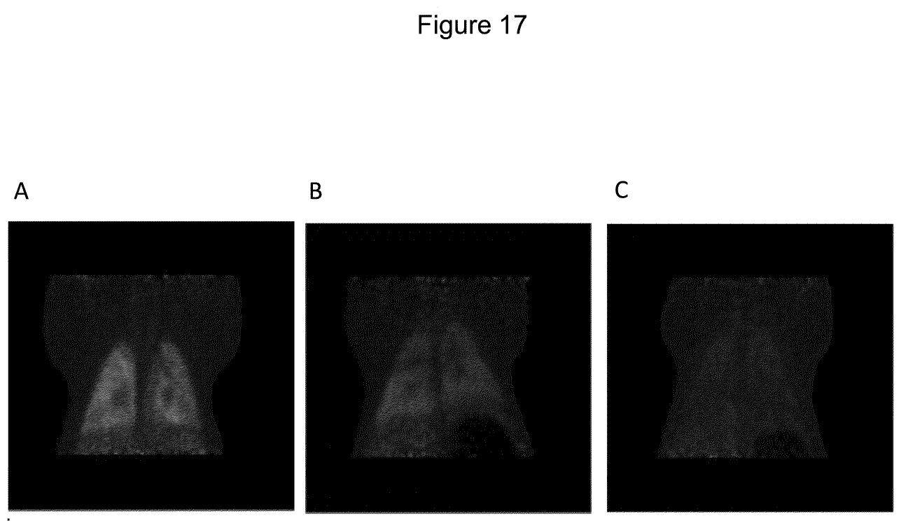

In vivo imaging of lung inflammation in pigs: ⁶⁸Ga-DOTA-ZC69001 demonstrated elevated binding of ⁶⁸Ga-DOTA-ZC69001 in the lung of pig with induced lung injury and inflammation, compared to lung binding in a control pig. In contrast, the non-CD69-binding control peptide 88 Ga-DOTA-ZAM106 exhibited negligible binding in a pig with lung inflammation, indicating that the scaffold peptide itself does not accumulate in inflamed tissue in a non-specific manner (FIG. 17).

Chimeric antigen receptor T cells (CAR-T) immunotherapy mouse model: ¹⁸F-TZ-ZC69001 is expected to accumulate in the tumors of immunotherapy treated animals, but not in the controls. The PET results are expected to be confirmed by ex vivo autoradiography and correlative staining.

### Itemized List of Embodiments

1. CD69-binding polypeptide, comprising a CD69-binding motif BM, which motif consists of an amino acid sequence selected from:

i)
(SEQ ID NO: 168)
EX₂X₃X₄AX₆X₇EIX₁₀ X₁₁LPNLX₁₆
X₁₇X₁₈QK X₂₁AFKX₂₅X₂₆LKD

wherein, independently of each other,
X₂ is selected from F, H, V and W;
X₃ is selected from A, D, E, H, N, Q, S, T, Y;
X₄ is selected from A, D, E, H, K, M, N, S, V, W and Y;
X₆ is selected from M, W and Y;
X₇ is selected from A, H, K, N, Q, R, W and Y;
X₁₀ is selected from L and R;
X₁₁ is selected from A, H, K, R, S and V;
X₁₆ is selected from N and T;
X₁₇ is selected from A, D, K, Q, S and V;
X₁₈ is selected from W and Y;
X₂₁ is selected from E and S;
X₂₅ is selected from H and T; and
X₂₆ is selected from K and S;
   and
   ii) an amino acid sequence which has at least 93% identity to the sequence defined in i).

2. CD69-binding polypeptide according to item 1, wherein, in sequence i),
X₂ is F;
X₃ is selected from Q, S and Y;
X₄ is selected from H, M, N and W;
X₆ is selected from M and W;
X₇ is selected from K, Q and W;
X₁₀ is selected from L and R;
X₁₁ is selected from A, H, K and V;
X₁₆ is selected from N and T;
X₁₇ is selected from A, K, Q and S;
X₁₈ is selected from W and Y;
X₂₁ is E;
X₂₅ is T; and
X₂₆ is selected from K and S.

3. CD69-binding polypeptide according to item 1 or 2, wherein sequence i) fulfills at least five of the ten conditions I-X:
I. X₂ is F;
II. X₃ is Y;
III. X₄ is H, Nor W;
IV. X₆ is M or W;
V. X₁₀ is L;
VI. X₁₁ is K;
VII X₁₇ is K or Q;
VIII. X₁₈ is Y;
IX. X₂₁ is E; and
X. X₂₅ is T.

4. CD69-binding polypeptide according to item 3, wherein sequence i) fulfills at least six of the ten conditions I-X.

5. CD69-binding polypeptide according to item 4, wherein sequence i) fulfills at least seven of the ten conditions I-X.

6. CD69-binding polypeptide according to item 5, wherein sequence i) fulfills at least eight of the ten conditions I-X.

7. CD69-binding polypeptide according to item 6, wherein sequence i) fulfills at least nine of the ten conditions I-X.

8. CD69-binding polypeptide according to item 7, wherein sequence i) fulfills all of the ten conditions I-X.

9. CD69-binding polypeptide according to any one of items 1-8, wherein X₃ is Y, X₄ is H and X₁₁ is K.

10. CD69-binding polypeptide according to any one of items 1-9, wherein X₃ is Y, X₄ is H and X₂₁ is E.

11. CD69-binding polypeptide according to any one of items 1-10, wherein X₃ is Y, X₁₁ is K and X₁₈ is Y.

12. CD69-binding polypeptide according to any one of items 1-11, wherein X₁₁ is K, X₁₈ is Y and X₂₁ is E.

13. CD69-binding polypeptide according to any preceding item, wherein sequence i) corresponds to the amino acid sequence from position 8 to position 36 in a sequence selected from the group consisting of SEQ ID NO:1-72.

14. CD69-binding polypeptide according to item 13, wherein sequence i) corresponds to the amino acid sequence from position 8 to position 36 in a sequence selected from the group consisting of SEQ ID NO:1-29.

15. CD69-binding polypeptide according to item 14, wherein sequence i) corresponds to the amino acid sequence from position 8 to position 36 in a sequence selected from the group consisting of SEQ ID NO:1-28.

16. CD69-binding polypeptide according to item 15, wherein sequence i) corresponds to the amino acid sequence from position 8 to position 36 in a sequence selected from the group consisting of SEQ ID NO:1-26.

17. CD69-binding polypeptide according to item 16, wherein sequence i) corresponds to the amino acid sequence from position 8 to position 36 in a sequence selected from the group consisting of SEQ ID NO:1-6.

18. CD69-binding polypeptide according to item 17, wherein sequence i) corresponds to the amino acid sequence from position 8 to position 36 in a sequence selected from the group consisting of SEQ ID NO:1-2 and 6.

19. CD69-binding polypeptide according to item 18, wherein sequence i) corresponds to the amino acid sequence from position 8 to position 36 in a sequence selected from the group consisting of SEQ ID NO:1-2.

20. CD69-binding polypeptide according to item 19, wherein sequence i) corresponds to the amino acid sequence from position 8 to position 36 in SEQ ID NO:1.

21. CD69-binding polypeptide according to any preceding item, wherein said CD69-binding motif (BM) forms part of a three-helix bundle protein domain.

22. CD69-binding polypeptide according to item 21, wherein said CD69-binding motif (BM) essentially constitutes two alpha helices with an interconnecting loop, within said three-helix bundle protein domain.

23. CD69-binding polypeptide according to item 22, wherein said three-helix bundle protein domain is selected from domains of bacterial receptor proteins.

24. CD69-binding polypeptide according to item 23, wherein said three-helix bundle protein domain is selected from the five different three-helical domains of Protein A from Staphylococcus aureus, such as domain B, and derivatives thereof.

25. CD69-binding polypeptide according to item 24, wherein said three-helix bundle protein domain is a variant of protein Z, which is derived from domain B of staphylococcal Protein A.

26. CD69-binding polypeptide according to any preceding item, which comprises a binding module (BMod), the amino acid sequence of which is selected from:

iii)
K-[BM]-DPSQSXₐX_{b}LLX_{c} EAKX_{d}LXₑX_{f}X_{g}Q;

wherein
[BM] is a CD69-binding motif as defined in any one of items 1-12;
Xₐ is selected from A and S;
X_{b} is selected from E and N;
X_{c} is selected from A, S and C;
X_{d} is selected from K and Q;
Xₑ is selected from E, N and S;
X_{f} is selected from D, E and S; and
X_{g} is selected from A and S; and
   iv) an amino acid sequence which has at least 93% identity to a sequence defined in iii).

27. CD69-binding polypeptide according to item 26, wherein sequence iii) corresponds to the amino acid sequence from position 7 to position 55 in a sequence selected from the group consisting of SEQ ID NO:1-72.

28. CD69-binding polypeptide according to item 27, wherein sequence iii) corresponds to the amino acid sequence from position 7 to position 55 in a sequence selected from the group consisting of SEQ ID NO:1-29.

29. CD69-binding polypeptide according to item 28, wherein sequence iii) corresponds to the amino acid sequence from position 7 to position 55 in a sequence selected from the group consisting of SEQ ID NO:1-28.

30. CD69-binding polypeptide according to item 29, wherein sequence iii) corresponds to the amino acid sequence from position 7 to position 55 in a sequence selected from the group consisting of SEQ ID NO:1-26.

31. CD69-binding polypeptide according to item 30, wherein sequence iii) corresponds to the amino acid sequence from position 7 to position 55 in a sequence selected from the group consisting of SEQ ID NO:1-6.

32. CD69-binding polypeptide according to item 31, wherein sequence iii) corresponds to the amino acid sequence from position 7 to position 55 in a sequence selected from the group consisting of SEQ ID NO:1-2 and 6.

33. CD69-binding polypeptide according to item 32, wherein sequence iii) corresponds to the amino acid sequence from position 7 to position 55 in a sequence selected from the group consisting of SEQ ID NO:1-2.

34. CD69-binding polypeptide according to item 33, wherein sequence iii) corresponds to the amino acid sequence from position 7 to position 55 in SEQ ID NO:1.

35. CD69-binding polypeptide according to item 26, wherein sequence iii) corresponds to the amino acid sequence from position 7 to position 55 in a sequence selected from the group consisting of SEQ ID NO:73-144.

36. CD69-binding polypeptide according to item 35, wherein sequence iii) corresponds to the amino acid sequence from position 7 to position 55 in a sequence selected from the group consisting of SEQ ID NO:73-101.

37. CD69-binding polypeptide according to item 36, wherein sequence iii) corresponds to the amino acid sequence from position 7 to position 55 in a sequence selected from the group consisting of SEQ ID NO:73-100.

38. CD69-binding polypeptide according to item 37, wherein sequence iii) corresponds to the amino acid sequence from position 7 to position 55 in a sequence selected from the group consisting of SEQ ID NO:73-98.

39. CD69-binding polypeptide according to item 38, wherein sequence iii) corresponds to the amino acid sequence from position 7 to position 55 in a sequence selected from the group consisting of SEQ ID NO:73-78.

40. CD69-binding polypeptide according to item 39, wherein sequence iii) corresponds to the amino acid sequence from position 7 to position 55 in a sequence selected from the group consisting of SEQ ID NO:73-74 and 78.

41. CD69-binding polypeptide according to item 40, wherein sequence iii) corresponds to the amino acid sequence from position 7 to position 55 in a sequence selected from the group consisting of SEQ ID NO:73-74.

42. CD69-binding polypeptide according to item 41, wherein sequence iii) corresponds to the amino acid sequence from position 7 to position 55 in SEQ ID NO:73.

43. CD69-binding polypeptide according to any preceding item, which comprises an amino acid sequence selected from:

v)
FN-[BMod]-AP;

wherein [BMod] is a CD69-binding module as defined in any one of items 26-42; and
vi) an amino acid sequence which has at least 90% identity to a sequence defined in v).

44. CD69-binding polypeptide according to any one of items 1-42, which comprises an amino acid sequence selected from:

vii)
YA-[BMod]-AP;

wherein [BMod] is a CD69-binding module as defined in any one of items 26-42; and
viii) an amino acid sequence which has at least 90% identity to a sequence defined in vii).

45. CD69-binding polypeptide according to any preceding item, wherein the CD69-binding motif forms part of a polypeptide comprising an amino acid sequence selected from

ADNNFNK-[BM]-DPSQSANLLSEAKKLNESQAPK;
ADNKFNK-[BM]-DPSQSANLLAEAKKLNDAQAPK;
ADNKFNK-[BM]-DPSVSKEILAEAKKLNDAQAPK;
ADAQQNNFNK-[BM]-DPSQSTNVLGEAKKLNESQAPK;
AQHDE-[BM]-DPSQSANVLGEAQKLNDSQAPK;
VDNKFNK-[BM]-DPSQSANLLAEAKKLNDAQAPK;
VDNKFNK-[BM]-DPSQSSELLSEAKQLNDSQAPK;
VDNKFNK-[BM]-DPSQSSELLSEAKKLNDSQAPK
AEAKYAK-[BM]-DPSESSELLSEAKKLNKSQAPK;
VDAKYAK-[BM]-DPSQSSELLAEAKKLNDAQAPK;
VDAKYAK-[BM]-DPSQSSELLAEAKKLNDSQAPK;
AEAKYAK-[BM]-DPSQSSELLSEAKKLNDSQAPK;
AEAKYAK-[BM]-DPSQSSELLSEAKKLNDSQAP;
AEAKFAK-[BM]-DPSQSSELLSEAKKLNDSQAPK;
AEAKFAK-[BM]-DPSQSSELLSEAKKLNDSQAP;
AEAKYAK-[BM]-DPSQSSELLAEAKKLNDAQAPK;
AEAKYAK-[BM]-DPSQSSELLSEAKKLSESQAPK;
AEAKYAK-[BM]-DPSQSSELLSEAKKLSESQAP;
AEAKFAK-[BM]-DPSQSSELLSEAKKLSESQAPK;
AEAKFAK-[BM]-DPSQSSELLSEAKKLSESQAP;
AEAKYAK-[BM]-DPSQSSELLAEAKKLSEAQAPK;
AEAKYAK-[BM]-QPEQSSELLSEAKKLSESQAPK;
AEAKYAK-[BM]-DPSQSSELLSEAKKLESSQAPK;
AEAKYAK-[BM]-DPSQSSELLSEAKKLESSQAP;
AEAKYAK-[BM]-DPSQSSELLAEAKKLESAQAPK;
AEAKYAK-[BM]-QPEQSSELLSEAKKLESSQAPK;
AEAKYAK-[BM]-DPSQSSELLSEAKKLSDSQAPK;
AEAKYAK-[BM]-DPSQSSELLSEAKKLSDSQAP;
AEAKYAK-[BM]-DPSQSSELLAEAKKLSDSQAPK;
AEAKYAK-[BM]-DPSQSSELLAEAKKLSDAQAPK;
AEAKYAK-[BM]-QPEQSSELLSEAKKLSDSQAPK;
VDAKYAK-[BM]-DPSQSSELLSEAKKLNDSQAPK;
VDAKYAK-[BM]-DPSQSSELLAEAKKLNDAQAPK;
VDAKYAK-[BM]-DPSQSSELLSEAKKLSESQAPK;
VDAKYAK-[BM]-DPSQSSELLAEAKKLSEAQAPK;
VDAKYAK-[BM]-QPEQSSELLSEAKKLSESQAPK;
VDAKYAK-[BM]-DPSQSSELLSEAKKLESSQAPK;
VDAKYAK-[BM]-DPSQSSELLAEAKKLESAQAPK;
VDAKYAK-[BM]-QPEQSSELLSEAKKLESSQAPK;
VDAKYAK-[BM]-DPSQSSELLSEAKKLSDSQAPK;
VDAKYAK-[BM]-DPSQSSELLAEAKKLSDSQAPK;
VDAKYAK-[BM]-DPSQSSELLAEAKKLSDAQAPK;
VDAKYAK-[BM]-QPEQSSELLSEAKKLSDSQAPK;
VDAKYAK-[BM]-DPSQSSELLAEAKKLNKAQAPK;
AEAKYAK-[BM]-DPSQSSELLAEAKKLNKAQAPK;
and
ADAKYAK-[BM]-DPSQSSELLSEAKKLNDSQAPK;

wherein [BM] is a CD69-binding motif as defined in any one of items 1-20.

46. CD69-binding polypeptide according to any one of items 1-44, which comprises an amino acid sequence selected from:

xvii)
VDNKFNK-[BM]-DPSQSSELLSEAKQLNDSQAPK;

wherein [BM] is a CD69-binding motif as defined in any one of items 1-20; and
xviii) an amino acid sequence which has at least 89% identity to the sequence defined in xvii).

47. CD69-binding polypeptide according to item 46, wherein sequence xvii) is selected from the group consisting of SEQ ID NO:73-144.

48. CD69-binding polypeptide according to item 47, wherein sequence xvii) is selected from the group consisting of SEQ ID NO:73-101.

49. CD69-binding polypeptide according to item 48, wherein sequence xvii) is selected from the group consisting of SEQ ID NO:73-100.

50. CD69-binding polypeptide according to item 49, wherein sequence xvii) is selected from the group consisting of SEQ ID NO:73-98.

51. CD69-binding polypeptide according to item 50, wherein sequence xvii) is selected from the group consisting of SEQ ID NO:73-78.

52. CD69-binding polypeptide according to item 51, wherein sequence xvii) is selected from the group consisting of SEQ ID NO:73-74 and 78.

53. CD69-binding polypeptide according to item 52, wherein sequence xvii) is selected from the group consisting of SEQ ID NO:73-74.

54. CD69-binding polypeptide according to item 53, wherein sequence xvii) is SEQ ID NO:73.

55. CD69-binding polypeptide according to any one of items 1-44, which comprises an amino acid sequence selected from:

xix)
VDNKFNK-[BM]-DPSQSANLLAEAKKLNDAQAPK;

wherein [BM] is a CD69-binding motif as defined in any one of items 1-20; and
xx) an amino acid sequence which has at least 89% identity to the sequence defined in xix).

56. CD69-binding polypeptide according to item 55, wherein sequence xix) is selected from the group consisting of SEQ ID NO:1-72.

57. CD69-binding polypeptide according to item 56, wherein sequence xix) is selected from the group consisting of SEQ ID NO:1-29.

58. CD69-binding polypeptide according to item 57, wherein sequence xix) is selected from the group consisting of SEQ ID NO:1-28.

59. CD69-binding polypeptide according to item 58, wherein sequence xix) is selected from the group consisting of SEQ ID NO:1-26.

60. CD69-binding polypeptide according to item 59, wherein sequence xix) is selected from the group consisting of SEQ ID NO:1-6.

61. CD69-binding polypeptide according to item 60, wherein sequence xix) is selected from the group consisting of SEQ ID NO:1-2 and 6.

62. CD69-binding polypeptide according to item 61, wherein sequence xix) is selected from the group consisting of SEQ ID NO:1-2.

63. CD69-binding polypeptide according to item 62, wherein sequence xix) is SEQ ID NO:1.

64. CD69-binding polypeptide according to any one of items 1-44, which comprises an amino acid sequence selected from:

xxi)
VDNKFNK-[BM]-DPSQSSELLSEAKKLNDSQAPK;

wherein [BM] is a CD69-binding motif as defined in any one of items 1-20; and
xxii) an amino acid sequence which has at least 89% identity to the sequence defined in xxi).

65. CD69-binding polypeptide according to any one of items 1-44, which comprises an amino acid sequence selected from:

xxiii)
AEAKYAK-[BM]-DPSQSSELLSEAKKLNDSQAPK;

wherein [BM] is a CD69-binding motif as defined in any one of items 1-20; and
xxiv) an amino acid sequence which has at least 89% identity to the sequence defined in xxiii).

66. CD69-binding polypeptide according to any preceding item, which is capable of binding to CD69 such that the K_{D} value of the interaction with CD69 is at most 1×10⁻⁶ M, such as at most 5×10⁻⁷ M, such as at most 1×10⁻⁷ M, such as at most 5×10⁻⁸ M, such as at most 1×10⁻⁸ M, such as at most 5×10⁻⁸ M.

67. CD69-binding polypeptide according to any preceding item, which comprises additional amino acids at the C terminus and/or N terminus.

68. CD69-binding polypeptide according to item 67, wherein said additional amino acid(s) improve(s) production, purification, stabilization in vivo or in vitro, coupling or detection of the polypeptide.

69. CD69-binding polypeptide according to any preceding item in a multimeric form comprising at least two CD69-binding polypeptide monomer units, the amino acid sequences of which may be the same or different.

70. CD69-binding polypeptide according to item 69, wherein said monomer units are covalently coupled together.

71. CD69-binding polypeptide according to item 69, wherein said CD69-binding polypeptide monomer units are expressed as a fusion protein.

72. CD69-binding polypeptide according to any one of items 69-71 in dimeric form.

73. Fusion protein or conjugate, comprising
a first moiety consisting of a CD69-binding polypeptide according to any preceding item; and
a second moiety consisting of a polypeptide having a desired biological activity.

74. Fusion protein or conjugate according to item 73, wherein said desired biological activity is a therapeutic activity.

75. Fusion protein or conjugate according to item 73, wherein said desired biological activity is a binding activity.

76. Fusion protein or conjugate according to item 73, wherein said desired biological activity is an enzymatic activity.

77. Fusion protein or conjugate according to item 75, wherein said binding activity is albumin binding activity which increases the in vivo half-life of the fusion protein or conjugate.

78. Fusion protein or conjugate according to any one of items 73-77 further comprising at least one linker.

79. CD69-binding polypeptide, fusion protein or conjugate according to any preceding item, further comprising a label.

80. CD69-binding polypeptide, fusion protein or conjugate according to item 79, wherein said label is selected from the group consisting of fluorescent dyes and metals, chromophoric dyes, chemiluminescent compounds, bioluminescent proteins, enzymes, radionuclides, radioactive particles and pretargeting recognition tags.

81. CD69-binding polypeptide, fusion protein or conjugate according to any one of items 79-80, for use in labeling or targeting cells and tissues which have a high expression of CD69.

82. CD69-binding polypeptide, fusion protein or conjugate according to any one of items 79-80 which is labeled, directly or indirectly, with an imaging agent, such as a radioactive agent.

83. Polynucleotide encoding a CD69-binding polypeptide or fusion protein according to any one of items 1-78.

84. Expression vector comprising the polynucleotide according to item 83.

85. Host cell comprising the expression vector according to item 84.

86. Method of producing the CD69-binding polypeptide, fusion protein or complex according to any one of items 1-78, comprising
culturing the host cell according to item 85 under conditions permissive of expression of said polypeptide from its expression vector; and
isolating the polypeptide.

87. Composition comprising a CD69-binding polypeptide, fusion protein or conjugate according to any one of items 1-82 and at least one pharmaceutically acceptable excipient or carrier.

88. Composition according to item 87, further comprising at least one additional active agent, such as an immune response modifying agent.

89. CD69-binding polypeptide, fusion protein or conjugate according to any one of items 1-82 or a composition according to item 87 or 88 for use as a medicament, as a diagnostic agent in vivo and/or as a prognostic agent in vivo.

90. CD69-binding polypeptide, fusion protein, conjugate or composition for use according to item 89 as a medicament in the treatment of a CD69-related disorder.

91. CD69-binding polypeptide, fusion protein, conjugate or composition for use according to item 89 as a diagnostic agent in the in vivo diagnosis of a CD69-related disorder.

92. CD69-binding polypeptide, fusion protein, conjugate or composition for use according to item 89 as a prognostic agent in the in vivo prognosis of a CD69-related disorder.

93. CD69-binding polypeptide, fusion protein, conjugate or composition for use according to any one of items 90-92, wherein said CD69-related disorder is an autoinflammatory disease.

94. CD69-binding polypeptide, fusion protein, conjugate or composition for use according to item 93, wherein said autoinflammatory disease is type 1 diabetes (T1D).

95. Method of treatment of a CD69-related disorder, comprising administering to a subject in need thereof an effective amount of a CD69-binding polypeptide, fusion protein or conjugate according to any one of items 1-82 or a composition according to item 87 or 88.

96. Method of detecting CD69, comprising providing a sample suspected to contain CD69, contacting said sample with a CD69-binding polypeptide, fusion protein or conjugate according to any one of items 1-82 or a composition according to item 87 or 88, and detecting the binding of the CD69-binding polypeptide, fusion protein, conjugate or composition to indicate the presence of CD69 in the sample.

97. Diagnostic or prognostic method for determining the presence of CD69 in a subject comprising the steps of:
a) contacting the subject, or a sample isolated from the subject, with a CD69-binding polypeptide, fusion protein or conjugate according to any one of items 1-82 or a composition according to item 87 or 88, and
b) obtaining a value corresponding to the amount of the CD69-binding polypeptide, fusion protein, conjugate or composition that has bound in said subject or to said sample.

98. The diagnostic or prognostic method according to item 97, which is a method for diagnosis in vivo or prognosis in vivo, for example via medical imaging, in which said contacting in step a) consists of contacting said subject with said polypeptide, fusion protein, conjugate or composition.

99. The diagnostic or prognostic method according to item 97, which is an in vitro method and in which said contacting in step a) consists of contacting a sample previously isolated from said subject with said polypeptide, fusion protein, conjugate or composition.

1. A CD69-binding polypeptide, comprising a CD69-binding motif BM, which motif consists of an amino acid sequence selected from:
i)
(SEQ ID NO: 168)
EX₂X₃X₄AX₆X₇EIX₁₀ X₁₁LPNLX₁₆
X₁₇X₁₈QK X₂₁AFKX₂₅X₂₆LKD
wherein, independently of each other,
X₂ is selected from F, H, V and W;
X₃ is selected from A, D, E, H, N, Q, S, T, Y;
X₄ is selected from A, D, E, H, K, M, N, S, V, W and Y;
X₅ is selected from M, W and Y;
X₇ is selected from A, H, K, N, Q, R, W and Y;
X₁₀ is selected from L and R;
X₁₁ is selected from A, H, K, R, S and V;
X₁₆ is selected from N and T;
X₁₇ is selected from A, D, K, Q, S and V;
X₁₈ is selected from W and Y;
X₂₁ is selected from E and S;
X₂₅ is selected from H and T; and
X₂₆ is selected from K and S;
and
ii) an amino acid sequence which has at least 93% identity to the sequence defined in i).

2. The CD69-binding polypeptide according to claim 1, wherein, in sequence i),
X₂ is F;
X₃ is selected from Q, S and Y;
X₄ is selected from H, M, N and W;
X₅ is selected from M and W;
X₇ is selected from K, Q and W;
X₁₀ is selected from L and R;
X₁₁ is selected from A, H, K and V;
X₁₆ is selected from N and T;
X₁₇ is selected from A, K, Q and S;
X₁₈ is selected from W and Y;
X₂₁ is E;
X₂₅ is T; and
X₂₆ is selected from K and S.

3. The CD69-binding polypeptide according to claim 1, wherein sequence i) fulfills at least five of the ten conditions I-X:
I. X₂ is F;
II. X₃ is Y;
III. X₄ is H, N or W;
IV. X₆ is M or W;
V. X₁₀ is L;
VI. X₁₁ is K;
VII X₁₇ is K or 0;
VIII. X₁₈ is Y;
IX. X₂₁ is E; and
X. X₂₅ is T.

4. The CD69-binding polypeptide according to claim 1, wherein sequence i) corresponds to the amino acid sequence from position 8 to position 36 in a sequence selected from the group consisting of SEQ ID NO:1-72, SEQ ID NO:1-29, SEQ ID NO:1-28, SEQ ID NO:1-26, SEQ ID NO:1-6, SEQ ID NO:1-2 and 6, SEQ ID NO:1-2, and SEQ ID NO:1.

5. The CD69-binding polypeptide according to claim 1, wherein said CD69-binding motif (BM) forms part of a three-helix bundle protein domain, said three-helix bundle protein domain for example being a variant of protein Z, which is derived from domain B of staphylococcal Protein A.

6. The CD69-binding polypeptide according to claim 1, which comprises a binding module (BMod), the amino acid sequence of which is selected from:
iii)
K-[BM]-DPSQSXₐX_{b}LLX_{c} EAKX_{d}LXₑX_{f}X_{g}Q;
wherein
[BM] is a CD69-binding motif as defined in claim 1;
Xₐ is selected from A and S;
X_{b} is selected from E and N;
X_{c} is selected from A, S and C;
X_{d} is selected from K and Q;
Xₑ is selected from E, N and S;
X_{f} is selected from D, E and S; and
X_{g} is selected from A and S; and
iv) an amino acid sequence which has at least 93% identity to a sequence defined in iii).

7. The CD69-binding polypeptide according to claim 6, wherein sequence iii) corresponds to the amino acid sequence from position 7 to position 55 in a sequence selected from
the group consisting of SEQ ID NO:1-72, SEQ ID NO:1-29, SEQ ID NO:1-28, SEQ ID NO:1-26, SEQ ID NO:1-6, SEQ ID NO:1-2 and 6, SEQ ID NO:1-2, SEQ ID NO:1 or
the group consisting of SEQ ID NO:73-144, SEQ ID NO:73-101 SEQ ID NO:73-100, SEQ ID NO:73-98, SEQ ID NO:73-78, SEQ ID NO:73-74 and 78, SEQ ID NO:73-74, SEQ ID NO:73.

8. The CD69-binding polypeptide according to claim 1, which is capable of binding to CD69 such that the K_{D} value of the interaction with CD69 is at most 1×10⁻⁶ M, 5×10⁻⁷ M, 1×10⁻⁷ M, 5×10⁻⁸ M, 1×10⁻⁸ M, or 5×10⁻⁸ M.

9. A Fusion protein or conjugate, comprising
a first moiety consisting of a CD69-binding polypeptide according to claim 1; and
a second moiety consisting of a polypeptide having a desired biological activity.

10. The CD69-binding polypeptide according to claim 1, further comprising a label selected from the group consisting of fluorescent dyes and metals, chromophoric dyes, chemiluminescent compounds, bioluminescent proteins, enzymes, radionuclides, radioactive particles and pretargeting recognition tags.

11. The CD69-binding polypeptide according to claim 10, for use in labeling or targeting cells and tissues which have a high expression of CD69.

12. The CD69-binding polypeptide according to claim 10 which is labeled, directly or indirectly, with an imaging agent, or a radioactive agent.

13. A Polynucleotide encoding a CD69-binding polypeptide or fusion protein according to claim 1.

14. The Composition comprising a CD69-binding polypeptide according to claim 1 and at least one pharmaceutically acceptable excipient or carrier.

15. The CD69-binding polypeptide according to claim 1 for use as a medicament, as a diagnostic agent in vivo or as a prognostic agent in vivo.

16. The CD69-binding polypeptide according to claim 15 for use as a diagnostic agent in the in vivo diagnosis of a CD69-related disorder.


SEQUENCE LISTING (190 sequences)

SEQ ID NO: 1 - protein, 58 aa, Artificial sequence
  Engineered CD69-binding polypeptide
VDNKFNKEFYNAWWEIRKLPNLNAWQKEAFKTSLKDDPSQSANLLAEAKKLNDAQAPK

SEQ ID NO: 2 - protein, 58 aa, Artificial sequence
  Engineered CD69-binding polypeptide
VDNKFNKEFYNAWWEIRKLPNLNSYQKEAFKTSLKDDPSQSANLLAEAKKLNDAQAPK

SEQ ID NO: 3 - protein, 58 aa, Artificial sequence
  Engineered CD69-binding polypeptide
VDNKFNKEFQWAWQEILHLPNLNKYQKEAFKTSLKDDPSQSANLLAEAKKLNDAQAPK

SEQ ID NO: 4 - protein, 58 aa, Artificial sequence
  Engineered CD69-binding polypeptide
VDNKFNKEFSMAMKEILALPNLNQYQKEAFKTSLKDDPSQSANLLAEAKKLNDAQAPK

SEQ ID NO: 5 - protein, 58 aa, Artificial sequence
  Engineered CD69-binding polypeptide
VDNKFNKEFQWAWQEILVLPNLNKYQKEAFKTSLKDDPSQSANLLAEAKKLNDAQAPK

SEQ ID NO: 6 - protein, 58 aa, Artificial sequence
  Engineered CD69-binding polypeptide
VDNKFNKEFYHAMKEILKLPNLNKYQKEAFKTSLKDDPSQSANLLAEAKKLNDAQAPK

SEQ ID NO: 7 - protein, 58 aa, Artificial sequence
  Engineered CD69-binding polypeptide
VDNKFNKEFADAMAEILRLPNLNQYQKEAFKTSLKDDPSQSANLLAEAKKLNDAQAPK

SEQ ID NO: 8 - protein, 58 aa, Artificial sequence
  Engineered CD69-binding polypeptide
VDNKFNKEWYAAMNEILKLPNLNKYQKEAFKTSLKDDPSQSANLLAEAKKLNDAQAPK

SEQ ID NO: 9 - protein, 58 aa, Artificial sequence
  Engineered CD69-binding polypeptide
VDNKFNKEFYAAMNEILKLPNLNVYQKEAFKTSLKDDPSQSANLLAEAKKLNDAQAPK

SEQ ID NO: 10 - protein, 58 aa, Artificial sequence
  Engineered CD69-binding polypeptide
VDNKFNKEFHYAWHEILVLPNLNKYQKEAFKTSLKDDPSQSANLLAEAKKLNDAQAPK

SEQ ID NO: 11 - protein, 58 aa, Artificial sequence
  Engineered CD69-binding polypeptide
VDNKFNKEFYDAMNEILKLPNLNKYQKEAFKTSLKDDPSQSANLLAEAKKLNDAQAPK

SEQ ID NO: 12 - protein, 58 aa, Artificial sequence
  Engineered CD69-binding polypeptide
VDNKFNKEVYMAYQEILKLPNLNKYQKEAFKTSLKDDPSQSANLLAEAKKLNDAQAPK

SEQ ID NO: 13 - protein, 58 aa, Artificial sequence
  Engineered CD69-binding polypeptide
VDNKFNKEFHEAMKEILKLPNLNQYQKEAFKTSLKDDPSQSANLLAEAKKLNDAQAPK

SEQ ID NO: 14 - protein, 58 aa, Artificial sequence
  Engineered CD69-binding polypeptide
VDNKFNKEFEHAMKEILRLPNLNAYQKEAFKTSLKDDPSQSANLLAEAKKLNDAQAPK

SEQ ID NO: 15 - protein, 58 aa, Artificial sequence
  Engineered CD69-binding polypeptide
VDNKFNKEFTHAMYEILKLPNLNQYQKEAFKTSLKDDPSQSANLLAEAKKLNDAQAPK

SEQ ID NO: 16 - protein, 58 aa, Artificial sequence
  Engineered CD69-binding polypeptide
VDNKFNKEFDHAMREILKLPNLNQYQKEAFKTSLKDDPSQSANLLAEAKKLNDAQAPK

SEQ ID NO: 17 - protein, 58 aa, Artificial sequence
  Engineered CD69-binding polypeptide
VDNKFNKEFYDAMKEILKLPNLNKYQKEAFKTSLKDDPSQSANLLAEAKKLNDAQAPK

SEQ ID NO: 18 - protein, 58 aa, Artificial sequence
  Engineered CD69-binding polypeptide
VDNKFNKEFYHAMHEILKLPNLNKYQKEAFKTSLKDDPSQSANLLAEAKKLNDAQAPK

SEQ ID NO: 19 - protein, 58 aa, Artificial sequence
  Engineered CD69-binding polypeptide
VDNKFNKEFHHAMKEILKLPNLNKYQKEAFKTSLKDDPSQSANLLAEAKKLNDAQAPK

SEQ ID NO: 20 - protein, 58 aa, Artificial sequence
  Engineered CD69-binding polypeptide
VDNKFNKEFYVAMKEILKLPNLNQYQKEAFKTSLKDDPSQSANLLAEAKKLNDAQAPK

SEQ ID NO: 21 - protein, 58 aa, Artificial sequence
  Engineered CD69-binding polypeptide
VDNKFNKEFYHAMKEILSLPNLNQYQKEAFKTSLKDDPSQSANLLAEAKKLNDAQAPK

SEQ ID NO: 22 - protein, 58 aa, Artificial sequence
  Engineered CD69-binding polypeptide
VDNKFNKEFYEAMKEILKLPNLNQYQKEAFKTSLKDDPSQSANLLAEAKKLNDAQAPK

SEQ ID NO: 23 - protein, 58 aa, Artificial sequence
  Engineered CD69-binding polypeptide
VDNKFNKEFYSAMKEILHLPNLNSYQKEAFKTSLKDDPSQSANLLAEAKKLNDAQAPK

SEQ ID NO: 24 - protein, 58 aa, Artificial sequence
  Engineered CD69-binding polypeptide
VDNKFNKEFYHAMNEILKLPNLNQYQKEAFKTSLKDDPSQSANLLAEAKKLNDAQAPK

SEQ ID NO: 25 - protein, 58 aa, Artificial sequence
  Engineered CD69-binding polypeptide
VDNKFNKEFSHAMKEILKLPNLNQYQKEAFKTSLKDDPSQSANLLAEAKKLNDAQAPK

SEQ ID NO: 26 - protein, 58 aa, Artificial sequence
  Engineered CD69-binding polypeptide
VDNKFNKEFYHAMKEILKLPNLNQYQKEAFKTSLKDDPSQSANLLAEAKKLNDAQAPK

SEQ ID NO: 27 - protein, 58 aa, Artificial sequence
  Engineered CD69-binding polypeptide
VDNKFNKEHYKAMAEILKLPNLNDYQLEAFKHSLKDDPSQSANLLAEAKKLNDAQAPK

SEQ ID NO: 28 - protein, 58 aa, Artificial sequence
  Engineered CD69-binding polypeptide
VDNKFNKEWAVAMREILNLPNLNAYQKEAFKTSLKDDPSQSANLLAEAKKLNDAQAPK

SEQ ID NO: 29 - protein, 58 aa, Artificial sequence
  Engineered CD69-binding polypeptide
VDNKFNKEFYAAMKEILKLPNLNKQQKSAFKTSLDDDPSQSANLLAEAKKLNDAQAPK

SEQ ID NO: 30 - protein, 58 aa, Artificial sequence
  Engineered CD69-binding polypeptide
VDNKFNKEFADAMAEILRLPNLNKYQKEAFKTSLKDDPSQSANLLAEAKKLNDAQAPK

SEQ ID NO: 31 - protein, 58 aa, Artificial sequence
  Engineered CD69-binding polypeptide
VDNKFNKEFEYAMREILKLPNLNKYQKEAFKTSLKDDPSQSANLLAEAKKLNDAQAPK

SEQ ID NO: 32 - protein, 58 aa, Artificial sequence
  Engineered CD69-binding polypeptide
VDNKFNKEFNKAMKEILSLPNLNKYQKEAFKTSLKDDPSQSANLLAEAKKLNDAQAPK

SEQ ID NO: 33 - protein, 58 aa, Artificial sequence
  Engineered CD69-binding polypeptide
VDNKFNKEFYHAMNEILSLPNLNKYQKEAFKTSLKDDPSQSANLLAEAKKLNDAQAPK

SEQ ID NO: 34 - protein, 58 aa, Artificial sequence
  Engineered CD69-binding polypeptide
VDNKFNKEFYHAMHEILRLPNLNQYQKEAFKTSLKDDPSQSANLLAEAKKLNDAQAPK

SEQ ID NO: 35 - protein, 58 aa, Artificial sequence
  Engineered CD69-binding polypeptide
VDNKFNKEFYHAMAEILKLPNLNQYQKEAFKTSLKDDPSQSANLLAEAKKLNDAQAPK

SEQ ID NO: 36 - protein, 58 aa, Artificial sequence
  Engineered CD69-binding polypeptide
VDNKFNKEWYHAMKEILKLPNLNQYQKEAFKTSLKDDPSQSANLLAEAKKLNDAQAPK

SEQ ID NO: 37 - protein, 58 aa, Artificial sequence
  Engineered CD69-binding polypeptide
VDNKFNKEFHHAMWEILKLPNLNSYQKEAFKTSLKDDPSQSANLLAEAKKLNDAQAPK

SEQ ID NO: 38 - protein, 58 aa, Artificial sequence
  Engineered CD69-binding polypeptide
VDNKFNKEFNHAMKEILKLPNLNAYQKEAFKTSLKDDPSQSANLLAEAKKLNDAQAPK

SEQ ID NO: 39 - protein, 58 aa, Artificial sequence
  Engineered CD69-binding polypeptide
VDNKFNKEFNHAMKEILKLPNLNSYQKEAFKTSLKDDPSQSANLLAEAKKLNDAQAPK

SEQ ID NO: 40 - protein, 58 aa, Artificial sequence
  Engineered CD69-binding polypeptide
VDNKFNKEFEHAMKEILKLPNLNKYQKEAFKTSLKDDPSQSANLLAEAKKLNDAQAPK

SEQ ID NO: 41 - protein, 58 aa, Artificial sequence
  Engineered CD69-binding polypeptide
VDNKFNKEFANAMKEILKLPNLNAYQKEAFKTSLKDDPSQSANLLAEAKKLNDAQAPK

SEQ ID NO: 42 - protein, 58 aa, Artificial sequence
  Engineered CD69-binding polypeptide
VDNKFNKEFHHAMKEILKLPNLNSYQKEAFKTSLKDDPSQSANLLAEAKKLNDAQAPK

SEQ ID NO: 43 - protein, 58 aa, Artificial sequence
  Engineered CD69-binding polypeptide
VDNKFNKEFTHAMKEILKLPNLNKYQKEAFKTSLKDDPSQSANLLAEAKKLNDAQAPK

SEQ ID NO: 44 - protein, 58 aa, Artificial sequence
  Engineered CD69-binding polypeptide
VDNKFNKEFNHAMKEILKLPNLNKYQKEAFKTSLKDDPSQSANLLAEAKKLNDAQAPK

SEQ ID NO: 45 - protein, 58 aa, Artificial sequence
  Engineered CD69-binding polypeptide
VDNKFNKEFYHAMKEILKLPNLNSYQKEAFKTSLKDDPSQSANLLAEAKKLNDAQAPK

SEQ ID NO: 46 - protein, 58 aa, Artificial sequence
  Engineered CD69-binding polypeptide
VDNKFNKEFADAMAEILRLPNLNKYQKEAFKTSLSDDPSQSANLLAEAKKLNDAQAPK

SEQ ID NO: 47 - protein, 58 aa, Artificial sequence
  Engineered CD69-binding polypeptide
VDNKFNKEFNHAMIEILSLPNLNVYQKEAFKTSLKDDPSQSANLLAEAKKLNDAQAPK

SEQ ID NO: 48 - protein, 58 aa, Artificial sequence
  Engineered CD69-binding polypeptide
VDNKFNKEFRHAMHEILSLPNLNKYQKEAFKTSLKDDPSQSANLLAEAKKLNDAQAPK

SEQ ID NO: 49 - protein, 58 aa, Artificial sequence
  Engineered CD69-binding polypeptide
VDNKFNKEFDLAMNEILKLPNLNQYQKEAFKTSLKDDPSQSANLLAEAKKLNDAQAPK

SEQ ID NO: 50 - protein, 58 aa, Artificial sequence
  Engineered CD69-binding polypeptide
VDNKFNKEWYDAMKEILKLPNLNKYQKEAFKTSLDDDPSQSANLLAEAKKLNDAQAPK

SEQ ID NO: 51 - protein, 58 aa, Artificial sequence
  Engineered CD69-binding polypeptide
VDNKFNKEFSHAMKEILRLPNLNTYQKEAFKTSLKDDPSQSANLLAEAKKLNDAQAPK

SEQ ID NO: 52 - protein, 58 aa, Artificial sequence
  Engineered CD69-binding polypeptide
VDNKFNKEFYHAMKEILKLPNLNKYQDEAFKHSLKDDPSQSANLLAEAKKLNDAQAPK

SEQ ID NO: 53 - protein, 58 aa, Artificial sequence
  Engineered CD69-binding polypeptide
VDNKFNKEFAHAMKEILKLPNLNKYQKEAFKFSLKDDPSQSANLLAEAKKLNDAQAPK

SEQ ID NO: 54 - protein, 58 aa, Artificial sequence
  Engineered CD69-binding polypeptide
VDNKFNKEFYHAMKEILRLPNLNMYQKEAFKTSLKDDPSQSANLLAEAKKLNDAQAPK

SEQ ID NO: 55 - protein, 58 aa, Artificial sequence
  Engineered CD69-binding polypeptide
VDNKFNKEFQSAMKEILTLPNLNKYQKEAFKTSLKDDPSQSANLLAEAKKLNDAQAPK

SEQ ID NO: 56 - protein, 58 aa, Artificial sequence
  Engineered CD69-binding polypeptide
VDNKFNKEFSHAMQEILTLPNLNKYQKEAFKTSLKDDPSQSANLLAEAKKLNDAQAPK

SEQ ID NO: 57 - protein, 58 aa, Artificial sequence
  Engineered CD69-binding polypeptide
VDNKFNKEFYHAMKEILKLPNLNQKQKEAFKTSLKDDPSQSANLLAEAKKLNDAQAPK

SEQ ID NO: 58 - protein, 58 aa, Artificial sequence
  Engineered CD69-binding polypeptide
VDNKFNKEFYHAMLEILKLPNLNQYQKEAFKTSLKDDPSQSANLLAEAKKLNDAQAPK

SEQ ID NO: 59 - protein, 58 aa, Artificial sequence
  Engineered CD69-binding polypeptide
VDNKFNKEMYHAWKEILVLPNLNKYQKEAFKTSLKDDPSQSANLLAEAKKLNDAQAPK

SEQ ID NO: 60 - protein, 58 aa, Artificial sequence
  Engineered CD69-binding polypeptide
VDNKFNKEFYHAMKEILKLPNLNPYQKEAFKTSLKDDPSQSANLLAEAKKLNDAQAPK

SEQ ID NO: 61 - protein, 58 aa, Artificial sequence
  Engineered CD69-binding polypeptide
VDNKFNKEFYTAMKEILKLPNLNKYQKEAFKTSLKDDPSQSANLLAEAKKLNDAQAPK

SEQ ID NO: 62 - protein, 58 aa, Artificial sequence
  Engineered CD69-binding polypeptide
VDNKFNKEFYVAMKEILKLPNLNKYQKEAFHTSLKDDPSQSANLLAEAKKLNDAQAPK

SEQ ID NO: 63 - protein, 58 aa, Artificial sequence
  Engineered CD69-binding polypeptide
VDNKFNKEFDRAWYEIAKLPNLNKYQKEAFKTSLKDDPSQSANLLAEAKKLNDAQAPK

SEQ ID NO: 64 - protein, 58 aa, Artificial sequence
  Engineered CD69-binding polypeptide
VDNKFNKEVVHAWKEILILPNLNKYQKEAFKTSLKDDPSQSANLLAEAKKLNDAQAPK

SEQ ID NO: 65 - protein, 58 aa, Artificial sequence
  Engineered CD69-binding polypeptide
VDNKFNKEFHHAMKEILLLPNLNKRQKEAFKTSLKDDPSQSANLLAEAKKLNDAQAPK

SEQ ID NO: 66 - protein, 58 aa, Artificial sequence
  Engineered CD69-binding polypeptide
VDNKFNKEFYHAWAEILKLPNLNRYQKFAFKTSLKDDPSQSANLLAEAKKLNDAQAPK

SEQ ID NO: 67 - protein, 58 aa, Artificial sequence
  Engineered CD69-binding polypeptide
VDNKFNKEFYHAMKEILVLPNLNHYQKEAFKTSLHDDPSQSANLLAEAKKLNDAQAPK

SEQ ID NO: 68 - protein, 58 aa, Artificial sequence
  Engineered CD69-binding polypeptide
VDNKFNKEFYHAMKEILKLPNLNQYQKEAFITSLRDDPSQSANLLAEAKKLNDAQAPK

SEQ ID NO: 69 - protein, 58 aa, Artificial sequence
  Engineered CD69-binding polypeptide
VDNKFNKEFYHAMKEILKLPNLNKYQKLAFKTSLLDDPSQSANLLAEAKKLNDAQAPK

SEQ ID NO: 70 - protein, 58 aa, Artificial sequence
  Engineered CD69-binding polypeptide
VDNKFNKEFYHAKKEILFLPNLNKYQKEAFKTSLKDDPSQSANLLAEAKKLNDAQAPK

SEQ ID NO: 71 - protein, 58 aa, Artificial sequence
  Engineered CD69-binding polypeptide
VDNKFNKEFYHAMKEILALPNLNKYQLEAFYTSLKDDPSQSANLLAEAKKLNDAQAPK

SEQ ID NO: 72 - protein, 58 aa, Artificial sequence
  Engineered CD69-binding polypeptide
VDNKFNKEFYHARREIIKLPNLNQYQKEAFKTSLKDDPSQSANLLAEAKKLNDAQAPK

SEQ ID NO: 73 - protein, 58 aa, Artificial sequence
  Engineered CD69-binding polypeptide
VDNKFNKEFYNAWWEIRKLPNLNAWQKEAFKTSLKDDPSQSSELLSEAKQLNDSQAPK

SEQ ID NO: 74 - protein, 58 aa, Artificial sequence
  Engineered CD69-binding polypeptide
VDNKFNKEFYNAWWEIRKLPNLNSYQKEAFKTSLKDDPSQSSELLSEAKQLNDSQAPK

SEQ ID NO: 75 - protein, 58 aa, Artificial sequence
  Engineered CD69-binding polypeptide
VDNKFNKEFQWAWQEILHLPNLNKYQKEAFKTSLKDDPSQSSELLSEAKQLNDSQAPK

SEQ ID NO: 76 - protein, 58 aa, Artificial sequence
  Engineered CD69-binding polypeptide
VDNKFNKEFSMAMKEILALPNLNQYQKEAFKTSLKDDPSQSSELLSEAKQLNDSQAPK

SEQ ID NO: 77 - protein, 58 aa, Artificial sequence
  Engineered CD69-binding polypeptide
VDNKFNKEFQWAWQEILVLPNLNKYQKEAFKTSLKDDPSQSSELLSEAKQLNDSQAPK

SEQ ID NO: 78 - protein, 58 aa, Artificial sequence
  Engineered CD69-binding polypeptide
VDNKFNKEFYHAMKEILKLPNLNKYQKEAFKTSLKDDPSQSSELLSEAKQLNDSQAPK

SEQ ID NO: 79 - protein, 58 aa, Artificial sequence
  Engineered CD69-binding polypeptide
VDNKFNKEFADAMAEILRLPNLNQYQKEAFKTSLKDDPSQSSELLSEAKQLNDSQAPK

SEQ ID NO: 80 - protein, 58 aa, Artificial sequence
  Engineered CD69-binding polypeptide
VDNKFNKEWYAAMNEILKLPNLNKYQKEAFKTSLKDDPSQSSELLSEAKQLNDSQAPK

SEQ ID NO: 81 - protein, 58 aa, Artificial sequence
  Engineered CD69-binding polypeptide
VDNKFNKEFYAAMNEILKLPNLNVYQKEAFKTSLKDDPSQSSELLSEAKQLNDSQAPK

SEQ ID NO: 82 - protein, 58 aa, Artificial sequence
  Engineered CD69-binding polypeptide
VDNKFNKEFHYAWHEILVLPNLNKYQKEAFKTSLKDDPSQSSELLSEAKQLNDSQAPK

SEQ ID NO: 83 - protein, 58 aa, Artificial sequence
  Engineered CD69-binding polypeptide
VDNKFNKEFYDAMNEILKLPNLNKYQKEAFKTSLKDDPSQSSELLSEAKQLNDSQAPK

SEQ ID NO: 84 - protein, 58 aa, Artificial sequence
  Engineered CD69-binding polypeptide
VDNKFNKEVYMAYQEILKLPNLNKYQKEAFKTSLKDDPSQSSELLSEAKQLNDSQAPK

SEQ ID NO: 85 - protein, 58 aa, Artificial sequence
  Engineered CD69-binding polypeptide
VDNKFNKEFHEAMKEILKLPNLNQYQKEAFKTSLKDDPSQSSELLSEAKQLNDSQAPK

SEQ ID NO: 86 - protein, 58 aa, Artificial sequence
  Engineered CD69-binding polypeptide
VDNKFNKEFEHAMKEILRLPNLNAYQKEAFKTSLKDDPSQSSELLSEAKQLNDSQAPK

SEQ ID NO: 87 - protein, 58 aa, Artificial sequence
  Engineered CD69-binding polypeptide
VDNKFNKEFTHAMYEILKLPNLNQYQKEAFKTSLKDDPSQSSELLSEAKQLNDSQAPK

SEQ ID NO: 88 - protein, 58 aa, Artificial sequence
  Engineered CD69-binding polypeptide
VDNKFNKEFDHAMREILKLPNLNQYQKEAFKTSLKDDPSQSSELLSEAKQLNDSQAPK

SEQ ID NO: 89 - protein, 58 aa, Artificial sequence
  Engineered CD69-binding polypeptide
VDNKFNKEFYDAMKEILKLPNLNKYQKEAFKTSLKDDPSQSSELLSEAKQLNDSQAPK

SEQ ID NO: 90 - protein, 58 aa, Artificial sequence
  Engineered CD69-binding polypeptide
VDNKFNKEFYHAMHEILKLPNLNKYQKEAFKTSLKDDPSQSSELLSEAKQLNDSQAPK

SEQ ID NO: 91 - protein, 58 aa, Artificial sequence
  Engineered CD69-binding polypeptide
VDNKFNKEFHHAMKEILKLPNLNKYQKEAFKTSLKDDPSQSSELLSEAKQLNDSQAPK

SEQ ID NO: 92 - protein, 58 aa, Artificial sequence
  Engineered CD69-binding polypeptide
VDNKFNKEFYVAMKEILKLPNLNQYQKEAFKTSLKDDPSQSSELLSEAKQLNDSQAPK

SEQ ID NO: 93 - protein, 58 aa, Artificial sequence
  Engineered CD69-binding polypeptide
VDNKFNKEFYHAMKEILSLPNLNQYQKEAFKTSLKDDPSQSSELLSEAKQLNDSQAPK

SEQ ID NO: 94 - protein, 58 aa, Artificial sequence
  Engineered CD69-binding polypeptide
VDNKFNKEFYEAMKEILKLPNLNQYQKEAFKTSLKDDPSQSSELLSEAKQLNDSQAPK

SEQ ID NO: 95 - protein, 58 aa, Artificial sequence
  Engineered CD69-binding polypeptide
VDNKFNKEFYSAMKEILHLPNLNSYQKEAFKTSLKDDPSQSSELLSEAKQLNDSQAPK

SEQ ID NO: 96 - protein, 58 aa, Artificial sequence
  Engineered CD69-binding polypeptide
VDNKFNKEFYHAMNEILKLPNLNQYQKEAFKTSLKDDPSQSSELLSEAKQLNDSQAPK

SEQ ID NO: 97 - protein, 58 aa, Artificial sequence
  Engineered CD69-binding polypeptide
VDNKFNKEFSHAMKEILKLPNLNQYQKEAFKTSLKDDPSQSSELLSEAKQLNDSQAPK

SEQ ID NO: 98 - protein, 58 aa, Artificial sequence
  Engineered CD69-binding polypeptide
VDNKFNKEFYHAMKEILKLPNLNQYQKEAFKTSLKDDPSQSSELLSEAKQLNDSQAPK

SEQ ID NO: 99 - protein, 58 aa, Artificial sequence
  Engineered CD69-binding polypeptide
VDNKFNKEHYKAMAEILKLPNLNDYQLEAFKHSLKDDPSQSSELLSEAKQLNDSQAPK

SEQ ID NO: 100 - protein, 58 aa, Artificial sequence
  Engineered CD69-binding polypeptide
VDNKFNKEWAVAMREILNLPNLNAYQKEAFKTSLKDDPSQSSELLSEAKQLNDSQAPK

SEQ ID NO: 101 - protein, 58 aa, Artificial sequence
  Engineered CD69-binding polypeptide
VDNKFNKEFYAAMKEILKLPNLNKQQKSAFKTSLDDDPSQSSELLSEAKQLNDSQAPK

SEQ ID NO: 102 - protein, 58 aa, Artificial sequence
  Engineered CD69-binding polypeptide
VDNKFNKEFADAMAEILRLPNLNKYQKEAFKTSLKDDPSQSSELLSEAKQLNDSQAPK

SEQ ID NO: 103 - protein, 58 aa, Artificial sequence
  Engineered CD69-binding polypeptide
VDNKFNKEFEYAMREILKLPNLNKYQKEAFKTSLKDDPSQSSELLSEAKQLNDSQAPK

SEQ ID NO: 104 - protein, 58 aa, Artificial sequence
  Engineered CD69-binding polypeptide
VDNKFNKEFNKAMKEILSLPNLNKYQKEAFKTSLKDDPSQSSELLSEAKQLNDSQAPK

SEQ ID NO: 105 - protein, 58 aa, Artificial sequence
  Engineered CD69-binding polypeptide
VDNKFNKEFYHAMNEILSLPNLNKYQKEAFKTSLKDDPSQSSELLSEAKQLNDSQAPK

SEQ ID NO: 106 - protein, 58 aa, Artificial sequence
  Engineered CD69-binding polypeptide
VDNKFNKEFYHAMHEILRLPNLNQYQKEAFKTSLKDDPSQSSELLSEAKQLNDSQAPK

SEQ ID NO: 107 - protein, 58 aa, Artificial sequence
  Engineered CD69-binding polypeptide
VDNKFNKEFYHAMAEILKLPNLNQYQKEAFKTSLKDDPSQSSELLSEAKQLNDSQAPK

SEQ ID NO: 108 - protein, 58 aa, Artificial sequence
  Engineered CD69-binding polypeptide
VDNKFNKEWYHAMKEILKLPNLNQYQKEAFKTSLKDDPSQSSELLSEAKQLNDSQAPK

SEQ ID NO: 109 - protein, 58 aa, Artificial sequence
  Engineered CD69-binding polypeptide
VDNKFNKEFHHAMWEILKLPNLNSYQKEAFKTSLKDDPSQSSELLSEAKQLNDSQAPK

SEQ ID NO: 110 - protein, 58 aa, Artificial sequence
  Engineered CD69-binding polypeptide
VDNKFNKEFNHAMKEILKLPNLNAYQKEAFKTSLKDDPSQSSELLSEAKQLNDSQAPK

SEQ ID NO: 111 - protein, 58 aa, Artificial sequence
  Engineered CD69-binding polypeptide
VDNKFNKEFNHAMKEILKLPNLNSYQKEAFKTSLKDDPSQSSELLSEAKQLNDSQAPK

SEQ ID NO: 112 - protein, 58 aa, Artificial sequence
  Engineered CD69-binding polypeptide
VDNKFNKEFEHAMKEILKLPNLNKYQKEAFKTSLKDDPSQSSELLSEAKQLNDSQAPK

SEQ ID NO: 113 - protein, 58 aa, Artificial sequence
  Engineered CD69-binding polypeptide
VDNKFNKEFANAMKEILKLPNLNAYQKEAFKTSLKDDPSQSSELLSEAKQLNDSQAPK

SEQ ID NO: 114 - protein, 58 aa, Artificial sequence
  Engineered CD69-binding polypeptide
VDNKFNKEFHHAMKEILKLPNLNSYQKEAFKTSLKDDPSQSSELLSEAKQLNDSQAPK

SEQ ID NO: 115 - protein, 58 aa, Artificial sequence
  Engineered CD69-binding polypeptide
VDNKFNKEFTHAMKEILKLPNLNKYQKEAFKTSLKDDPSQSSELLSEAKQLNDSQAPK

SEQ ID NO: 116 - protein, 58 aa, Artificial sequence
  Engineered CD69-binding polypeptide
VDNKFNKEFNHAMKEILKLPNLNKYQKEAFKTSLKDDPSQSSELLSEAKQLNDSQAPK

SEQ ID NO: 117 - protein, 58 aa, Artificial sequence
  Engineered CD69-binding polypeptide
VDNKFNKEFYHAMKEILKLPNLNSYQKEAFKTSLKDDPSQSSELLSEAKQLNDSQAPK

SEQ ID NO: 118 - protein, 58 aa, Artificial sequence
  Engineered CD69-binding polypeptide
VDNKFNKEFADAMAEILRLPNLNKYQKEAFKTSLSDDPSQSSELLSEAKQLNDSQAPK

SEQ ID NO: 119 - protein, 58 aa, Artificial sequence
  Engineered CD69-binding polypeptide
VDNKFNKEFNHAMIEILSLPNLNVYQKEAFKTSLKDDPSQSSELLSEAKQLNDSQAPK

SEQ ID NO: 120 - protein, 58 aa, Artificial sequence
  Engineered CD69-binding polypeptide
VDNKFNKEFRHAMHEILSLPNLNKYQKEAFKTSLKDDPSQSSELLSEAKQLNDSQAPK

SEQ ID NO: 121 - protein, 58 aa, Artificial sequence
  Engineered CD69-binding polypeptide
VDNKFNKEFDLAMNEILKLPNLNQYQKEAFKTSLKDDPSQSSELLSEAKQLNDSQAPK

SEQ ID NO: 122 - protein, 58 aa, Artificial sequence
  Engineered CD69-binding polypeptide
VDNKFNKEWYDAMKEILKLPNLNKYQKEAFKTSLDDDPSQSSELLSEAKQLNDSQAPK

SEQ ID NO: 123 - protein, 58 aa, Artificial sequence
  Engineered CD69-binding polypeptide
VDNKFNKEFSHAMKEILRLPNLNTYQKEAFKTSLKDDPSQSSELLSEAKQLNDSQAPK

SEQ ID NO: 124 - protein, 58 aa, Artificial sequence
  Engineered CD69-binding polypeptide
VDNKFNKEFYHAMKEILKLPNLNKYQDEAFKHSLKDDPSQSSELLSEAKQLNDSQAPK

SEQ ID NO: 125 - protein, 58 aa, Artificial sequence
  Engineered CD69-binding polypeptide
VDNKFNKEFAHAMKEILKLPNLNKYQKEAFKFSLKDDPSQSSELLSEAKQLNDSQAPK

SEQ ID NO: 126 - protein, 58 aa, Artificial sequence
  Engineered CD69-binding polypeptide
VDNKFNKEFYHAMKEILRLPNLNMYQKEAFKTSLKDDPSQSSELLSEAKQLNDSQAPK

SEQ ID NO: 127 - protein, 58 aa, Artificial sequence
  Engineered CD69-binding polypeptide
VDNKFNKEFQSAMKEILTLPNLNKYQKEAFKTSLKDDPSQSSELLSEAKQLNDSQAPK

SEQ ID NO: 128 - protein, 58 aa, Artificial sequence
  Engineered CD69-binding polypeptide
VDNKFNKEFSHAMQEILTLPNLNKYQKEAFKTSLKDDPSQSSELLSEAKQLNDSQAPK

SEQ ID NO: 129 - protein, 58 aa, Artificial sequence
  Engineered CD69-binding polypeptide
VDNKFNKEFYHAMKEILKLPNLNQKQKEAFKTSLKDDPSQSSELLSEAKQLNDSQAPK

SEQ ID NO: 130 - protein, 58 aa, Artificial sequence
  Engineered CD69-binding polypeptide
VDNKFNKEFYHAMLEILKLPNLNQYQKEAFKTSLKDDPSQSSELLSEAKQLNDSQAPK

SEQ ID NO: 131 - protein, 58 aa, Artificial sequence
  Engineered CD69-binding polypeptide
VDNKFNKEMYHAWKEILVLPNLNKYQKEAFKTSLKDDPSQSSELLSEAKQLNDSQAPK

SEQ ID NO: 132 - protein, 58 aa, Artificial sequence
  Engineered CD69-binding polypeptide
VDNKFNKEFYHAMKEILKLPNLNPYQKEAFKTSLKDDPSQSSELLSEAKQLNDSQAPK

SEQ ID NO: 133 - protein, 58 aa, Artificial sequence
  Engineered CD69-binding polypeptide
VDNKFNKEFYTAMKEILKLPNLNKYQKEAFKTSLKDDPSQSSELLSEAKQLNDSQAPK

SEQ ID NO: 134 - protein, 58 aa, Artificial sequence
  Engineered CD69-binding polypeptide
VDNKFNKEFYVAMKEILKLPNLNKYQKEAFHTSLKDDPSQSSELLSEAKQLNDSQAPK

SEQ ID NO: 135 - protein, 58 aa, Artificial sequence
  Engineered CD69-binding polypeptide
VDNKFNKEFDRAWYEIAKLPNLNKYQKEAFKTSLKDDPSQSSELLSEAKQLNDSQAPK

SEQ ID NO: 136 - protein, 58 aa, Artificial sequence
  Engineered CD69-binding polypeptide
VDNKFNKEVVHAWKEILILPNLNKYQKEAFKTSLKDDPSQSSELLSEAKQLNDSQAPK

SEQ ID NO: 137 - protein, 58 aa, Artificial sequence
  Engineered CD69-binding polypeptide
VDNKFNKEFHHAMKEILLLPNLNKRQKEAFKTSLKDDPSQSSELLSEAKQLNDSQAPK

SEQ ID NO: 138 - protein, 58 aa, Artificial sequence
  Engineered CD69-binding polypeptide
VDNKFNKEFYHAWAEILKLPNLNRYQKFAFKTSLKDDPSQSSELLSEAKQLNDSQAPK

SEQ ID NO: 139 - protein, 58 aa, Artificial sequence
  Engineered CD69-binding polypeptide
VDNKFNKEFYHAMKEILVLPNLNHYQKEAFKTSLHDDPSQSSELLSEAKQLNDSQAPK

SEQ ID NO: 140 - protein, 58 aa, Artificial sequence
  Engineered CD69-binding polypeptide
VDNKFNKEFYHAMKEILKLPNLNQYQKEAFITSLRDDPSQSSELLSEAKQLNDSQAPK

SEQ ID NO: 141 - protein, 58 aa, Artificial sequence
  Engineered CD69-binding polypeptide
VDNKFNKEFYHAMKEILKLPNLNKYQKLAFKTSLLDDPSQSSELLSEAKQLNDSQAPK

SEQ ID NO: 142 - protein, 58 aa, Artificial sequence
  Engineered CD69-binding polypeptide
VDNKFNKEFYHAKKEILFLPNLNKYQKEAFKTSLKDDPSQSSELLSEAKQLNDSQAPK

SEQ ID NO: 143 - protein, 58 aa, Artificial sequence
  Engineered CD69-binding polypeptide
VDNKFNKEFYHAMKEILALPNLNKYQLEAFYTSLKDDPSQSSELLSEAKQLNDSQAPK

SEQ ID NO: 144 - protein, 58 aa, Artificial sequence
  Engineered CD69-binding polypeptide
VDNKFNKEFYHARREIIKLPNLNQYQKEAFKTSLKDDPSQSSELLSEAKQLNDSQAPK

SEQ ID NO: 145 - protein, 74 aa, Artificial sequence
  Engineered CD69-binding polypeptide
MGSSHHHHHHYYLEVDNKFNKEFYHAMKEILKLPNLNKYQKEAFKTSLKDDPSQSANLLAEAKKLNDAQAPKVD

SEQ ID NO: 146 - protein, 75 aa, Artificial sequence
  Engineered CD69-binding polypeptide
MGSSHHHHHHYYLEVDNKFNKEFYHAMKEILKLPNLNKYQKEAFKTSLKDDPSQSANLLAEAKKLNDAQAPKVDC

SEQ ID NO: 147 - protein, 135 aa, Artificial sequence
  Engineered CD69-binding polypeptide
MGSSHHHHHHYYLEVDNKFNKEFYHAMKEILKLPNLNKYQKEAFKTSLKDDPSQSANLLAEAKKLNDAQAPKVDGGGGSGGGGSGGGGSLAEAKVLANRELDKYGVSDFYKRLINKAKTVEGVEALKLHILAALP

SEQ ID NO: 148 - protein, 74 aa, Artificial sequence
  Engineered CD69-binding polypeptide
MGSSHHHHHHYYLEVDNKFNKEFYNAWWEIRKLPNLNSYQKEAFKTSLKDDPSQSANLLAEAKKLNDAQAPKVD

SEQ ID NO: 149 - protein, 75 aa, Artificial sequence
  Engineered CD69-binding polypeptide
MGSSHHHHHHYYLEVDNKFNKEFYNAWWEIRKLPNLNSYQKEAFKTSLKDDPSQSANLLAEAKKLNDAQAPKVDC

SEQ ID NO: 150 - protein, 135 aa, Artificial sequence
  Engineered CD69-binding polypeptide
MGSSHHHHHHYYLEVDNKFNKEFYNAWWEIRKLPNLNSYQKEAFKTSLKDDPSQSANLLAEAKKLNDAQAPKVDGGGGSGGGGSGGGGSLAEAKVLANRELDKYGVSDFYKRLINKAKTVEGVEALKLHILAALP

SEQ ID NO: 151 - protein, 74 aa, Artificial sequence
  Engineered CD69-binding polypeptide
MGSSHHHHHHYYLEVDNKFNKEFQWAWQEILHLPNLNKYQKEAFKTSLKDDPSQSANLLAEAKKLNDAQAPKVD

SEQ ID NO: 152 - protein, 75 aa, Artificial sequence
  Engineered CD69-binding polypeptide
MGSSHHHHHHYYLEVDNKFNKEFQWAWQEILHLPNLNKYQKEAFKTSLKDDPSQSANLLAEAKKLNDAQAPKVDC

SEQ ID NO: 153 - protein, 135 aa, Artificial sequence
  Engineered CD69-binding polypeptide
MGSSHHHHHHYYLEVDNKFNKEFQWAWQEILHLPNLNKYQKEAFKTSLKDDPSQSANLLAEAKKLNDAQAPKVDGGGGSGGGGSGGGGSLAEAKVLANRELDKYGVSDFYKRLINKAKTVEGVEALKLHILAALP

SEQ ID NO: 154 - protein, 74 aa, Artificial sequence
  Engineered CD69-binding polypeptide
MGSSHHHHHHYYLEVDNKFNKEFSMAMKEILALPNLNQYQKEAFKTSLKDDPSQSANLLAEAKKLNDAQAPKVD

SEQ ID NO: 155 - protein, 75 aa, Artificial sequence
  Engineered CD69-binding polypeptide
MGSSHHHHHHYYLEVDNKFNKEFSMAMKEILALPNLNQYQKEAFKTSLKDDPSQSANLLAEAKKLNDAQAPKVDC

SEQ ID NO: 156 - protein, 135 aa, Artificial sequence
  Engineered CD69-binding polypeptide
MGSSHHHHHHYYLEVDNKFNKEFSMAMKEILALPNLNQYQKEAFKTSLKDDPSQSANLLAEAKKLNDAQAPKVDGGGGSGGGGSGGGGSLAEAKVLANRELDKYGVSDFYKRLINKAKTVEGVEALKLHILAALP

SEQ ID NO: 157 - protein, 74 aa, Artificial sequence
  Engineered CD69-binding polypeptide
MGSSHHHHHHYYLEVDNKFNKEFQWAWQEILVLPNLNKYQKEAFKTSLKDDPSQSANLLAEAKKLNDAQAPKVD

SEQ ID NO: 158 - protein, 75 aa, Artificial sequence
  Engineered CD69-binding polypeptide
MGSSHHHHHHYYLEVDNKFNKEFQWAWQEILVLPNLNKYQKEAFKTSLKDDPSQSANLLAEAKKLNDAQAPKVDC

SEQ ID NO: 159 - protein, 135 aa, Artificial sequence
  Engineered CD69-binding polypeptide
MGSSHHHHHHYYLEVDNKFNKEFQWAWQEILVLPNLNKYQKEAFKTSLKDDPSQSANLLAEAKKLNDAQAPKVDGGGGSGGGGSGGGGSLAEAKVLANRELDKYGVSDFYKRLINKAKTVEGVEALKLHILAALP

SEQ ID NO: 160 - protein, 74 aa, Artificial sequence; identical to SEQ ID NO: 145
  Engineered CD69-binding polypeptide

SEQ ID NO: 161 - protein, 75 aa, Artificial sequence; identical to SEQ ID NO: 146
  Engineered CD69-binding polypeptide

SEQ ID NO: 162 - protein, 135 aa, Artificial sequence; identical to SEQ ID NO: 147
  Engineered CD69-binding polypeptide

SEQ ID NO: 163 - protein, 58 aa, Artificial sequence
  Engineered Taq-binding polypeptide
VDNKFNKEKGEAVVEIFRLPNLNGRQVKAFIASLYDDPSQSANLLAEAKKLNDAQAPK

SEQ ID NO: 164 - protein, 46 aa, Artificial sequence
  Engineered albumin-binding polypeptide
LAEAKVLANRELDKYGVSDFYKRLINKAKTVEGVEALKLHILAALP

SEQ ID NO: 165 - protein, 145 aa, Homo sapiens
HHHHHHHHHGQYNCPGQYTFSMPSDSHVSSCSEDWVGYQRKCYFISTVKRSWTSAQNACSEHGATLAVIDSEKDMNFLKRYAGREEHWVGLKKEPGHPWKWSNGKEFNNWFNVTGSDKCVFLKNTEVSSMECEKNLYWICNKPYK

SEQ ID NO: 166 - protein, 145 aa, Mus musculus
HHHHHHHHHGKYNCPGLYEKLESSDHHVATCKNEWISYKRTCYFFSTTTKSWALAQRSCSEDAATLAVIDSEKDMTFLKRYSGELEHWIGLKNEANQTWKWANGKEFNSWFNLTGSGRCVSVNHKNVTAVDCEANFHWVCSKPSR

SEQ ID NO: 167 - protein, 609 aa, Homo sapiens [sequence omitted]

SEQ ID NO: 168 - protein, 29 aa, Artificial sequence
  Engineered CD69-binding polypeptide
  VARIANT (2)..(2): F, H, V or W
  VARIANT (3)..(3): A, D, E, H, N, Q, S, T or Y
  VARIANT (4)..(4): A, D, E, H, K, M, N, S, V, W or Y
  VARIANT (6)..(6): M, W or Y
  VARIANT (7)..(7): A, H, K, N, Q, R, W or Y
  VARIANT (10)..(10): L or R
  VARIANT (11)..(11): A, H, K, R, S or V
  VARIANT (16)..(16): N or T
  VARIANT (17)..(17): A, D, K, Q, S or V
  VARIANT (18)..(18): W or Y
  VARIANT (21)..(21): E or S
  VARIANT (25)..(25): H or T
  VARIANT (26)..(26): K or S
EXXXAXXEIXXLPNLXXXQKXAFKXXLKD

SEQ ID NO: 169 - protein, 16 aa, Artificial sequence
  Amino acid linker
GGGGSLVPRGSGGGGS

SEQ ID NO: 170 - protein, 6 aa, Artificial sequence
  Amino acid linker
GSGSGS

SEQ ID NO: 171 - protein, 8 aa, Artificial sequence
  Amino acid linker
GSGSGSGS

SEQ ID NO: 172 - protein, 16 aa, Artificial sequence
  Amino acid linker
GSGSGSGSGSGSGSGS

SEQ ID NO: 173 - protein, 11 aa, Artificial sequence
  Amino acid linker
GGSGGHMGSGG

SEQ ID NO: 174 - protein, 11 aa, Artificial sequence
  Amino acid linker
GGSGGSGGSGG

SEQ ID NO: 175 - protein, 5 aa, Artificial sequence
  Amino acid linker
GGSGG

SEQ ID NO: 176 - protein, 8 aa, Artificial sequence
  Amino acid linker
GGSGGGGG

SEQ ID NO: 177 - protein, 18 aa, Artificial sequence
  Amino acid linker
GGGSEGGGSEGGGSEGGG

SEQ ID NO: 178 - protein, 8 aa, Artificial sequence
  Amino acid linker
AAGAATAA

SEQ ID NO: 179 - protein, 5 aa, Artificial sequence
  Amino acid linker
GGGGG

SEQ ID NO: 180 - protein, 5 aa, Artificial sequence
  Amino acid linker
GGSSG

SEQ ID NO: 181 - protein, 11 aa, Artificial sequence
  Amino acid linker
GSGGGTGGGSG

SEQ ID NO: 182 - protein, 11 aa, Artificial sequence
  Amino acid linker
GSGGGTGGGSG

SEQ ID NO: 183 - protein, 12 aa, Artificial sequence
  Amino acid linker
GSGSGSGSGGSG

SEQ ID NO: 184 - protein, 14 aa, Artificial sequence
  Amino acid linker
GSGGSGGSGGSGGS

SEQ ID NO: 185 - protein, 14 aa, Artificial sequence
  Amino acid linker
GSGGSGSGGSGGSG

SEQ ID NO: 186 - protein, 5 aa, Artificial sequence
  Amino acid linker
SSSSG

SEQ ID NO: 187 - protein, 15 aa, Artificial sequence
  Amino acid linker
SSSSGSSSSGSSSSG

SEQ ID NO: 188 - protein, 20 aa, Artificial sequence
  Amino acid linker
SSSSGSSSSGSSSSGSSSSG

SEQ ID NO: 189 - protein, 5 aa, Artificial sequence
  Amino acid linker
GGGGS

SEQ ID NO: 190 - protein, 15 aa, Artificial sequence
  Amino acid linker
GGGGSGGGGSGGGGS